(19) United States
(12) Patent Application Publication
(10) Pub. No.: US 2017/0027897 A1
(43) Pub. Date: Feb. 2, 2017

(54) mTORC1 MODULATION BY AMINO ACIDS AND USES THEREOF

(71) Applicant: WHITEHEAD INSTITUTE FOR BIOMEDICAL RESEARCH, Cambridge, MA (US)

(72) Inventors: Shuyu Wang, Cambridge, MA (US); William Comb, Somerville, MA (US); Alejo Efeyan, Somerville, MA (US); Roberto Zoncu, Cambridge, MA (US); David M. Sabatini, Cambridge, MA (US)

(21) Appl. No.: 15/031,679

(22) PCT Filed: Oct. 23, 2014
(86) PCT No.: PCT/US14/62031

§ 371 Date: Apr. 22, 2016

Related U.S. Application Data
(60) Provisional application No. 61/894,744, filed on Oct. 23, 2013.

(51) Int. Cl. A61K 31/223; A61K 9/00; A23K 20/142; A23K 50/50; A23L 33/175; A61K 38/06; A23L 2/52

(52) U.S. Cl. CPC A61K 31/223; A61K 38/06; A61K 9/0053; A23L 2/52; A23K 50/50; A23L 33/175; A23K 20/142; A23V 2002/00

(57) ABSTRACT

Disclosed herein are methods for modulating mTORC1 activation in cells or a subject, and related compositions, kits, and agents for use in those methods. Also disclosed is the treatment of diseases and conditions characterized by decreased mTORC1 activation.

### RELATED APPLICATIONS

This application claims the benefit of U.S. Provisional Application No. 61/894,744, filed on Oct. 23, 2013. The entire teachings of the above application(s) are incorporated herein by reference

### GOVERNMENT SUPPORT

This invention was made with government support under R01 CA129105, R01 CA103866, and R01 AI047389 awarded by the National Institutes of Health. The government has certain rights in the invention.

### BACKGROUND OF THE INVENTION

The mammalian/mechanistic target of rapamycin (mTOR) is the serine/threonine kinase at the catalytic core of mTORC1, a multi-protein complex that promotes growth and metabolic homeostasis in response to amino acid availability, energy levels, growth factors, and cellular stress.¹ The relay of inputs like energy levels and growth factors to mTORC1 occurs via the Tsc/Rheb axis and has now been characterized in detail.²

mTORC1 is a multi-subunit kinase that possesses a large scaffolding protein known as regulatory associated protein of mTOR (Raptor), a small WD-40 repeat protein known as mLST8, as well as two regulatory proteins known as pRAs40 and DEPTOR, in addition to mTOR, which serves as the catalytic subunit for mTORC1. Mammalian target of rapamycin (mTOR) is a serine/threonine kinase that is evolutionally conserved and integrates multiple physiological pathways, such as nutrients (e.g., amino acids and glucose), growth factors (e.g., insulin like growth factor 1), hormones (e.g., leptin), and stresses, including for example starvation, hypoxia, and damage to DNA, in order to regulate various cell functions, namely translation, transcription, cellular growth, metabolism, survival, energy balance and response to stress. (Watanabe, et al. J. Nucl. Med. 52(4):497-500, 2011). mTOR pathway dysregulation is implicated with various disease (e.g., cancer). mTORC1 is responsible for phosphorylating S6K and 4E-BP1 (which may be used to assay for mTORC1 activity), and regulates numerous physiological processes, including, for example, translation, autophagy, growth, lipid biosynthesis, mitochondria biogenesis, and ribosome biogenesis. mTOR interacts with a specific component of mTORC1, known as Raptor, via binding to an N-terminal HEAT domain. Raptor serves as a scaffolding protein, and recruits SK6 and 4E-BP1 for the promotion of protein synthesis via direct phosphorylation by mTORC1 of S6K and 4E-BP1. A more detailed description of mTORC1, its mechanism of action, and various modulators of mTORC1 can be found in Liu et al. (Liu, et al. Drug Discov Today Ther Strateg. 6(2):47-55, 2009).

In recent years, our lab has demonstrated that amino acid signaling to mTORC1 requires the Rag GTPases and their associated regulatory factors.³⁻⁷ However, the molecular mechanism(s) by which amino acid sensing occurs remains mysterious and motivates the following work.

### SUMMARY OF THE INVENTION

In an aspect, the present invention provides a combination for increasing mTORC1 activity in a subject to increase muscle mass, increase muscle anabolism, or to treat a disease or condition selected from skeletal muscle atrophy, decreased satiety, abnormally high food intake, hyperphagia, ribosomopathies, cohesinopathies, and conditions that cause reduced myelination of nerves, the combination comprising: a first component selected from L-arginine; an mTORC1 agonizing arginine mimetic; and a peptide, non-standard peptide, polypeptide, non-standard polypeptide, protein or non-standard protein any of which is enriched for one or both of L-arginine residues or mTORC1 agonizing arginine mimetic residues; a second component selected from L-leucine; an mTORC1 agonizing leucine mimetic; and a peptide, non-standard peptide, polypeptide, non-standard polypeptide, protein or non-standard protein any of which is enriched for one or both of L-leucine residues or mTORC1 agonizing leucine mimetic residues; and optionally, a third component selected from L-lysine; an mTORC1 agonizing lysine mimetic; and a peptide, non-standard peptide, polypeptide, non-standard polypeptide, protein or non-standard protein any of which is enriched for one or both of L-lysine residues or mTORC1 agonizing lysine mimetic residues; wherein: each component is present in an acceptable form for administration to the subject; any two or all three components may be part of a single composition or a single molecule; and each component is co-administered with one another to the subject.

In some embodiments of the combination, the third component is administered to the subject. In some embodiments of the combination, at least one of the first component, second component or optional third component is other than a naturally occurring L-form of an amino acid. In some embodiments of the combination, the first component is L-arginine or an mTORC1 agonizing arginine mimetic selected from a carboxy terminal modified form of L-arginine and a side-chain modified form of L-arginine. In some embodiments of the combination, the first component is selected from: L-arginine, a L-arginine ester, citrulline

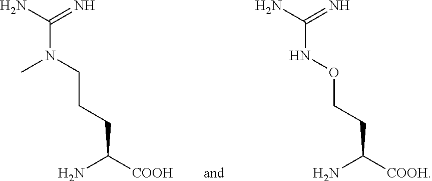

In some embodiments of the combination, the L-arginine ester is L-arginine ethyl ester. In some embodiments of the combination, the second component is L-leucine or an mTORC1 agonizing leucine mimetic selected from a carboxy terminal modified form of L-leucine, an amino terminal modified form of L-leucine, a side-chain modified form of L-leucine, and L-methionine. In some embodiments of the combination, the second component is selected from L-leucine, a L-leucine ester, L-methionine

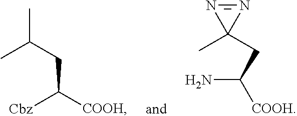

In some embodiments of the combination, the L-leucine ester is L-leucine ethyl ester. In some embodiments of the combination, the third component, if present, is selected from L-lysine and an L-lysine ester. In some embodiments of the combination, the L-lysine ester is L-lysine ethyl ester. In some embodiments of the combination, at least one component is selected from: a peptide, non-standard peptide, polypeptide, non-standard polypeptide, protein or non-standard protein any of which is enriched for one or both of L-arginine residues or mTORC1 agonizing arginine mimetic residues; a peptide, non-standard peptide, polypeptide, non-standard polypeptide, protein or non-standard protein any of which is enriched for one or both of L-leucine residues or mTORC1 agonizing leucine mimetic residues; and a peptide, non-standard peptide, polypeptide, non-standard polypeptide, protein or non-standard protein any of which is enriched for one or both of L-lysine residues or mTORC1 agonizing lysine mimetic residues; wherein: any peptide, non-standard peptide, polypeptide or non-standard polypeptide is optionally associated with one or more of a half-life increasing moiety, a cell penetration moiety, a specific organ-directing moiety, and a specific cell-type-directing moiety.

In some embodiments of the combination, the at least one component is selected from: a peptide, polypeptide, or protein any of which is enriched for L-arginine residues; a peptide, polypeptide, or protein any of which is enriched for L-leucine residues; and a peptide polypeptide, or protein any of which is enriched for L-lysine residues. In some embodiments of the combination, at least two components are independently selected from: a peptide, non-standard peptide, polypeptide, non-standard polypeptide, protein or non-standard protein any of which is enriched for one or both of L-arginine residues or mTORC1 agonizing arginine mimetic residues; a peptide, non-standard peptide, polypeptide, non-standard polypeptide, protein or non-standard protein any of which is enriched for one or both of L-leucine residues or mTORC1 agonizing leucine mimetic residues; and a peptide, non-standard peptide, polypeptide, non-standard polypeptide, protein or non-standard protein any of which is enriched for one or both of L-lysine residues or mTORC1 agonizing lysine mimetic residues.

In some embodiments of the combination, each of the at least two component is independently selected from: a peptide, polypeptide, or protein any of which is enriched for L-arginine residues; a peptide, polypeptide, or protein any of which is enriched for L-leucine residues; and a peptide polypeptide, or protein any of which is enriched for L-lysine residues.

In some embodiments of the combination, the third component is present and each of the first, second and third components are independently selected from: a peptide, non-standard peptide, polypeptide, non-standard polypeptide, protein or non-standard protein any of which is enriched for one or both of L-arginine residues or mTORC1 agonizing arginine mimetic residues; a peptide, non-standard peptide, polypeptide, non-standard polypeptide, protein or non-standard protein any of which is enriched for one or both of L-leucine residues or mTORC1 agonizing leucine mimetic residues; and a peptide, non-standard peptide, polypeptide, non-standard polypeptide, protein or non-standard protein any of which is enriched for one or both of L-lysine residues or mTORC1 agonizing lysine mimetic residues.

In some embodiments of the combination, each of the first, second and third components are independently selected from: a peptide, polypeptide, or protein any of which is enriched for L-arginine residues; a peptide, polypeptide, or protein any of which is enriched for L-leucine residues; and a peptide polypeptide, or protein any of which is enriched for L-lysine residues.

In some embodiments of the combination, the at least two components are present on one peptide, non-standard peptide, polypeptide, non-standard polypeptide, protein or non-standard protein which is enriched for at least two of: L-arginine residues or mTORC1 agonizing arginine mimetic residues; L-leucine residues or mTORC1 agonizing leucine mimetic residues; and L-lysine residues or mTORC1 agonizing lysine mimetic residues.

In some embodiments of the combination, the at least two components are present on one peptide, polypeptide, or protein which is enriched for at least two of: L-arginine residues; L-leucine residues; and L-lysine residues.

In some embodiments of the combination, each of the first, second and third components is present on one peptide, non-standard peptide, polypeptide, non-standard polypeptide, protein or non-standard protein which is enriched for L-arginine residues or mTORC1 agonizing arginine mimetic residues; L-leucine residues or mTORC1 agonizing leucine mimetic residues; and L-lysine residues or mTORC1 agonizing lysine mimetic residues. In some embodiments of the combination, each of the first, second and third components is present on one peptide, polypeptide, or protein which is enriched for L-arginine residues, L-leucine residues, and L-lysine residues. In some embodiments of the combination, the one peptide is a tripepetide consisting of the amino acid sequence Leu-Arg-Lys.

In some embodiments of the combination, every component is present on the same peptide, non-standard peptide, polypeptide or non-standard polypeptide, and wherein the peptide, non-standard peptide, polypeptide or non-standard polypeptide consists of residues selected from L-arginine residues, mTORC1 agonizing arginine mimetic residues, L-leucine residues or mTORC1 agonizing leucine mimetic residues, L-lysine residues and mTORC1 agonizing lysine mimetic residues; the peptide, non-standard peptide, polypeptide or non-standard polypeptide is optionally associated with one or more of a half-life increasing moiety, a cell penetration moiety, a specific organ-directing moiety, and a specific cell-type-directing moiety.

In some embodiments of the combination, the peptide, non-standard peptide, polypeptide, non-standard polypeptide, protein or non-standard protein comprises a cell penetration amino acid sequence. In some embodiments of the combination, every component is present on one peptide or polypeptide, and wherein the peptide or polypeptide consists of residues selected from L-arginine residues, L-leucine residues and, L-lysine residues; and, optionally, is associated with one or more of a half-life increasing moiety, a cell penetration moiety, a specific organ-directing moiety, and a specific cell-type-directing moiety. In some embodiments of the combination, every component is present on one peptide or polypeptide, and wherein the peptide or polypeptide comprises at least one mTORC1 agonizing arginine mimetic residue, mTORC1 agonizing leucine mimetic residue, or mTORC1 agonizing lysine mimetic residue; and, optionally is associated with one of more of a half-life increasing moiety, a cell penetration moiety, a specific organ-directing moiety, and a specific cell-type-directing moiety.

In some embodiments of the combination, the at least one mTORC1 agonizing arginine mimetic residue, mTORC1 agonizing leucine mimetic residue, or mTORC1 agonizing lysine mimetic residue is selected from a L-arginine ester, citrulline,

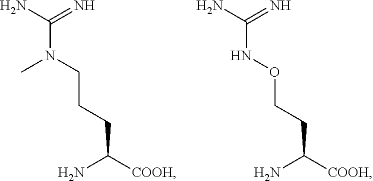

a L-arginine ester, L-methionine,

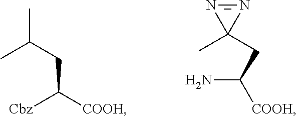

and corresponding monovalent and divalent radicals thereof.

In some embodiments of the combination, the peptide, non-standard peptide, polypeptide or non-standard polypeptide is between two and thirty residues in length. In some embodiments of the combination, the peptide, non-standard peptide, polypeptide or non-standard polypeptide is between two and twelve residues in length.

In some embodiments of the combination, each of the components is formulated into a pharmaceutically acceptable composition or a nutraceutical composition. In some embodiments of the combination, at least one of the components is formulated into a controlled release formulation. In some embodiments of the combination, at least one of the components is formulated into a composition to promote absorption from a specific portion of the digestive tract. In some embodiments of the combination, each component is formulated into either: a controlled release formulation; or a composition to promote absorption from a specific portion of the digestive tract. In some embodiments of the combination, at least one of the components is formulated into a pharmaceutical composition for delivery to a specific organ.

In some embodiments of the combination, the specific organ is the brain and each pharmaceutical composition is formulated to either cross the blood-brain barrier or for direct administration to the CNS. In some embodiments of the combination, the specific organ is muscle. In some embodiments of the combination, each of the components is formulated into a composition for oral administration. In some embodiments of the combination, each of the components is formulated into a composition for parenteral or intra-muscular administration. In some embodiments of the combination, each of the components is formulated into a composition that demonstrates an increase in Cₘₐₓ in the subject as compared to the Cₘₐₓ of a composition consisting of the corresponding component and a pharmaceutically acceptable buffer.

In some embodiments of the combination, prior to administration, information about the level of mTORC1 activity in the subject is received or obtained. In some embodiments of the combination, prior to administration, information about whether the subject is deficient in lysine is received or obtained. In some embodiments of the combination, the serum or cellular lysine levels of the subject is obtained and the choice of administering the third component is determined based on the obtained lysine levels. In some embodiments of the combination, each component is administered to the subject either a) prior to retiring for an extended sleep; or b) during sleeping hours. In some embodiments of the combination, each component is administered to the subject only during waking hours. In some embodiments of the combination, each component is administered to a fed subject. In some embodiments of the combination, each component is administered to a fasted subject.

In some embodiments, the combination is used to promote muscle anabolism, improve muscle function, increase muscle mass, reverse muscle atrophy or to prevent muscle atrophy. In some embodiments, the combination is used to reverse muscle atrophy or to prevent muscle atrophy due to inactivity, immobilization, or age of the subject or a disease or condition suffered by the subject. In some embodiments of the combination, the combination is used to reverse muscle atrophy or to prevent muscle atrophy due to a broken bone, a severe burn, a spinal injury, an amputation, a degenerative disease, a condition wherein recovery requires bed rest for the subject, a stay in an intensive care unit, or long-term hospitalization.

In some embodiments of the combination, the subject is suffering from a disease or condition known to be associated with cachexia and selected from cancer, AIDS, SARS, chronic heart failure, COPD, rheumatoid arthritis, liver disease, kidney disease and trauma. In some embodiments of the combination, the subject is suffering from a disease or condition known to be associated with malabsorption. In some embodiments of the combination, the disease or condition is selected from Crohn's disease, irritable bowel syndrome, celiac disease, and cystic fibrosis. In some embodiments of the combination, the subject is suffering from malnutrition, sarcopenia, muscle denervation, muscular dystrophy, an inflammatory myopathy, Spinal Muscle Atrophy, ALS, or myasthenia gravis. In some embodiments of the combination, the subject is preparing for, participating in or has recently returned from space travel. In some embodiments of the combination, the subject is preparing for, participating in or has recently returned from an armed conflict or military training.

In some embodiments, the combination is used to treat a ribosomopathy. In some embodiments of the combination, the ribosomopathy is selected from Diamond-Blackfan anemia, 5q-syndrome, Shwachman-Diamond syndrome, X-linked dyskeratosis, cartilage hair hypoplasia, and Treacher Collins syndrome.

In some embodiments, the combination is used to prevent autophagy in the patient. In some embodiments of the combination, the subject is suffering from cancer. In some embodiments, the combination is used to induce satiety in the subject. In some embodiments of the combination, the subject is suffering from obesity, diabetes or metabolic syndrome. In some embodiments of the combination, the subject is suffering from obesity. In some embodiments, the combination is used to increase strength and/or to increase muscle mass following exercise. In some embodiments, the combination is carried out in conjunction with physical therapy, as part of total parenteral nutrition, or to promote functional electrical stimulation.

In some embodiments of the combination, each of the components is present in a beverage or a nutrition bar.

In some embodiments of the combination, the subject is selected from a human and a companion animal. In some embodiments of the combination, the subject is selected from a human, a horse, a dog, or a cat. In some embodiments of the combination, the subject is a human. In some embodiments, the combination is used to increase the muscle-to-fat ratio in a non-human animal. In some embodiments, the combination is used to increase muscle mass in a non-human animal. In some embodiments of the combination, the non-human animal is selected from livestock or fish or poultry. In some embodiments of the combination, each of the components is administered as an additive to the feed of the non-human animal.

In an aspect, the present invention provides a method of increasing mTORC1 activity in a subject comprising administering to a subject in need thereof: a. a first component selected from L-arginine; an mTORC1 agonizing arginine mimetic; and a peptide, non-standard peptide, polypeptide, non-standard polypeptide, protein or non-standard protein any of which is enriched for one or both of L-arginine residues or mTORC1 agonizing arginine mimetic residues; b. a second component selected from L-leucine; an mTORC1 agonizing leucine mimetic; and a peptide, non-standard peptide, polypeptide, non-standard polypeptide, protein or non-standard protein any of which is enriched for one or both of L-leucine residues or mTORC1 agonizing leucine mimetic residues; and c. optionally, a third component selected from L-lysine; an mTORC1 agonizing lysine mimetic; and a peptide, non-standard peptide, polypeptide, non-standard polypeptide, protein or non-standard protein any of which is enriched for one or both of L-lysine residues or mTORC1 agonizing lysine mimetic residues; wherein: each component is present in an acceptable form for administration to the subject; any two or all three components may be part of a single composition or a single molecule; and each component is co-administered with one another to the subject.

In some embodiments, the third component is administered to the subject.

In some embodiments, at least one of the first component, second component or optional third component is other than a naturally occurring L-form of an amino acid. In some embodiments, the first component is L-arginine or an mTORC1 agonizing arginine mimetic selected from a carboxy terminal modified form of L-arginine and a side-chain modified form of L-arginine. In some embodiments, the first component is selected from: L-arginine, a L-arginine ester,

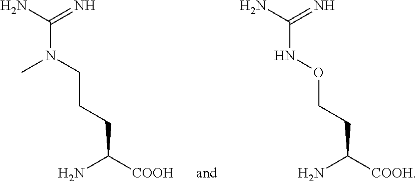

In some embodiments, the L-arginine ester is L-arginine ethyl ester.

In some embodiments, the second component is L-leucine or an mTORC1 agonizing leucine mimetic selected from a carboxy terminal modified form of L-leucine, an amino terminal modified form of L-leucine, a side-chain modified form of L-leucine, and L-methionine. In some embodiments, the second component is selected from L-leucine, a L-leucine ester, L-methionine,

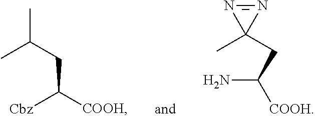

In some embodiments, the L-leucine ester is L-leucine ethyl ester.

In some embodiments, the third component, if present, is selected from L-lysine and an L-lysine ester. In some embodiments, the L-lysine ester is L-lysine ethyl ester. In some embodiments, at least one component is selected from: a. a peptide, non-standard peptide, polypeptide, non-standard polypeptide, protein or non-standard protein any of which is enriched for one or both of L-arginine residues or mTORC1 agonizing arginine mimetic residues; b. a peptide, non-standard peptide, polypeptide, non-standard polypeptide, protein or non-standard protein any of which is enriched for one or both of L-leucine residues or mTORC1 agonizing leucine mimetic residues; and c. a peptide, non-standard peptide, polypeptide, non-standard polypeptide, protein or non-standard protein any of which is enriched for one or both of L-lysine residues or mTORC1 agonizing lysine mimetic residues; wherein: any peptide, non-standard peptide, polypeptide or non-standard polypeptide is optionally associated with one or more of a half-life increasing moiety, a cell penetration moiety, a specific organ-directing moiety, and a specific cell-type-directing moiety. In some embodiments, the at least one component is selected from: a. a peptide, polypeptide, or protein any of which is enriched for L-arginine residues; b. a peptide, polypeptide, or protein any of which is enriched for L-leucine residues; and c. a peptide polypeptide, or protein any of which is enriched for L-lysine residues.

In some embodiments, at least two components are independently selected from: a. a peptide, non-standard peptide, polypeptide, non-standard polypeptide, protein or non-standard protein any of which is enriched for one or both of L-arginine residues or mTORC1 agonizing arginine mimetic residues; b. a peptide, non-standard peptide, polypeptide, non-standard polypeptide, protein or non-standard protein any of which is enriched for one or both of L-leucine residues or mTORC1 agonizing leucine mimetic residues; and c. a peptide, non-standard peptide, polypeptide, non-standard polypeptide, protein or non-standard protein any of which is enriched for one or both of L-lysine residues or mTORC1 agonizing lysine mimetic residues. In some embodiments, each of the at least two component is independently selected from: a. a peptide, polypeptide, or protein any of which is enriched for L-arginine residues; b. a peptide, polypeptide, or protein any of which is enriched for L-leucine residues; and c. a peptide polypeptide, or protein any of which is enriched for L-lysine residues.

In some embodiments, the third component is present and each of the first, second and third components are independently selected from: a. a peptide, non-standard peptide, polypeptide, non-standard polypeptide, protein or non-standard protein any of which is enriched for one or both of L-arginine residues or mTORC1 agonizing arginine mimetic residues; b. a peptide, non-standard peptide, polypeptide, non-standard polypeptide, protein or non-standard protein any of which is enriched for one or both of L-leucine residues or mTORC1 agonizing leucine mimetic residues; and c. a peptide, non-standard peptide, polypeptide, non-standard polypeptide, protein or non-standard protein any of which is enriched for one or both of L-lysine residues or mTORC1 agonizing lysine mimetic residues.

In some embodiments, each of the first, second and third components are independently selected from: a. a peptide, polypeptide, or protein any of which is enriched for L-arginine residues; b. a peptide, polypeptide, or protein any of which is enriched for L-leucine residues; and c. a peptide polypeptide, or protein any of which is enriched for L-lysine residues.

In some embodiments, the at least two components are present on one peptide, non-standard peptide, polypeptide, non-standard polypeptide, protein or non-standard protein which is enriched for at least two of: a. L-arginine residues or mTORC1 agonizing arginine mimetic residues; b. L-leucine residues or mTORC1 agonizing leucine mimetic residues; and c. L-lysine residues or mTORC1 agonizing lysine mimetic residues. In some embodiments, the at least two components are present on one peptide, polypeptide, or protein which is enriched for at least two of: a. L-arginine residues; b. L-leucine residues; and c. L-lysine residues.

In some embodiments, each of the first, second and third components is present on one peptide, non-standard peptide, polypeptide, non-standard polypeptide, protein or non-standard protein which is enriched for a. L-arginine residues or mTORC1 agonizing arginine mimetic residues; b. L-leucine residues or mTORC1 agonizing leucine mimetic residues; and c. L-lysine residues or mTORC1 agonizing lysine mimetic residues. In some embodiments, each of the first, second and third components is present on one peptide, polypeptide, or protein which is enriched for L-arginine residues, L-leucine residues, and L-lysine residues.

In some embodiments, every component is present on the same peptide, non-standard peptide, polypeptide or non-standard polypeptide, and wherein a. the peptide, non-standard peptide, polypeptide or non-standard polypeptide consists of residues selected from L-arginine residues, mTORC1 agonizing arginine mimetic residues, L-leucine residues or mTORC1 agonizing leucine mimetic residues, L-lysine residues and mTORC1 agonizing lysine mimetic residues; and b. the peptide, non-standard peptide, polypeptide or non-standard polypeptide is optionally associated with one or more of a half-life increasing moiety, a cell penetration moiety, a specific organ-directing moiety, and a specific cell-type-directing moiety. In some embodiments, the peptide, non-standard peptide, polypeptide, non-standard polypeptide, protein or non-standard protein comprises a cell penetration amino acid sequence. In some embodiments, every component is present on one peptide or polypeptide, and wherein the peptide or polypeptide consists of residues selected from L-arginine residues, L-leucine residues and, L-lysine residues; and, optionally, is associated with one or more of a half-life increasing moiety, a cell penetration moiety, a specific organ-directing moiety, and a specific cell-type-directing moiety. In some embodiments, every component is present on one peptide or polypeptide, and wherein the peptide or polypeptide comprises at least one mTORC1 agonizing arginine mimetic residue, mTORC1 agonizing leucine mimetic residue, or mTORC1 agonizing lysine mimetic residue; and, optionally is associated with one of more of a half-life increasing moiety, a cell penetration moiety, a specific organ-directing moiety, and a specific cell-type-directing moiety.

In some embodiments, the at least one mTORC1 agonizing arginine mimetic residue, mTORC1 agonizing leucine mimetic residue, or mTORC1 agonizing lysine mimetic residue is selected from a L-arginine ester

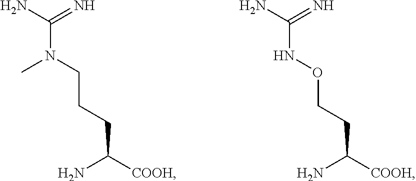

a L-leucine ester, L-methionine,

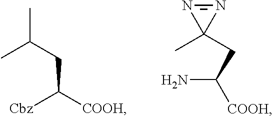

and corresponding monovalent and divalent radicals thereof.

In some embodiments, the peptide, non-standard peptide, polypeptide or non-standard polypeptide is between two and thirty residues in length. In some embodiments, the peptide, non-standard peptide, polypeptide or non-standard polypeptide is between two and twelve residues in length.

In some embodiments, each of the components is formulated into a pharmaceutically acceptable composition or a nutraceutical composition.

In some embodiments, at least one of the components is formulated into a controlled release formulation. In some embodiments, at least one of the components is formulated into a composition to promote absorption from a specific portion of the digestive tract. In some embodiments, each component is formulated into either: a. a controlled release formulation; or b. a composition to promote absorption from a specific portion of the digestive tract.

In some embodiments, at least one of the components is formulated into a pharmaceutical composition for delivery to a specific organ. In some embodiments, the specific organ is the brain and each pharmaceutical composition is formulated to either cross the blood-brain barrier or for direct administration to the CNS. In some embodiments, the specific organ is muscle.

In some embodiments, each of the components is formulated into a composition for oral administration. In some embodiments, each of the components is formulated into a composition for parenteral or intra-muscular administration. In some embodiments, each of the components is formulated into a composition that demonstrates an increase in Cₘₐₓ in the subject as compared to the Cₘₐₓ of a composition consisting of the corresponding component and a pharmaceutically acceptable buffer.

In some embodiments, prior to administration, information about the level of mTORC1 activity in the subject is received or obtained. In some embodiments, prior to administration, information about whether the subject is deficient in lysine is received or obtained. In some embodiments, the serum or cellular lysine levels of the subject is obtained and the choice of administering the third component is determined based on the obtained lysine levels.

In some embodiments, each component is administered to the subject either a) prior to retiring for an extended sleep; or b) during sleeping hours. In some embodiments, each component is administered to the subject only during waking hours. In some embodiments, each component is administered to a fed subject. In some embodiments, each component is administered to a fasted subject.

In some embodiments, the method is used to promote muscle anabolism, improve muscle function, increase muscle mass, reverse muscle atrophy or to prevent muscle atrophy. In some embodiments, the method is used to reverse muscle atrophy or to prevent muscle atrophy due to inactivity, immobilization, or age of the subject or a disease or condition suffered by the subject. In some embodiments, the method is used to reverse muscle atrophy or to prevent muscle atrophy due to a broken bone, a severe burn, a spinal injury, an amputation, a degenerative disease, a condition wherein recovery requires bed rest for the subject, a stay in an intensive care unit, or long-term hospitalization.

In some embodiments, the subject is suffering from a disease or condition known to be associated with cachexia and selected from cancer, AIDS, SARS, chronic heart failure, COPD, rheumatoid arthritis, liver disease, kidney disease and trauma. In some embodiments, the subject is suffering from a disease or condition known to be associated with malabsorption. In some embodiments, the disease or condition is selected from Crohn's disease, irritable bowel syndrome, celiac disease, and cystic fibrosis. In some embodiments, the subject is suffering from malnutrition, sarcopenia, muscle denervation, muscular dystrophy, an inflammatory myopathy, Spinal Muscle Atrophy, ALS, or myasthenia gravis. In some embodiments, the subject is preparing for, participating in or has recently returned from space travel. In some embodiments, the subject is preparing for, participating in or has recently returned from an armed conflict or military training.

In some embodiments, the method is used to treat a ribosomopathy. In some embodiments, the ribosomopathy is selected from Diamond-Blackfan anemia, 5q-syndrome, Shwachman-Diamond syndrome, X-linked dyskeratosis, cartilage hair hypoplasia, and Treacher Collins syndrome.

In some embodiments, the method is used to prevent autophagy in the patient. In some embodiments, the subject is suffering from cancer.

In some embodiments, the method is used to induce satiety (i.e., reduce food intake) in the subject. In some embodiments, the subject is suffering from a disease or condition characterized by reduced satiety (i.e, overeating, abnormally high food intake, hyperphagia), e.g., obesity, diabetes or metabolic syndrome. In some embodiments, the subject is suffering from obesity. In some embodiments, the subject is suffering from Down Syndrome and the method is used to reduce overeating associated with Down Syndrome. In some embodiments, the method is used to treat or prevent depression.

In some embodiments, each of the components is formulated for delivery to the brain of the subject.

In some embodiments, the method is used to treat or prevent jet lag.

In some embodiments, the method is used to prevent or reverse cardiac muscle atrophy in the subject. In some embodiments, the subject is suffering from or has suffered from a disease or condition selected from heart attack, congestive heart failure, heart transplant, heart valve repair, atherosclerosis, other major blood vessel disease, and heart bypass surgery.

In some embodiments, the method is used to increase strength and/or to increase muscle mass following exercise. In some embodiments, the method is carried out in conjunction with physical therapy, as part of total parenteral nutrition, or to promote functional electrical stimulation.

In some embodiments, each of the components is present in a beverage or a nutrition bar.

In some embodiments, the subject is selected from a human and a companion animal. In some embodiments, the subject is selected from a human, a horse, a dog, or a cat. In some embodiments, the subject is a human.

In some embodiments, the method is used to increase the muscle-to-fat ratio in a non-human animal. In some embodiments, the method is used to increase muscle mass in a non-human animal. In some embodiments, the non-human animal is selected from livestock or fish or poultry.

In some embodiments, each of the components is administered as an additive to the feed of the non-human animal.

In an aspect, the present invention provides a composition for administration to a subject consisting essentially of: a. a first component selected from L-arginine; an mTORC1 agonizing arginine mimetic; and a peptide, non-standard peptide, polypeptide, non-standard polypeptide, protein or non-standard protein any of which is enriched for one or both of L-arginine residues or mTORC1 agonizing arginine mimetic residues; and b. a second component selected from L-leucine; an mTORC1 agonizing leucine mimetic; and a peptide, non-standard peptide, polypeptide, non-standard polypeptide, protein or non-standard protein any of which is enriched for one or both of L-leucine residues or mTORC1 agonizing leucine mimetic residues; and c. optionally, a third component selected from L-lysine; an mTORC1 agonizing lysine mimetic; and a peptide, non-standard peptide, polypeptide, non-standard polypeptide, protein or non-standard protein any of which is enriched for one or both of L-lysine residues or mTORC1 agonizing lysine mimetic residues; and d. optionally, one or more excipients.

In some embodiments, the third component is present.

In some embodiments, at least one of the first component, second component or optional third component is other than a naturally occurring L-form of an amino acid.

In some embodiments, the first component is L-arginine or an mTORC1 agonizing arginine mimetic selected from a carboxy terminal modified form of L-arginine and a side-chain modified form of L-arginine. In some embodiments, the first component is selected from: L-arginine, a L-arginine ester,

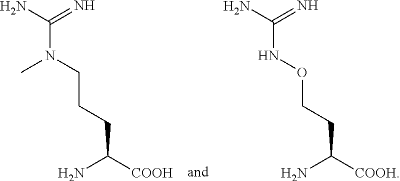

In some embodiments, the L-arginine ester is L-arginine ethyl ester.

In some embodiments, the second component is L-leucine or an mTORC1 agonizing leucine mimetic selected from a carboxy terminal modified form of L-leucine, an amino terminal modified form of L-leucine, a side-chain modified form of L-leucine, and L-methionine. In some embodiments, the second component is selected from L-leucine, a L-leucine ester, L-methionine,

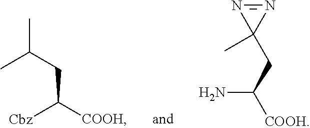

In some embodiments, the L-leucine ester is L-leucine ethyl ester.
In some embodiments, the third component is selected from L-lysine and an L-lysine ester. In some embodiments, the L-lysine ester is L-lysine ethyl ester.

In some embodiments, at least one component is selected from: a. a peptide, non-standard peptide, polypeptide, non-standard polypeptide, protein or non-standard protein any of which is enriched for one or both of L-arginine residues or mTORC1 agonizing arginine mimetic residues; b. a peptide, non-standard peptide, polypeptide, non-standard polypeptide, protein or non-standard protein any of which is enriched for one or both of L-leucine residues or mTORC1 agonizing leucine mimetic residues; and c. a peptide, non-standard peptide, polypeptide, non-standard polypeptide, protein or non-standard protein any of which is enriched for one or both of L-lysine residues or mTORC1 agonizing lysine mimetic residues; wherein: any peptide, non-standard peptide, polypeptide or non-standard polypeptide is optionally associated with one or more of a half-life increasing moiety, a cell penetration moiety, a specific organ-directing moiety, and a specific cell-type-directing moiety.

In some embodiments, at least one component is selected from a. a peptide, polypeptide, or protein any of which is enriched for L-arginine residues; b. a peptide, polypeptide, or protein any of which is enriched for L-leucine residues; and c. a peptide polypeptide, or protein any of which is enriched for L-lysine residues.

In some embodiments, at least two components are independently selected from: a. a peptide, non-standard peptide, polypeptide, non-standard polypeptide, protein or non-standard protein any of which is enriched for one or both of L-arginine residues or mTORC1 agonizing arginine mimetic residues; b. a peptide, non-standard peptide, polypeptide, non-standard polypeptide, protein or non-standard protein any of which is enriched for one or both of L-leucine residues or mTORC1 agonizing leucine mimetic residues; and c. a peptide, non-standard peptide, polypeptide, non-standard polypeptide, protein or non-standard protein any of which is enriched for one or both of L-lysine residues or mTORC1 agonizing lysine mimetic residues. In some embodiments, each of the at least two component is independently selected from: a. a peptide, polypeptide, or protein any of which is enriched for L-arginine residues; b. a peptide, polypeptide, or protein any of which is enriched for L-leucine residues; and c. a peptide polypeptide, or protein any of which is enriched for L-lysine residues.

In some embodiments, the third component is present and each of the first, second and third components are independently selected from: a. a peptide, non-standard peptide, polypeptide, non-standard polypeptide, protein or non-standard protein any of which is enriched for one or both of L-arginine residues or mTORC1 agonizing arginine mimetic residues; b. a peptide, non-standard peptide, polypeptide, non-standard polypeptide, protein or non-standard protein any of which is enriched for one or both of L-leucine residues or mTORC1 agonizing leucine mimetic residues; and c. a peptide, non-standard peptide, polypeptide, non-standard polypeptide, protein or non-standard protein any of which is enriched for one or both of L-lysine residues or mTORC1 agonizing lysine mimetic residues.

In some embodiments, each of the first, second and third components are independently selected from: a. a peptide, polypeptide, or protein any of which is enriched for L-arginine residues; b. a peptide, polypeptide, or protein any of which is enriched for L-leucine residues; and c. a peptide polypeptide, or protein any of which is enriched for L-lysine residues.

In some embodiments, the at least two components are present on one peptide, non-standard peptide, polypeptide, non-standard polypeptide, protein or non-standard protein which is enriched for at least two of: a. L-arginine residues or mTORC1 agonizing arginine mimetic residues; b. L-leucine residues or mTORC1 agonizing leucine mimetic residues; and c. L-lysine residues or mTORC1 agonizing lysine mimetic residues. In some embodiments, the at least two components are present on one peptide, polypeptide, or protein which is enriched for at least two of: a. L-arginine residues; b. L-leucine residues; and c. L-lysine residues.

In some embodiments, each of the first, second and third components is present on one peptide, non-standard peptide, polypeptide, non-standard polypeptide, protein or non-standard protein which is enriched for: a. L-arginine residues or mTORC1 agonizing arginine mimetic residues; b. L-leucine residues or mTORC1 agonizing leucine mimetic residues; and c. L-lysine residues or mTORC1 agonizing lysine mimetic residues. In some embodiments, each of the first, second and third components is present on one peptide, polypeptide, or protein which is enriched for L-arginine residues; L-leucine residues; and L-lysine residues.

In some embodiments, every component is present on the same peptide, non-standard peptide, polypeptide or non-standard polypeptide, wherein: a. the peptide, non-standard peptide, polypeptide or non-standard polypeptide, consists of residues selected from: L-arginine residues, mTORC1 agonizing arginine mimetic residues, L-leucine residues or mTORC1 agonizing leucine mimetic residues, L-lysine residues and mTORC1 agonizing lysine mimetic residues; b. the peptide, non-standard peptide, polypeptide or non-standard polypeptide, is optionally associated with one or more of a half-life increasing moiety, a cell penetration moiety, a specific organ-directing moiety, and a specific cell-type-directing moiety.

In some embodiments, the peptide, non-standard peptide, polypeptide, non-standard polypeptide, protein or non-standard protein further consists of a cell penetration amino acid sequence.

In some embodiments, every component is present on one peptide or polypeptide, and wherein the peptide or polypeptide consists of residues selected from L-arginine residues, L-leucine residues and, L-lysine residues; and, optionally is additionally associated with one or more of a half-life increasing moiety, a cell penetration moiety, a specific organ-directing moiety, and a specific cell-type-directing moiety. In some embodiments, every component is present on one peptide or polypeptide, and wherein the peptide or polypeptide comprises at least one mTORC1 agonizing arginine mimetic residue, mTORC1 agonizing leucine mimetic residue, or mTORC1 agonizing lysine mimetic residue; and, optionally is additionally associated with one or more of a half-life increasing moiety, a cell penetration moiety, a specific organ-directing moiety, and a specific cell-type-directing moiety.

In some embodiments, the at least one mTORC1 agonizing arginine mimetic residue, mTORC1 agonizing leucine mimetic residue, or mTORC1 agonizing lysine mimetic residue is selected from a L-arginine ester,

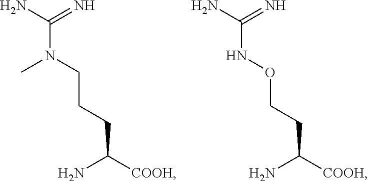

a L-leucine ester, L-methionine

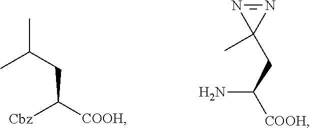

a L-lysine ester, and corresponding monovalent and divalent forms thereof.

In some embodiments, the peptide, non-standard peptide, polypeptide or non-standard polypeptide is between two and thirty residues in length. In some embodiments, the peptide, non-standard peptide, polypeptide or non-standard polypeptide is between two and twelve residues in length. In some embodiments, the peptide, non-standard peptide, polypeptide or non-standard polypeptide is the tripeptide Leu-Arg-Lys. In some embodiments, the peptide, non-standard peptide, polypeptide or non-standard polypeptide is the tripeptide Leu-Citrulline-Lys.

In some embodiments, the composition is a pharmaceutically acceptable composition or a nutraceutical composition.

In some embodiments, at least one of the components of the composition is formulated into a controlled release formulation. In some embodiments, at least one of the components of the composition is formulated to promote absorption from a specific portion of the digestive tract. In some embodiments, each component in the composition is formulated into either: a. a controlled release formulation; or b. an enterically coated composition to promote absorption from a specific portion of the digestive tract. In some embodiments, at least one of the components in the composition is formulated for delivery to a specific organ. In some embodiments, the specific organ is the brain; and the composition is formulated to either cross the blood-brain barrier or for direct administration to the CNS. In some embodiments, the organ is muscle. In some embodiments, the composition is formulated for oral administration. In some embodiments, the composition is formulated for parenteral or intra-muscular administration. In some embodiments, the composition is formulated to increase in Cₘₐₓ in the subject as compared to the Cₘₐₓ of a composition consisting of the corresponding components and a pharmaceutically acceptable buffer. In some embodiments, the composition is formulated as a pharmaceutically acceptable composition comprising one or more pharmaceutically acceptable excipients. In some embodiments, the composition is formulated as a food additive to be added to the feed of a non-human animal.

In some embodiments, the non-human animal is selected from a companion animal, livestock or fish.

In an aspect, the present invention provides a method of modulating mTORC1 activation in a cell, comprising modulating the levels of leucine, arginine, and lysine in the cell, wherein increasing the levels of leucine, arginine, and lysine in the cell stimulates activation of mTORC1 in the cell, and wherein decreasing the levels of leucine, arginine, and lysine in the cell suppresses activation of mTORC1 in the cell.

In an aspect, the present invention provides a method of stimulating mTORC1 activation in a cell, comprising contacting the cell with leucine, arginine, and lysine or a composition comprising leucine, arginine, and lysine.

In an aspect, the present invention provides a method of completing activation of mTORC1 in a cell, comprising contacting the cell with a combination of agents selected from the group consisting of: leucine, a metabolite of leucine, or a source of leucine; arginine, a metabolite of arginine, or a source of arginine; and lysine, a metabolite of lysine, or a source of lysine, or a composition comprising a combination of agents selected from the group consisting of: leucine, a metabolite of leucine, or a source of leucine; arginine, a metabolite of arginine, or a source of arginine; and lysine, a metabolite of lysine, or a source of lysine. In some embodiments, at least one of the leucine, arginine, and lysine, or source thereof is not a native form of leucine, arginine, or lysine. In more specific embodiments, the source of arginine is citrulline.

In an aspect, the present invention provides a method of modulating mTORC1 activation in a cell, comprising modulating the levels of a leucine mimetic, an arginine mimetic, and a lysine mimetic in a cell.

In some embodiments, modulating mTORC1 activation in the cell comprises stimulating mTORC1 activation in the cell. In some embodiments, stimulating mTORC1 activation in the cell comprises increasing the levels of the leucine mimetic, the arginine mimetic, and the lysine mimetic in the cell. In some embodiments, increasing the levels of the leucine mimetic, the arginine mimetic, and the lysine mimetic in the cell comprise contacting the cell with the leucine mimetic, the arginine mimetic, and the lysine mimetic.

In some embodiments, the leucine mimetic, the arginine mimetic, and the lysine mimetic each comprise, respectively, non-native forms of the amino acids leucine, arginine, and lysine. In some embodiments, at least one of the leucine mimetic, the arginine mimetic, and the lysine mimetic comprises a non-native amino form of the amino acids leucine, arginine, and lysine.

In some embodiments, the leucine mimetic, the arginine mimetic, and the lysine mimetic each comprise, respectively, native amino acids leucine, arginine, and lysine. In some embodiments, at least one of the leucine mimetic, the arginine mimetic, and the lysine mimetic comprises a native form of the respective amino acid.

In some embodiments, the leucine mimetic, the arginine mimetic, and the lysine mimetic each comprise, respectively, a peptide, polypeptide, or protein comprising native amino acids lysine, arginine, or lysine. In some embodiments, the peptide, polypeptide, or protein is not a protein selected from the group consisting of whey protein or whey protein isolate, soy protein or soy protein isolate, or casein or a caseinate. In some embodiments, the peptide, polypeptide, or protein is not an intact protein ingested, chewed, or digested during the course of a meal.

In some embodiments, the leucine mimetic, the arginine mimetic, and the lysine mimetic each comprise, respectively, a peptide, polypeptide, or protein enriched for native amino acids lysine, arginine, or lysine. In some embodiments, the peptide, polypeptide, or protein is not a protein selected from the group consisting of whey protein or whey protein isolate, soy protein or soy protein isolate, or casein or a caseinate. In some embodiments, the peptide, polypeptide, or protein is not an intact protein ingested, chewed, or digested during the course of a meal.

In some embodiments, the leucine mimetic, arginine mimetic, and lysine mimetic comprise a peptide, polypeptide, or protein comprising leucine, arginine, or lysine. In some embodiments, the peptide, polypeptide, or protein is not a protein selected from the group consisting of whey protein or whey protein isolate, soy protein or soy protein isolate, or casein or a caseinate. In some embodiments, the peptide, polypeptide, or protein is not an intact protein ingested, chewed, or digested during the course of a meal.

In some embodiments, the leucine mimetic, arginine mimetic, and lysine mimetic comprise a peptide, polypeptide, or protein enriched for leucine, arginine, or lysine. In some embodiments, the peptide, polypeptide, or protein is not a protein selected from the group consisting of whey protein or whey protein isolate, soy protein or soy protein isolate, or casein or a caseinate. In some embodiments, the peptide, polypeptide, or protein is not an intact protein ingested, chewed, or digested during the course of a meal.

In some embodiments, the leucine mimetic, the arginine mimetic, and the lysine mimetic each comprise, respectively, derivatives of leucine, arginine, or lysine. In some embodiments, the derivatives of leucine, arginine, and lysine each comprise, respectively, C-terminus modifications to leucine, arginine, or lysine. In some embodiments, the derivatives of leucine, arginine, and lysine each comprise, respectively, carboxy alkyls of leucine, arginine, or lysine. In some embodiments, the derivatives of leucine, arginine, and lysine each comprise, respectively, carboxy esters of leucine, arginine, or lysine. In some embodiments, the derivatives of leucine, arginine, and lysine each comprise, respectively, carboxy methyl esters of leucine, arginine, or lysine. In some embodiments, the derivatives of leucine, arginine, and lysine each comprise, respectively, carboxy ethyl esters of leucine, arginine, or lysine. In some embodiments, the derivatives of leucine, arginine, and lysine each comprise, respectively, N-terminus modifications to leucine, arginine, or lysine. In some embodiments, the derivatives of leucine, arginine, and lysine each comprise, respectively, leucine, arginine, or lysine modified by an amino bulky substituent group. In some embodiments, the derivatives of leucine, arginine, and lysine each comprise, respectively, leucine, arginine, or lysine modified by an amino carboxybenzyl (Cbz) protecting group. In some embodiments, the derivatives of leucine, arginine, and lysine each comprise, respectively, side-chain modifications to leucine, arginine, or lysine. In some embodiments, the leucine, arginine, and lysine derivatives each comprise, respectively, a photo-crosslinkable leucine, arginine, or lysine with a diazirine-modified side chain.

In some embodiments, the cell is a mammalian cell. In some embodiments, the cell is a human cell. In some embodiments, the cell is a HEK293T cell.

In some embodiments, the contacting occurs in vitro or ex vivo. In some embodiments, the contacting occurs in vivo.

In some embodiments, the composition comprises a pharmaceutically acceptable carrier, excipient or diluent. In some embodiments, the composition is administered orally, enterally, or parenterally. In some embodiments, the composition is selected from the group consisting of a food composition, a dietary supplement, a nutritional composition, a nutraceutical, a powdered nutritional product to be reconstituted in water or milk before consumption, a food additive, a medicament, a drink, and a pet food. In some embodiments, the food composition does not include a dietary source of leucine, arginine, or lysine, milk, milk protein, whey protein, whey protein isolate, soy protein, soy protein isolate, casein, or a caseinate.

In some embodiments, at least one of the leucine, arginine, and/or lysine comprises a non-native leucine, arginine, or lysine. In some embodiments, at least one of the leucine, arginine, and/or lysine in the composition is isolated and/or purified. In some embodiments, the composition comprises an additional active agent. In some embodiments, the additional active agent exerts an effect selected from the group consisting of increasing skeletal muscle mass, promoting skeletal muscle recovery, promoting skeletal muscle anabolism, treating or preventing muscle atrophy, and combinations thereof.

In an aspect, the present invention provides a method of increasing skeletal muscle mass, comprising contacting skeletal muscle with a leucine carboxy alkyl and arginine carboxy alkyl, or a composition comprising the leucine carboxy alkyl and the arginine carboxy alkyl.

In an aspect, the present invention provides a method of increasing skeletal muscle mass, comprising contacting skeletal muscle with a leucine alkyl ester and arginine alkyl ester, or a composition comprising the leucine alkyl ester and the arginine alkyl ester.

In an aspect, the present invention provides a method of increasing skeletal muscle mass, comprising contacting skeletal muscle with leucine ethyl ester and arginine ethyl ester, or a composition comprising leucine ethyl ester and arginine ethyl ester.

In an aspect, the present invention provides a method of increasing skeletal muscle mass in a subject, comprising administering to the subject an effective amount of a leucine carboxy alkyl and an arginine carboxy alkyl, or a composition comprising the leucine carboxy alkyl and the arginine carboxy alkyl.

In an aspect, the present invention provides a method of increasing skeletal muscle mass in a subject, comprising administering to the subject an effective amount of a leucine alkyl ester and arginine alkyl ester, or a composition comprising the leucine alkyl ester and the arginine alkyl ester.

In an aspect, the present invention provides a method of increasing skeletal muscle mass in a subject, comprising administering to the subject an effective amount of leucine ethyl ester and arginine ethyl ester, or an effective amount of a composition comprising leucine ethyl ester and arginine ethyl ester.

In an aspect, the present invention provides a method of increasing skeletal muscle mass in a subject, comprising administering to the subject an effective amount of leucine ethyl ester and arginine ethyl ester, or an effective amount of a composition comprising leucine ethyl ester and arginine ethyl ester.

In an aspect, the present invention provides a method of promoting skeletal muscle anabolism in a subject, comprising administering to the subject an effective amount of leucine carboxy alkyl and arginine carboxy alkyl, or an effective amount of a composition comprising leucine carboxy alkyl and arginine carboxy alkyl.

In an aspect, the present invention provides a method of promoting skeletal muscle anabolism in a subject, comprising administering to the subject an effective amount of leucine alkyl ester and arginine alkyl ester, or an effective amount of a composition comprising leucine alkyl ester and arginine alkyl ester.

In an aspect, the present invention provides a method of promoting skeletal muscle anabolism in a subject, comprising administering to the subject an effective amount of leucine ethyl ester and arginine ethyl ester, or an effective amount of a composition comprising leucine ethyl ester and arginine ethyl ester.

In an aspect, the present invention provides a method of promoting skeletal muscle recovery after immobilization-induced muscle atrophy in a subject, comprising administering to the subject an effective amount of leucine carboxy alkyl and arginine carboxy alkyl, or an effective amount of a composition comprising leucine carboxy alkyl and arginine carboxy alkyl.

In an aspect, the present invention provides a method of promoting skeletal muscle recovery after immobilization-induced muscle atrophy in a subject, comprising administering to the subject an effective amount of leucine alkyl ester and arginine alkyl ester, or an effective amount of a composition comprising leucine alkyl ester and arginine alkyl ester.

In an aspect, the present invention provides a method of promoting skeletal muscle recovery after immobilization-induced muscle atrophy in a subject, comprising administering to the subject an effective amount of leucine ethyl ester and arginine ethyl ester, or an effective amount of a composition comprising leucine ethyl ester and arginine ethyl ester.

In an aspect, the present invention provides a method of treating or preventing muscle atrophy in a subject, comprising administering to the subject an effective amount of leucine carboxy alkyl and arginine carboxy alkyl, or an effective amount of a composition comprising leucine carboxy alkyl and arginine carboxy alkyl.

In an aspect, the present invention provides a method of treating or preventing muscle atrophy in a subject, comprising administering to the subject an effective amount of leucine alkyl ester and arginine alkyl ester, or an effective amount of a composition comprising leucine alkyl ester and arginine alkyl ester.

In an aspect, the present invention provides a method of treating or preventing muscle atrophy in a subject, comprising administering to the subject an effective amount of leucine ethyl ester and arginine ethyl ester, or an effective amount of a composition comprising leucine ethyl ester and arginine ethyl ester.

In an aspect, the present invention provides a method of treating or preventing a disorder, condition, or symptom characterized by muscle atrophy in a subject, comprising administering to the subject an effective amount of leucine carboxy alkyl and arginine carboxy alkyl, or an effective amount of a composition comprising leucine carboxy alkyl and arginine carboxy alkyl.

In an aspect, the present invention provides a method of treating or preventing a disorder, condition, or symptom characterized by muscle atrophy in a subject, comprising administering to the subject an effective amount of leucine alkyl ester and arginine alkyl ester, or an effective amount of a composition comprising leucine alkyl ester and arginine alkyl ester.

In an aspect, the present invention provides a method of treating or preventing a disorder, condition, or symptom characterized by muscle atrophy in a subject, comprising administering to the subject an effective amount of leucine ethyl ester and arginine ethyl ester, or an effective amount of a composition comprising leucine ethyl ester and arginine ethyl ester.

In some embodiments, the disorder characterized by muscle atrophy is selected from the group consisting of aging, bony fractures, weakness, cachexia, denervation, diabetes, dystrophy, exercise-induced skeletal muscle fatigue, fatigue, frailty, immobilization, inflammatory myositis, malnutrition, metabolic syndrome, neuromuscular disease, obesity, post-surgical muscle weakness, post-traumatic muscle weakness, sarcopenia, and toxin exposure.

In some embodiments, the subject is a mammal. In some embodiments, the subject is a human.

In some embodiments, muscle atrophy is muscle atrophy caused by immobilization. In some embodiments, immobilization is caused by bed rest and/or by casting.

In some embodiments, the composition comprises a pharmaceutically acceptable carrier, excipient or diluent. In some embodiments, the composition is administered orally, enterally, or parenterally. In some embodiments, the composition is selected from the group consisting of a food composition, a dietary supplement, a nutritional composition, a nutraceutical, a powdered nutritional product to be reconstituted in water or milk before consumption, a food additive, a medicament, a drink, and a pet food. In some embodiments, the food composition does not include a dietary source of leucine, arginine, or lysine, milk, milk protein, whey protein, whey protein isolate, soy protein, soy protein isolate, casein, or a caseinate. In some embodiments, composition comprises an additional active agent.

In some embodiments, the additional active agent exerts an effect selected from the group consisting of increasing skeletal muscle mass, promoting skeletal muscle recovery, promoting skeletal muscle anabolism, treating or preventing muscle atrophy, and combinations thereof.

In an aspect, the present invention provides a composition comprising a leucine mimetic, an arginine mimetic, and a lysine mimetic. In some embodiments, the leucine mimetic is not the native amino acid leucine. In some embodiments, the arginine mimetic is not the native amino acid arginine. In some embodiments, the lysine mimetic is not the native amino acid lysine. In some embodiments, at least one of the leucine mimetic, arginine mimetic, and lysine mimetic is not a naturally occurring source of leucine, arginine, or lysine, respectively. In some embodiments, at least one of the leucine mimetic, arginine mimetic, and lysine mimetic is isolated and/or purified. In some embodiments, at least one of the leucine mimetic, arginine mimetic, and lysine mimetic is a derivative, analog, metabolite, or byproduct of metabolism of leucine, arginine, and lysine, respectively.

In an aspect, the present invention provides a composition comprising a combination of agents selected from the group consisting of: leucine, a metabolite of leucine, or a source of leucine; arginine, a metabolite of arginine, or a source of arginine; and lysine, a metabolite of lysine, or a source lysine.

In some embodiments, at least one of the combination of agents selected from the group consisting of: leucine, a metabolite of leucine, or a source of leucine; arginine, a metabolite of arginine, or a source of arginine; and lysine, a metabolite of lysine, or a source lysine is isolated and/or purified. In some embodiments, at least one of the sources of leucine, arginine and lysine are not naturally occurring sources. In some embodiments, at least one of the sources of leucine, arginine, or lysine are isolated and/or purified sources of leucine, arginine, or lysine. In some embodiments, at least one of the sources of leucine, arginine, or lysine are carboxy derivatives of leucine, arginine, or lysine. In some at least one of the sources of leucine, arginine, or lysine are carboxy alkyl derivatives of leucine, arginine, or lysine. In some embodiments, all of the L, R, and K comprise carboxy ester derivatives of leucine, arginine, and lysine. In some embodiments, at least two of the lysine, arginine, and lysine comprise carboxy ester derivatives. In some embodiments, the carboxy ester derivatives are selected from the group consisting of methyl esters and ethyl esters. In some embodiments, the composition comprises a leucine carboxy ester, arginine, and a lysine carboxy ester. In some embodiments, the composition comprises leucine methyl ester, arginine, and lysine methyl ester. In some embodiments, the composition comprises leucine ethyl ester, arginine, and lysine methyl ester. In some embodiments, the composition comprises leucine methyl ester, arginine, and lysine ethyl ester. In some embodiments, the composition comprises leucine ethyl ester, arginine, and lysine ethyl ester. In some embodiments, the composition comprises at least two carboxy ester derivatives and one native amino acid selected from the group consisting of leucine, arginine, and lysine.

In some embodiments, the native amino acid selected from the group consisting of leucine, arginine, and lysine is isolated and/or purified.

In some embodiments, the at least two carboxy ester derivative amino acids are carboxy ester derivatives of leucine and lysine. In some embodiments, the native amino acid is arginine. In some embodiments, the carboxy ester derivatives of leucine and lysine are selected from the group consisting of methyl esters, and ethyl esters.

In an aspect, the present invention provides a composition comprising leucine ethyl ester and arginine ethyl ester.

In an aspect, the present invention provides a method of treating or preventing a ribosomopathy in a subject in need thereof, comprising admin administering to the subject an effective amount of a leucine mimetic, an arginine mimetic, or a lysine mimetic, or a composition comprising the leucine mimetic, an arginine mimetic or a lysine mimetic.

In an aspect, the present invention provides a method of treating or preventing a ribosomopathy in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic.

In an aspect, the present invention provides a method of treating or preventing a ribosomopathy in a subject in need thereof comprises administering to the subject an effective amount of a leucine mimetic, an arginine mimetic, and a lysine mimetic, or a composition comprising the leucine mimetic, an arginine mimetic and the lysine mimetic.

In an aspect, the present invention provides a method of treating or preventing a ribosomopathy in a subject in need thereof comprising administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky.

In an aspect, the present invention provides a method of treating or preventing a ribosomopathy in a subject in need thereof comprising administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl.

In an aspect, the present invention provides a method of treating or preventing a ribosomopathy in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl.

In some embodiments, the ribosomopathy is selected from the group consisting of Diamond-Blackfan anemia, 5q-syndrome, Shwachman-Diamond syndrome, X-linked dyskeratosis, cartilage hair hypoplasia, and Treacher Collins syndrome.

In an aspect, the present invention provides a method of preventing autophagy in a subject in need thereof, the method comprising administering to the subject an effective amount of a leucine mimetic, an arginine mimetic, or a lysine mimetic, or a composition comprising the leucine mimetic, an arginine mimetic or a lysine mimetic.

In an aspect, the present invention provides a method of preventing autophagy in a subject in need thereof, the method comprising administering to the subject an effective amount of at least two of a leucine mimetic, an arginine mimetic, and a lysine mimetic, or a composition comprising the at least two of the leucine mimetic, an arginine mimetic, and the lysine mimetic.

In an aspect, the present invention provides a method of preventing autophagy in a subject in need thereof, the method comprising administering to the subject an effective amount of a leucine mimetic, an arginine mimetic, and a lysine mimetic, or a composition comprising the leucine mimetic, an arginine mimetic and the lysine mimetic.

In an aspect, the present invention provides a method of preventing autophagy in a subject in need thereof, the method comprising administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky.

In an aspect, the present invention provides a method of preventing autophagy in a subject in need thereof, the method comprising administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl.

In an aspect, the present invention provides a method of preventing autophagy in a subject in need thereof, the method comprising administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl.

In some embodiments, the subject has been diagnosed with cancer. In some embodiments, the cancer is associated with expression of an oncogenic Ras gene.

In an aspect, the present invention provides a method of inducing satiety in a subject in need thereof, the method comprising administering to the subject an effective amount of a leucine mimetic, an arginine mimetic, or a lysine mimetic, or a composition comprising the leucine mimetic, an arginine mimetic or a lysine mimetic.

In an aspect, the present invention provides a method of inducing satiety in a subject in need thereof, the method comprising administering to the subject an effective amount of at least two of a leucine mimetic, an arginine mimetic, and a lysine mimetic, or a composition comprising the at least two of the leucine mimetic, an arginine mimetic, and the lysine mimetic.

In an aspect, the present invention provides a method of inducing satiety in a subject in need thereof, the method comprising administering to the subject an effective amount of a leucine mimetic, an arginine mimetic, and a lysine mimetic, or a composition comprising the leucine mimetic, an arginine mimetic and the lysine mimetic.

In an aspect, the present invention provides a method of inducing satiety in a subject in need thereof, the method comprising administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky.

In an aspect, the present invention provides a method of inducing satiety in a subject in need thereof comprising administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl.

In an aspect, the present invention provides a method of inducing satiety in a subject in need thereof, the method comprising administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl.

In some embodiments, the subject has, is at risk of developing, or is suspected of having obesity. In some embodiments, the subject has a body mass index of at least 25. In some embodiments, the subject has a body mass index of at least 30.

In an aspect, the present invention provides a method of treating a disease, condition, or disorder which would benefit by activating mTORC1 in a subject, the method comprising administering to the subject an effective amount of a leucine mimetic, an arginine mimetic, or a lysine mimetic, or a composition comprising the leucine mimetic, an arginine mimetic or a lysine mimetic, thereby activating mTORC1 in the subject.

In an aspect, the present invention provides a method of treating a disease, condition, or disorder which would benefit by activating mTORC1 in a subject, the method comprising administering to the subject an effective amount of at least two of a leucine mimetic, an arginine mimetic, and a lysine mimetic, or a composition comprising the at least two of the leucine mimetic, an arginine mimetic, and the lysine mimetic, thereby activating mTORC1 in the subject.

In an aspect, the present invention provides a method of treating a disease, condition, or disorder which would benefit by activating mTORC1 in a subject, the method comprising administering to the subject an effective amount of a leucine mimetic, an arginine mimetic, and a lysine mimetic, or a composition comprising the leucine mimetic, an arginine mimetic and the lysine mimetic, thereby activating mTORC1 in the subject.

In an aspect, the present invention provides a method of treating a disease, condition, or disorder which would benefit by activating mTORC1 in a subject, the method comprising administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky, thereby activating mTORC1 in the subject.

In an aspect, the present invention provides a method of treating a disease, condition, or disorder which would benefit by activating mTORC1 in a subject, the method comprising administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl, thereby activating mTORC1 in the subject.

In an aspect, the present invention provides a method of treating a disease, condition, or disorder which would benefit by activating mTORC1 in a subject, the method comprising administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl, thereby activating mTORC1 in the subject.

In some embodiments, the disease, condition, or disorder is selected from the group consisting of depression, anxiety, chronic stress, a synaptogenesis disorder due to decreased or impaired synaptogenesis, a disorder due to neuronal atrophy, memory deficiency, and a learning deficiency.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™, McKusick-Nathans Institute of Genetic Medicine, Johns Hopkins University (Baltimore, Md.) and National Center for Biotechnology Information, National Library of Medicine (Bethesda, Md.), as of May 1, 2010, World Wide Web URL: http://www.ncbi.nlm.nih.gov/omim/and in Online Mendelian Inheritance in Animals (OMIA), a database of genes, inherited disorders and traits in animal species (other than human and mouse), at http://omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows a Western blot of the phosphorylation levels of mTORC1 substrate S6K achieved in HEK-293T cells upon stimulation with amino acids Leu and Arg, indicating that the combination of amino acids Leu and Arg alone is not sufficient to induce mTORC1 activation to the level achieved upon stimulation with total amino acids at 1×RPMI levels, as measured by the phospho-S6K levels.

FIG. 2 shows a Western blot of the phosphorylation levels of S6K achieved in HEK-293T cells upon stimulation with 1× essential amino acids (EAA) and 1× non-essential amino acids (NEAA), illustrating that 1×EAAs activated mTORC1 to the same extent as 1× total amino acids, whereas 1×NEAA had no activating effect whatsoever.

FIGS. 3A and 3B show Western blots of the phosphorylation levels of S6K achieved in HEK-293T cells upon stimulation with triplet amino acid combinations comprising Leu, Arg, and one additional EAA (FIG. 3A), illustrating surprisingly that the combination of Leu, Arg, and Lys (LRK) was sufficient to emulate the stimulatory effect of total amino acids (FIG. 3B).

FIGS. 4A and 4B show Western blots of the phosphorylation levels of S6K achieved in HEK-293T cells upon stimulation with combinations of EAAs that omitted one amino acid at a time, illustrating that the only combinations that failed to completely activate mTORC1 lacked Leu (L), Arg (R), or Lys (K) (FIG. 4A), and omission of no other EAA affected phosphor-S6K levels (FIG. 4B).

FIGS. 5A and 5B show the effects of amino acid esters on mTORC1 activity. Mice were subjected to an overnight fast and subsequently injected with the indicated amino acids or vehicle control and tissues were harvested 30 minutes later. Liver (FIG. 5A) and gastrocnemius (FIG. 5B) were harvested, whole cell protein extracts were prepared, and western blot analysis was performed by loading 15 ug of liver and 30 ug of muscle extract per sample. All antibodies were obtained from Cell Signaling Technologies (#2215, #2217, #2855, #9644).

FIG. 6 shows the effects of amino acid esters on gastrocnemius mass following hindlimb immobilization and recovery. Mice were subjected to immobilization of the left hind limb for 5 days followed by a 5-day recovery period. Each mouse was injected intraperitoneally twice daily at 9 am and 6 pm during the whole immobilization/recovery periods with the indicated amino acid esters or vehicle control. Rapamycin or vehicle control was injected daily where indicated. Following completion of the study both the right and left gastrocnemius were removed and mass of each muscle was recorded. Percent recovery was calculated by comparing the mass of the left immobilized gastrocnemius to that of the contralateral control leg. Standard error is shown, n=5-6 per treatment.

FIGS. 7A, 7B, 7C and 7D show the effects of the indicated amino acid combinations on mTORC1 activation in mice assayed for S6 phosphorylation 30 minutes after IP administration of the indicated amino acids or 45 minutes after refeeding. FIGS. 7A and 7B show western blots of the phosphorylation levels of mTORC1 substrate S6K achieved in mice liver (FIG. 7A) and gastrocnemius (FIG. 7B). FIG. 7C shows tissue staining of phosphorylated S6 S235/236 in skeletal muscle for each of the indicated amino acid combinations. FIG. 7D is a table summarizing the data from the tissue staining experiments illustrated in FIGS. 7A, 7B and 7C.

FIGS. 8A, 8B and 8C are charts classifying various analogs of leucine (L) (FIG. 8A), arginine (R) (FIG. 8B), and lysine (K) (FIG. 8C), as agonists, antagonists, or neither, of mTORC1 activation.

FIGS. 9A, 9B, 9C, 9D, 9E, 9F, 9G and 9H depict the characterization of RagA^{GTP/GTP} mice. FIG. 9A shows western blots for the proteins indicated. FIG. 9B is a bar graph showing RagA mRNA expression. FIG. 9C is a plot illustrating the weights of RagA^{+/+} (n=24), RagA^{GTP/+} (n=52) and RagA^{GTP/GTP} (n=22) mice at birth (data are scatter dots, mean). FIG. 9D are representative photographs of RagA^{+/+}, RagA^{GTP/+} and RagA^{GTP/GTP} GTP neonates. Scale bar, 1 cm. FIG. 9E demonstrates that early suppression of mTORC1 signaling after birth was determined by immunoblotting of protein extracts from liver and heart of RagA^{+/+} (+/+), RagA^{GTP/+} (G/+) and RagA^{GTP/GTP} (G/G) neonates immediately after Caesarean section (0 h) or after 1 h of fasting (1 h fast). FIG. 9F shows western blots of liver and heart extracts from RagA^{+/+}, RagA^{GTP/+} and RagA^{GTP/GTP} neonates fasted for 10 h were analyzed by immunoblotting for the indicated proteins. FIG. 9G is a graph depicting the survival curve of fasted neonates. FIG. 9H is a graph depicting the survival curve of fasted neonates treated with rapamycin. Numbers indicate the median survival for each curve. *P<0.05; **P<0.01; ***P<0.005.

FIGS. 10A, 10B, 10C, 10D, 10E, 10F, 10G and 10H demonstrate a profound glucose homeostasis defect in RagA^{GTP/GTP} mice. FIG. 10A is a line graph illustrating a glycemia drop in RagA^{+/+}, RagA^{GTP/+} and RagA^{GTP/GTP} and recovery in fasted RagA^{+/+} and RagA^{GTP/+} but not in RagA^{GTP/GTP} neonates (+/+: n=5, 18, 4, 5, 9, 8; G/+: n=10, 26, 10, 13, 26, 21; G/G: n=7, 20, 9, 10, 16, 11, for 0, 1, 2, 3, 6 and 10 h, respectively; mean±s.e.m.). FIG. 10B is a bar graph showing that rapamycin significantly increases glycaemia in RagA^{GTP/GTP} fasted for 6 and 10 h (mean±s.e.m.). NS, not significant. FIG. 10C is a graph showing the extension of survival by glucose injections in fasted RagA^{GTP/GTP} GTP neonates (untreated: n=10; glucose: n=5). FIG. 10D is a bar graph showing the normal expression of genes involved in glucose metabolism in neonatal liver (+/+: n=2, G/+: n=5; G/G: n=4; mean±s.e.m.). FIG. 10E are representative electron microscopy images showing abundant glycogen content in RagA^{+/+} and RagA^{GTP/GTP} hepatocytes before fasting (0 h, left upper panel) and more pronounced glycogen depletion after 10 h of fasting (left lower panel) in RagA^{GTP/GTP} neonates, and the quantification of hepatic glycogen content (+/+: n=5, 3, 4, 4; G/+: n=11, 7, 14, 15; G/G: n=6, 4, 4, 6; for 0, 3, 6 and 10 h, respectively; mean±s.e.m.; AU, arbitrary units) (right panels). FIG. 10F is a bar graph showing the partial rescue of hepatic glycogen content in RagA^{GTP/GTP} fasted for 10 h and treated with rapamycin (rapa) (mean±s.e.m.). FIG. 10G is a series of bar graphs illustrating the quantification of neonatal plasma amounts of branched-chain (BCAA) and essential amino acids at birth (left), after 10 h fasting (middle) and after 10 h fasting with rapamycin treatment (right) (n for +/+, G/+ and G/G, respectively: n=4, 5 and 4 for 0 h; n=4, 4 and 3 for 10 h; n=2, 5 and 3 for rapamycin; mean±s.d.). Values are expressed relative to RagA^{+/+} amounts at each time point. FIG. 10H is a line graph showing the extension of survival by injection of a combination of gluconeogenic amino acids (a.a.) in fasted RagA^{GTP/GTP} neonates (untreated: n=10; amino acids: n=8). *P<0.05; **P<0.01; ns, P>0.05.

FIGS. 11A, 11B, 11C, 11D, 11E and 11F demonstrate impaired autophagy in RagA^{GTP/GTP} neonates. FIG. 11A (top) are representative micrographs of autophagosomes and autophagolysosomes in hepatocytes from RagA^{+/+} neonates fasted for 1 h. Typical autophagosome with a double limiting membrane (arrows); autophagosome and several autolysosomes (arrowheads). Scale bars, 5 μm. FIG. 11A (bottom) is a graph showing the frequency of the two types of organelle (early: autophagosomes; late: autophagolysosomes) detected in cell profiles of hepatocytes and skeletal myocytes from RagA^{+/+} and RagA^{GTP/GTP} neonates. FIG. 11B is a western blot depicting protein extracts from livers of neonates at Caesarean section (0 h) and fasted for 10 h, which were immunoblotted for autophagy markers LC3B and p62. FIG. 11C shows the results of an experiment in which protein extracts from skeletal muscle and heart from neonates at Caesarean section (0 h), fasted for 1 and 2 h, were immunoblotted for indicated proteins. FIG. 11D is a western blot demonstrating the triggering of autophagy by amino-acid withdrawal in MEFs. FIG. 11E show fluorescent microscopy images of recombinant LC3B-GFP expressed in RagA^{+/+} and RagA^{GTP/GTP} MEFs and LC3B localization, in the presence and absence of amino acids, monitored by fluorescence microscopy. LC3B-GFP (green fluorescent protein) clustering, indicative of autophagy, was observed in amino acid-starved RagA^{+/+} but not RagA^{GTP/GTP} MEFs. Scale bar, 10 μm. FIG. 11F shows the localization of recombinant TFEB-GFP determined in MEFs as in FIG. 11E. Nuclear (active) TFEB was observed in RagA^{+/+} MEFs upon amino-acid withdrawal.

FIGS. 12A, 12B, 12C, 12D, 12E, 12F, and 12G demonstrate that the Rag GTPases mediate inhibition of mTORC1 by glucose deprivation. FIG. 12A shows Western blots of whole-cell extracts immunoblotted for the indicated proteins. FIG. 12B shows western blots of whole-cell lysates immunoblotted for the indicated proteins FIG. 12C is a bar graph illustrating that RagA^{+/+} and RagA^{GTP/GTP} immortalized MEFs were deprived of glucose or amino acids and surviving cells quantified in triplicate after 48 h. Cell number is indicated relative to cell number at the start of the treatment; mean±s.d.; ***P<0.005. FIG. 12D shows immunostains depicting mTOR localization as detected by immunofluorescence. HEK-293T-RagB^{GTP} cells show mTOR localized at the lysosomal surface, regardless of glucose concentrations. Scale bars, 10 μm. FIG. 12E shows western blots of protein extracts and immunoprecipitates immunoblotted for the indicated proteins. Glucose and amino acids affect the binding of the v-ATPase to the Ragulator complex. FIG. 12F shows the results of an experiment in which RagA^{+/+} and RagA^{GTP/GTP} primary MEFs were cultured for 1 h in media with the glucose and amino-acid concentrations measured in neonates at birth (0 h) or after fasting for 1 h (1 h) and whole-cell protein extracts were analysed by immunoblotting. FIG. 12G is a diagrammatic illustration showing the proposed model for constitutive RagA-induced neonatal lethality. Green and red boxes indicate active and inactive protein or process, respectively.

FIGS. 13A, 13B, 13C, 13D, 13E, 13F, 13G, 13H, 131, and 13J depict the further characterization of RagA^{GTP/GTP} mice. FIG. 13A (top) is a western blot showing the protein levels of RagA, RagB and RagC in tissues and MEFs. Note that significant RagB levels are found only in brain. FIG. 13A (bottom) is an alignment of mouse RagA to RagB proteins, illustrating that except for the n-terminus extension in RagB, identity is ˜98%. FIG. 13B is a diagrammatic illustration depicting the strategy for RagA knock-in construct and recombination, with a Southern blot of ES cells. FIG. 13C is a Western blot demonstrating that MEFs of all genotypes show inhibition of mTORC1 activity upon serum withdrawal. FIG. 13D is a graph showing the results of a 3T3 protocol performed in RagA^{+/+}, RagA^{GTP/+} and RagA^{GTP/GTP} MEFs. FIG. 13E shows representative images of RagA^{+/+}, RagA^{GTP/+} and RagA^{GTP/GTP} E13.5 embryos. FIG. 13F depicts hematoxylin & eosin staining of liver, skin, heart and brain of RagA^{+/+}, RagA^{GTP/+} and RagA^{GTP/GTP} neonates. FIG. 13G (top) is a western blot depicting the lack of inhibition of mTORC1 activity by 1 h fasting in RagA^{GTP/GTP} neonatal skeletal muscle from leg and diaphragm. FIG. 13G (bottom) is a western blot depicting Akt signaling in 1 h fasted neonatal liver and heart. FIG. 13H shows high mTORC1 activity (p-S6) in tissues from RagA^{GTP/GTP} neonates (versus RagA^{+/+} and RagA^{GTP/+} neonates) after 10 h fasting. FIG. 13I is a bar graph illustrating mRNA expression by qtRT-PCR in livers from RagA^{+/+}, RagA^{GTP/+} and RagA^{GTP/GTP} neonates. FIG. 13J is a western blot showing suppression of mTORC1 activity by rapamycin in liver and heart from RagA^{+/+}, RagA^{GTP/+} and RagA^{GTP/GTP} neonates.

FIGS. 14A, 14B, 14C, and 14D further demonstrate the profound glucose homeostasis defect in RagA^{GTP/GTP} mice. FIG. 14A is a western blot of genes involved in glycogen metabolism. FIG. 14B is a bar graph showing reduced levels of plasma glutamine and alanine in and RagA^{GTP/GTP} neonates fasted for 10 h. FIG. 14C is a bar graph showing similar levels of plasma lactate in RagA^{GTP/GTP} versus RagA^{+/+} and RagA^{GTP/+} neonates fasted 10 h. FIG. 14D is a series of graphs depicting proficient gluconeogenesis in neonates by amino acid substrates, for example, all neonates, regardless of the genotype, undergo a significant increase in glycaemia, reflecting the ability to execute gluconeogenesis from amino acid substrates.

FIGS. 15A, 15B, 15C, 15D and 15E further demonstrate the impaired autophagy in RagA^{GTP/GTP} neonates. FIG. 15A shows representative electron micrographs of RagA^{+/+} livers showing autophagosomes (AP) and autophagolysosomes (AL). Bar indicates 0.5 mm. FIG. 158B is a western blot showing the effect of rapamycin on mTORC1 activity and autophagy markers in neonatal livers fasted for 10 h. FIG. 15C are stains illustrating the immunofluorescence (IF) of endogenous LC3B in RagA^{+/+} and RagA^{GTP/GTP} MEFs starved of amino acids, as done in FIG. 11E for recombinant LC3B. Bar indicates 10 mm. FIG. 15D (top) shows immunofluorescence staining of recombinant TFEB and Lamp2 (as lysosomal marker) in MEFs deprived of amino acids. Bar indicates 10 mm. FIG. 15D (bottom) is a series of graphs illustrating that the TFEB transcriptional program is partially impaired in RagA^{GTP/GTP} MEFs upon amino acid withdrawal. FIG. 15E is a western blot illustrating the triggering of autophagy by fetal bovine serum (FBS) withdrawal in MEFs. Cells were deprived of FBS for the indicated time points, and whole-cell protein extracts were obtained and mTORC1 activity (S6K, 4E-BP1 and ULK-1 phosphorylation) and LC3B determined by immunoblotting.

FIGS. 16A, 16B, 16C, 16D, 16E, 16F, 16G, 16H, 161, 16J and 16K further demonstrate that the Rag GTPases mediate inhibition of mTORC1 by glucose deprivation. FIG. 16A is a bar graph illustrating the decrease in plasma amino acids in all 1 h fasted neonates. FIG. 16B is a western blot depicting the suppression of mTORC1 activity by amino acid deprivation in AMPK-DKO cells. FIG. 16C is a western blot depicting the time course of mTORC1 and AMPK activity in AMPK-wt, AMPK-DKO, RagA^{+/+} and RagA^{GTP/GTP} MEFs deprived of glucose for 0.5, 2 and 4 h. FIG. 16D is a western blot of whole cell lysates immunoblotted for the indicated proteins, showing control HEK-293T cells or those expressing RagB^{GTP} were deprived of growth factors, glucose, amino acids, or glucose and amino acids for 1 h and re-stimulated with glucose and/or amino acids for 10 min. FIG. 16E is a graph illustrating the quantification of the western blots on FIG. 12B and FIG. 16D. FIG. 16F is a western blot showing the effect of amino acid esters on mTORC1 activity in wt MEFs. FIG. 16G is a bar graph showing the quantification of amino acids in RagA^{+/+} and RagA^{GTP/GTP} MEFs deprived of glucose (relative to un-starved controls). FIG. 16H is a western blot showing the effect of aminoimidazole carboxamide ribonucleotide (AICAR) on mTORC1 activity in RagA^{+/+} and RagA^{GTP/GTP} MEFs. FIG. 16I (left) is a western blot showing the effect of amino acids or glucose on mTORC1 activity in GCN2-deficient MEFs. FIG. 16I (right) is a western blot showing the effect of amino acid deprivation on GCN2 activity in RagA^{+/+} and RagA^{GTP/GTP} MEFs. FIG. 16J shows mTOR localization by IF in HEK-293T-RagBGTP and control cells upon amino acid deprivation. FIG. 16K shows mTOR localization by IF in RagA^{+/+} and RagA^{GTP/GTP} MEFs deprived of amino acids or glucose. Bar indicates 10 mm.

FIGS. 17A and 17B demonstrate the effect of leucine or a combination of leucine, arginine and lysine on muscle anabolic signalling in a mouse model of disuse atrophy. FIG. 17A is a western blot showing the phosphorylation levels in mice fasted overnight for 18 hours of ribosomal protein S6 (P-S6 S235/236), 4E-BP1 (P-4E-BP1 (T37/46)) and Akt (P-Akt (S473)) achieved in an immobilized left hindlimb (L*) and a control right hindlimb (R) 30 minutes after an IP injection of vehicle (Fast), 1×L-leucine (Leu), or 1×(L-leucine, L-arginine) plus 0.1×L-lysine (LRK). FIG. 17B is a bar graph depicting the relative levels of P-S6 S235/236 achieved in groups of mice fasted overnight for 18 hours (n=5 Fasted, n=10 Leu and LRK) having an immobilized left hindlimb and a control right hindlimb 30 minutes after an IP injection of vehicle (Fast-R. Fast-L*), 1×L-Leucine (Leu-R, Leu-L*), or 1×(L-Leucine, L-Arginine) plus 0.1×L-Lysine (LRK-R, LRK-L*). 1× is defined as the amount of each amino acid represented in the protein contained in 1 gram of normal chow diet. Standard error is displayed, with one-way ANOVA and Tukey's multiple comparison statistical test applied.

FIGS. 18A and 18B, 18C and 18D depict the effect of various amino acids and amino acid combinations on muscle anabolic signalling in previously fasted (18 hours fasting time) mice. FIG. 18A is a western blot from gastrocnemius muscles measuring levels of various proteins, including phosphorylated ribosomal protein S6 (P-S6 S235/236), 30 minutes after injection with a combination of L-leucine, L-arginine and L-lysine (LRK); a combination of L-leucine ethyl ester (Lee); a combination of L-leucine ethyl ester, L-arginine and L-lysine (LeeRK); a combination of L-leucine, L-citrulline, and L-lysine (LCitK); a combination of L-leucine ethyl ester, L-citrulline, and L-lysine (LeeCK); or refeeding with normal chow. Leucine and arginine were administered at 1×the amount of the respective amino acid represented in the protein contained in 1 gram of normal chow diet. Lysine (K) was provided at 0.1×the amount of lysine present in the protein contained in 1 gram of normal chow. L-Leucine ethyl ester (Lee) was given at 0.5×molar equivalent to 1× the amount of leucine present in the protein contained in 1 gram of normal chow. L-Citrulline was given at 1×molar equivalent to L-Arginine. FIG. 18B is a western blot from gastrocnemius muscles measuring levels of various proteins, including phosphorylated ribosomal protein S6 (P-S6 S235/236), at various times after injection with a combination of the indicated amino acids. “L-O-Et” represents L-Leucine ethyl ester. FIG. 18C is a graph showing the fold increase in leucine and arginine levels in the gastrocnemius muscles analyzed in FIG. 18A. FIG. 18D is a graph showing the fold increase in leucine levels in the gastrocnemius muscles analyzed in FIG. 18B.

FIGS. 19A and 19B depict the effect of administering various combinations of amino acids on plasma amino acid levels following a 24 hours fast in rats. Each figure is a bar graph showing the plasma concentration of leucine, arginine and lysine either 30 minutes (FIG. 19A) or 90 minutes (FIG. 19B) after administering an oral gavage of a vehicle (Vehicle); an equimolar combination of leucine and arginine (LR); an equimolar combination of leucine, arginine and lysine (LRK); or a combination of an equimolar amount of leucine and arginine and a ⅓ molar equivalent of lysine (LR(k)).

FIGS. 20A and 20B depict the effect of administering various combinations of amino acids on levels of phosphorylated 4EBP1 (p4EBP1) in rat gastrocnemius muscle following a 24 hours fast. Each figure is a bar graph showing the fold increase in p4EBP1/total protein ratios in fasted rat gastrocnemius muscle 30 minutes (FIG. 20A) or 90 minutes (FIG. 20B) after administering an oral gavage of a vehicle mimicking fasting (Vehicle); an equimolar combination of leucine and arginine (LR); an equimolar combination of leucine, arginine and lysine (LRK); or a combination of an equimolar amount of leucine and arginine and a ⅓ molar equivalent of lysine (LR(k)). “*” represents a statistically difference from vehicle at P<0.05.

FIGS. 21A, 21B and 21C depict the effect of administering various combinations of amino acids on serum amino acid levels and gastrocnemius muscle p4EBP1 levels. FIG. 21A is a bar graph showing the plasma concentration of leucine, arginine and lysine 30 minutes after administering an oral gavage of a vehicle mimicking fasting (Vehicle); an equimolar combination of leucine, arginine and lysine (LRK); or an equimolar combination of leucine, citrulline and lysine (LCK). FIGS. 21B and 21C are bar graphs showing the fold increase in p4EBP1/total protein ratios in fasted rat gastrocnemius muscle 30 minutes (FIG. 21B) or 90 minutes (FIG. 21C) after administering an oral gavage of a vehicle mimicking fasting (Vehicle); leucine (L); an equimolar combination of leucine, arginine and lysine (LRK); an equimolar combination of leucine, citrulline and lysine (LCK); or a LRK tripeptide.

FIGS. 22A, 22B, 22C and 22D depict the effect of administering various combinations of amino acids or a Leu-Arg-Lys tripeptide on serum amino acid levels and gastrocnemius muscle p4EBP1 levels. FIGS. 22A and 22B are bar graphs showing the plasma concentration of leucine, arginine and lysine 30 minutes (FIG. 22A) or 90 minutes (FIG. 22B) after administering an oral gavage of a vehicle mimicking fasting (Vehicle); an equimolar combination of leucine, arginine and lysine (LRK); or a LRK tripeptide. FIGS. 22C and 22D are bar graphs showing the fold increase in p4EBP1/total protein ratios in fasted rat gastrocnemius muscle 30 minutes (FIG. 22C) or 90 minutes (FIG. 22D) after administering the same agents as in FIGS. 22A and 22B.

FIGS. 23A, 23B, 23C and 23D depict the effect of administering various combinations of amino acids on levels of phosphorylated ribosomal protein S6 (pS6) in gastrocnemius muscle and liver. FIGS. 23A and 23B are bar graphs showing the fold increase in pS6/actin protein ratios in fasted rat gastrocnemius muscle (FIG. 23A) or liver (FIG. 23B) 30 and 60 minutes after administering amino acids via oral gavage. FIGS. 23C and 23D are bar graphs showing the fold increase in pS6/actin protein ratios in fasted rat gastrocnemius muscle (FIG. 23C) or liver (FIG. 23D) 30 and 60 minutes after administering amino acids via intravenous bolus injection.

FIGS. 24A and 24B depict the effect of various amino acid combinations or a Leu-Arg-Lys tripeptide on phosphorylation of an mTORC1 substrate and the rate of muscle protein synthesis in starved rats. FIG. 24A is a bar graph showing the ratio of pS6K/total protein in gastrocnemius muscle 45 minutes following the dosing of a vehicle that mimicked fasting (Vehicle); leucine alone (Leu); an equimolar combination of leucine and arginine (LR); a combination of an equimolar amount of leucine and arginine plus 0.1 molar equivalent of lysine (LRK); a combination of an equimolar amount of leucine and citrulline plus 0.1 molar equivalent of lysine (LCK); or a Leu-Arg-Lys tripeptide (LRK pept) at either 5% or 25% of the normal daily intake in starved rats. FIG. 24B is a bar graph showing the fold increase in rate of muscle protein synthesis in gastrocnemius muscle 45 minutes following the dosing of a vehicle that mimicked fasting (Vehicle); leucine alone (Leu); an equimolar combination of leucine and arginine (LR); a combination of an equimolar amount of leucine and arginine plus 0.1 molar equivalent of lysine (LRK); a combination of an equimolar amount of leucine and citrulline plus 0.1 molar equivalent of lysine (LCK); or a Leu-Arg-Lys tripeptide (LRK pept) at 25% of the normal daily intake in starved rats.

### DETAILED DESCRIPTION OF THE INVENTION

The disclosure relates to methods for modulating mTORC1 activation/activity in cells, organs and in subjects, such as mammals, birds, and fish, and related compositions, kits, and agents for use in those methods. In certain aspects described herein, the disclosure provides methods of increasing skeletal muscle mass (e.g., for body building, to increase meat production in livestock) promoting skeletal muscle anabolism, promoting skeletal muscle recovery (e.g., slowing, halting or reversing muscle atrophy, e.g., immobilization-induced, age-related, space travel-related, undernourishment-related, or inactivity-related muscle atrophy), as well as methods of treating or preventing muscle atrophy, and disorders characterized by muscle atrophy.

In other aspect described herein, the disclosure provides methods for increasing mTORC1 activation in organs, tissues and cells other than skeletal muscle. Such activation has utility in treating ribosomopathies, preventing autophagy, preventing or reversing cardiac muscle atrophy, promoting satiety, preventing depression, treating Birt-Hogg-Dube syndrome, and reducing the effects of jet lag.

The work described herein demonstrates surprising and unexpectedly that, in contrast to reports suggesting that the amino acids leucine or arginine are sufficient to induce mTORC1 activation in certain cell types (see, e.g., Gonzalez et al., “Leucine and arginine regulate trophoblast motility through mTOR-dependent and independent pathways in the preimplantation mouse embryo,” Developmental Biology; 361: 286-300 (2012); Laplante and Sabatini, “mTOR Signaling in Growth Control and Disease,” Cell 149: 274-293 (2012); Hara, et al., “Amino Acid Sufficiency and mTOR Regulate p70 S6 Kinase and eIF-4E BP1 through a Common Effector Mechanism,” J. Biological Chemistry; 273(23): 14484-14494 (1998); and Blommaart, et al., “Phosphorylation of Ribosomal Protein S6 Is Inhibitory for Autophagy in Isolated Rat Hepatocytes,” J. Biological Chemistry; 270(5): 2320-2326 (1995)), the amino acids leucine, arginine, and lysine are required for complete activation of mTORC1. As used herein, “complete activation” refers to the maximal level of mTORC1 activity following replenishment of the full complement of amino acids to an amino acid-deficient media or the maximal level of activity of mTORC1 in response to ad libitum feeding following an overnight fast.

Accordingly, the disclosure contemplates methods of modulating mTORC1 activation or activity in a cell, tissue, or subject.

In one aspect, the disclosure provides a method for modulating mTORC1 activation in a cell, the method comprising modulating the levels of at least one leucine (L) mimetic, at least one arginine (R) mimetic, and at least one lysine (K) mimetic in the cell.

As used herein “modulating” means causing or facilitating a qualitative or quantitative change, alteration, or modification in a molecule, a process, pathway, or phenomenon of interest. Without limitation, such change may be an increase, decrease, a change in binding characteristics, or change in relative strength or activity of different components or branches of the process, pathway, or phenomenon.

The disclosure contemplates modulating mTORC1 activation in a cell by any means that is capable of modulating the at least one L mimetic, the at least one R mimetic, and the at least one K mimetic in the cell. In some contexts, modulating mTORC1 activation in a cell comprises increasing mTORC1 activation and modulating the levels of the at least one L mimetic, the at least one R mimetic, and the at least one K mimetic in the cell comprises increasing the levels of the at least one L mimetic, the at least one R mimetic, and the at least one K mimetic in the cell. Accordingly, in another aspect, the disclosure provides a method of activating mTORC1 in a cell, the method comprising increasing the levels of the at least one L mimetic, the at least one R mimetic, and the at least one K mimetic in the cell.

As used herein “increasing”, “increased”, “increase”, “stimulate”, “enhance” or “activate” are all used herein to generally mean an increase by a statistically significant amount; for the avoidance of any doubt, the terms “increased”, “increase”, “stimulate”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

In some embodiments, the method results in increasing leucine, arginine, and/or lysine beyond the endogenous level of such amino acid available in cells after ingesting a dietary source of the amino acid. In some embodiments, the levels of leucine, arginine, and/or lysine are increased to at least 1.1, at least 1.2, at least 1.3, at least 1.4, at least 1.5, at least 1.6, at least 1.7, at least 1.8, at least 1.9, at least 2.0 or more fold than the endogenous level available in cells after ingesting a dietary source of that amino acid. In some embodiments, where an mTORC1 agonizing arginine mimetic, an mTORC1 agonizing leucine mimetic, and/or an mTORC1 agonizing lysine mimetic is delivered to the cell, the sum of the levels of leucine, arginine, and/or lysine plus the corresponding mTORC1 amino acid mimetic is at least 1.1, at least 1.2, at least 1.3, at least 1.4, at least 1.5, at least 1.6, at least 1.7, at least 1.8, at least 1.9, at least 2.0 or more fold than the endogenous level of leucine, arginine and/or lysine available in cells after ingesting a dietary source of that amino acid. It should be understood that to achieve the aforementioned levels leucine, arginine, lysine, or a corresponding mTORC1 agonizing mimetic thereof can be delivered in the form of either a) one or more single amino acid or mTORC1 mimetics thereof; or b) one or more peptides, non-standard peptides, polypeptides, non-standard polypeptides, proteins or non-standard proteins enriched for one or more those amino acids or mTORC1 mimetics.

The disclosure contemplates the use of any at least one L mimetic, at least one R mimetic, or at least one K mimetic which is capable of stimulating mTORC1 activation alone, or in combination, as measured by phosphorylation of an mTORC1 substrate (e.g., S6K).

As used herein “leucine mimetic” and “L mimetic” are used interchangeably to refer to any agent that either emulates the biological effects of leucine on mTORC1 activation in a cell, as measured by mTORC1 phosphorylation of an mTORC1 substrate (e.g., S6K) in response to the agent, or that increases, directly or indirectly, the level of leucine in a cell. The L mimetic can be any kind of agent. Exemplary L mimetics include, but are not limited to, small organic or inorganic molecules; L-leucine, an mTORC1 agonizing leucine mimetic, saccharides; oligosaccharides; polysaccharides; a biological macromolecule selected from the group consisting of peptides, non-standard peptides, polypeptides, non-standard polypeptides, proteins, non-standard proteins, peptide analogs and derivatives enriched for L-leucine and/or mTORC1 agonizing leucine mimetics; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

The disclosure contemplates methods of identifying leucine mimetics, for example by assessing the ability of a test agent to emulate the biological effects of leucine on mTORC1 activation in a cell, as measured by phosphorylation of an mTORC1 substrate (e.g., S6K). In some embodiments, methods of identifying leucine mimetics include assessing the ability of a test agent to emulate the biological effects of leucine when leucine is used in combination with arginine and lysine to simulate mTORC1 activation in a cell.

The term “mTORC1 agonizing leucine mimetic” as used herein means a mimetic of leucine which, when administered to a subject alone (in the form of a single compound or as part of a non-standard peptide, non-standard polypeptide, or non-standard protein, enriched for such mimetic) or in combination with the other components utilized in the methods of this invention causes an increase in mTORC1 activity in one or more tissues or cells of that subject, as compared to the mTORC1 activity prior to administration of the mimetic. In some embodiments, the subject is determined to be deficient in leucine prior to administration. In some embodiments, an mTORC1 agonizing leucine mimetic causes an increase in mTORC1 activity that is between 50% and 500% of the increase caused by administering an equimolar amount of L-leucine. In some embodiments, an mTORC1 agonizing leucine mimetic causes an increase in mTORC1 activity that is between 80% and 120% of the increase caused by administering an equimolar amount of L-leucine. In some embodiments, an mTORC1 agonizing leucine mimetic causes an increase in mTORC1 activity that is equal to or greater than the increase caused by administering an equimolar amount of L-leucine.

In some embodiments, the L mimetic is not the native amino acid leucine. In some embodiments, the L mimetic is not a naturally occurring source of leucine. In some embodiments, the L mimetic is not a dietary source of leucine.

In some embodiments, the L mimetic comprises the native amino acid leucine. As used herein, “native amino acid” refers to the L-form of the amino acid which naturally occurs in proteins; thus, the term “native amino acid leucine” refers to L-leucine. In some embodiments, the native amino acid leucine is isolated and/or purified.

In some embodiments, the L mimetic comprises the native amino acid methionine (M). In some embodiments, the native amino acid methionine is isolated and/or purified. In some embodiments, methionine can be used, in combination with arginine and lysine, to substitute for and mimic the effects of leucine in skeletal muscle. In some embodiments, methionine can be used, in combination with arginine and lysine, to substitute for and mimic the effects of leucine in liver. In some embodiments, methionine can be used, in combination with arginine and lysine, to substitute for and mimic the effects of leucine in the brain.

In some embodiments, the L mimetic comprises a polypeptide comprising the native amino acid leucine. In some embodiments, the L mimetic comprises a polypeptide comprising a derivative of the native amino acid leucine. In some embodiments, the L mimetic comprises a polypeptide comprising an analog of the native amino acid leucine. In some embodiments, the L mimetic comprises a polypeptide comprising a combination of the native amino acid leucine, a derivative of the native amino acid leucine and/or an analog of the native amino acid leucine. A “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. In some embodiments, a non-standard, naturally occurring amino acid is found in mammals. For example, ornithine, citrulline, and homocysteine are naturally occurring non-standard amino acids that have important roles in mammalian metabolism. Exemplary non-standard amino acids include, e.g., singly or multiply halogenated (e.g., fluorinated) amino acids, D-amino acids, homo-amino acids, N-alkyl amino acids (other than proline), dehydroamino acids, aromatic amino acids (other than histidine, phenylalanine, tyrosine and tryptophan), and α,α disubstituted amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, etc. Modifications may occur anywhere in a polypeptide, e.g., the peptide backbone, the amino acid side-chains and the amino or carboxyl termini. A given polypeptide may contain many types of modifications. Polypeptides may be branched or they may be cyclic, with or without branching. Polypeptides may be conjugated with, encapsulated by, or embedded within a polymer or polymeric matrix, dendrimer, nanoparticle, microparticle, liposome, or the like. Modification may occur prior to or after an amino acid is incorporated into a polypeptide in various embodiments. Polypeptides may, for example, be purified from natural sources, produced in vitro or in vivo in suitable expression systems using recombinant DNA technology (e.g., by recombinant host cells or in transgenic animals or plants), synthesized through chemical means such as conventional solid phase peptide synthesis, and/or methods involving chemical ligation of synthesized peptides (see, e.g., Kent, S., J Pept Sci., 9(9):574-93, 2003 or U.S. Pub. No. 20040115774), or any combination of the foregoing. One of ordinary skill in the art will understand that a protein may be composed of a single amino acid chain or multiple chains associated covalently or noncovalently.

The polypeptide comprising the native amino acid leucine (and/or analogs and/or derivatives of the native amino acid leucine) can be of any length (e.g., 2, 3, 4, 5, 6, 7, 8, 9, 10, 12, 14, 17, 21, 25, 30, 35, 50, 56, 67, 73, 85, 90, 100, 125, 250, 500, 1000, or more residues). In some embodiments, the polypeptide comprising leucine consists entirely of leucine residues. In some embodiments, the polypeptide comprising the native amino acid leucine is polypeptide enriched for leucine residues. In some embodiments, the polypeptide enriched for leucine residues comprises at least 10% content of leucine residues relative to other amino acid residues. In some embodiments, the polypeptide enriched for leucine residues comprises at least 12%, at least 15%, at least 22%, at least 25%, at least 31%, at least 35%, at least 40%, at least 44%, at least 47%, at least 50%, at least 53%, at least 58%, at least 61%, at least 66%, at least 70%, at least 75%, or more content of leucine residues. In some embodiments, the polypeptide enriched for leucine residues comprises at least 80%, at least 85%, at least 90%, at least 91%, at least 92%, at least 93%, at least 94%, at least 95%, at least 96%, at least 97%, at least 98%, at least 99% content of leucine residues. In some embodiments, the polypeptide enriched for leucine residues comprises at least 125 mg of leucine per gram of polypeptide enriched for leucine residues. In some embodiments, the polypeptide enriched for leucine residues comprises at least 130 mg, at least 140 mg, at least 150 mg, at least 160 mg, at least 170 mg, at least 200 mg, at least 225 mg, at least 250 mg of leucine per gram of polypeptide enriched for leucine residues. In some embodiments, the polypeptide enriched for leucine residues comprises at least 250 mg, at least 300 mg, at least 325 mg, at least 350 mg, least 375 mg, at least 400 mg, at least 500 mg, at least 600 mg, at least 700 mg, at least 800 mg, or up to at least 900 mg of leucine per gram of polypeptide.

In some embodiments, the polypeptide comprising leucine is enriched for leucine residues. In some embodiments, the polypeptide enriched for leucine comprises a leucine-rich repeat containing protein or a fragment thereof, such as the leucine rich repeat protein INSP179 described in PCT International Application Publication No. WO/2005/080425. Those skilled in the art will appreciate that a variety of methods exist for obtaining polypeptide comprising and/or enriched for leucine, including, for example, isolating leucine-rich repeats or fragments from polypeptide enriched for leucine, synthetic routes, and recombinant methods (e.g., in vitro transcription and/or translation of nucleic acids comprising leucine codons UUA, UUG, CUU, CUC, CUA, and CUG). Recombinant methods of producing a peptide through the introduction of a vector including nucleic acid encoding the peptide into a suitable host cell is well known in the art, such as is described in Sambrook et al., Molecular Cloning: A Laboratory Manual, 2d Ed, Vols 1 to 8, Cold Spring Harbor, N.Y. (1989); M. W. Pennington and B. M. Dunn, Methods in Molecular Biology: Peptide Synthesis Protocols, Vol 35, Hurnana Press, Totawa, N.J. (1994), contents of both of which are herein incorporated by reference. Peptides can also be chemically synthesized using methods well known in the art. See for example, Merrifield et al., J. Am. Chem. Soc. 85:2149 (1964); Bodanszky, M., Principles of Peptide Synthesis, Springer-Verlag, New York, N.Y. (1984); Kirnrnerlin, T. and Seebach, D. J. Pept. Res. 65:229-260 (2005); Nilsson et al., Annu. Rev. Biophys. Biornol. Struct. (2005) 34:91-118; W. C. Chan and P. D. White (Eds.) Frnoc Solid Phase Peptide Synthesis: A Practical Approach, Oxford University Press, Cary, N.C. (2000); N. L. Benoiton, Chemistry of Peptide Synthesis, CRC Press, Boca Raton, Fla. (2005); J. Jones, Amino Acid and Peptide Synthesis, 2nd Ed, Oxford University Press, Cary, N.C. (2002); and P. Lloyd-Williams, F. Albericio, and E. Giralt, Chemical Approaches to the synthesis of pep tides and proteins, CRC Press, Boca Raton, Fla. (1997), contents of all of which are herein incorporated by reference. Peptide derivatives can also be prepared as described in U.S. Pat. Nos. 4,612,302; 4,853,371; and 4,684,620, and U.S. Pat. App. Pub. No. 2009/0263843, contents of all which are herein incorporated by reference.

In some embodiments, a polypeptide comprising leucine or enriched for leucine is not a whey protein isolate. In some embodiments, a polypeptide comprising lysine or enriched for leucine is not casein or a caseinate. In some embodiments, polypeptide comprising leucine or enriched for leucine is not a soy protein isolate.

In some embodiments, a polypeptide comprising leucine or enriched for leucine is not a dietary source of leucine. As used herein, “dietary source of leucine” refers to a source of leucine in which, prior to ingestion, chewing, or digestion, the leucine is found in its natural state as part of an intact polypeptide within the source (e.g., meats (e.g., chicken, beef, etc.), legumes, grains, vegetables, dairy products (e.g., milk, cheese), eggs, nuts, seeds, seafood, etc.).

In some embodiments, a polypeptide comprising leucine or enriched for leucine does not include any non-essential amino acids. In some embodiments, a polypeptide comprising leucine or enriched for leucine does not include any essential amino acids other than leucine, arginine and lysine. In some embodiments, a polypeptide comprising leucine or enriched for leucine includes at least one non-native form of the amino acid leucine.

In some embodiments, the L mimetic comprises a derivative of the native amino acid leucine. It is contemplated that any derivative of L which activates mTORC1 can be used. L derivatives which activate mTORC1 activation can be readily determined by the skilled artisan according to the teachings disclosed herein (e.g., assaying for L derivatives which increase phosphorylation levels of an mTORC1 substrate (e.g., S6K) either alone, or in combination with the amino acids arginine and lysine or mimetics of arginine or lysine).

In some embodiments, the derivative of L comprises a C-terminus modification to L. As used herein, a “C-terminus modification” refers to the addition of a moiety or substituent group to the amino acid via a linkage between the carboxylic acid group of the amino acid and the moiety or substituent group to be added to the amino acid. The disclosure contemplates any C-terminus modification to L in which L retains the ability to stimulate mTORC1 activation when used alone, or in combination with the amino acids arginine and lysine, as measured by mTORC1 substrate phosphorylation (e.g., S6K). In some embodiments, the C-terminus modification to L comprises a carboxy alkyl of L. In some embodiments, the C-terminus modification to L comprises a carboxy alky ester of L. In some embodiments, the C-terminus modification to L comprises a carboxy alkyl ester. As used herein, the term “alkyl” refers to saturated non-aromatic hydrocarbon chain that may be a straight chain or branched chain, containing the indicated number of carbon atoms (these include without limitation methyl, ethyl, propyl, allyl, or propargyl), which may be optionally inserted with N, O, S, SS, SO₂, C(O), C(O)O, OC(O), C(O)N or NC(O). For example, C₁-C₆ indicates that the group may have from 1 to 6 (inclusive) carbon atoms in it. In some embodiments, the C-terminus modification to L comprises a carboxy alkenyl ester. As used herein, the term “alkenyl” refers to an alkyl that comprises at least one double bond. Exemplary alkenyl groups include, but are not limited to, for example, ethenyl, propenyl, butenyl, I-methyl-2-buten-1-yl and the like. In some embodiments, the C-terminus modification to L comprises a carboxy alkynyl ester. As used herein, the term “alkynyl” refers to an alkyl that comprises at least one triple bond. In some embodiments, the carboxy ester comprises leucine carboxy methyl ester. In some embodiments, the carboxy ester comprises leucine carboxy ethyl ester.

In some embodiments, derivative of L comprises an N-terminus modification to L. As used herein, “N-terminus modification” refers to the addition of a moiety or substituent group to the amino acid via a linkage between the alpha amino group of the amino acid and the moiety or substituent group to be added to the amino acid. The disclosure contemplates any N-terminus modification to L in which the N-terminus modified L retains the ability to stimulate mTORC1 activation alone, or in combination with the amino acids arginine and lysine, as measured by mTORC1 substrate phosphorylation (e.g., S6K).

In some embodiments, the derivative of L comprises L modified by an amino bulky substituent group. As used herein “amino bulky substituent group” refers to a bulky substituent group which is linked to the amino acid via the alpha amino group. The disclosure contemplates the use of any L derivative comprising an amino bulky substituent group that retains its ability to stimulate mTORC1 activation when used alone, or in combination with the amino acids arginine and lysine, as measured by phosphorylation of an mTORC1 substrate (e.g., S6K). An exemplary amino bulky substituent group is a carboxybenzyl (Cbz) protecting group. Accordingly, in some embodiments, the derivative of L comprises L modified by an amino carboxybenzyl (Cbz) protecting group. Other suitable amino bulky substituent groups are apparent to those skilled in the art.

In some embodiments, the derivative of L comprises a side-chain modification to L. As used herein “side-chain modification” refers to the addition of a moiety or substituent group to the side-chain of the amino acid via a linkage (e.g., covalent bond) between the side-chain and the moiety or chemical group to be added. The disclosure contemplates the use of any side-chain modification that permits the side-chain modified amino acid to retain its ability to stimulate mTORC1 activation when used alone, or in combination with the amino acids arginine and lysine or mimetics thereof, as measured by phosphorylation of an mTORC1 substrate (e.g., S6K). An exemplary side-chain modification is a diazirine modification. Accordingly, in some embodiments, the L derivative comprises a photo-crosslinkable L with a diazirine-modified side chain.

In some embodiments, the derivative of L comprises an unnatural amino acid.

In some embodiments, the derivative of L comprises a salt of L.

In some embodiments, the derivative of L comprises a nitrate of L.

In some embodiments, the derivative of L comprises a nitrite of L.

In some embodiments, the L mimetic comprises an analog of the native amino acid leucine. It is contemplated that any analog of L which stimulates mTORC1 activation when used alone, or in combination with the amino acids arginine and lysine, as measured by phosphorylation of an mTORC1 substrate (e.g., S6K) can be used. L analogs which stimulate mTORC1 activation can be readily determined by the skilled artisan according to the teachings disclosed herein (e.g., assaying for L analogs which increase phosphorylation levels of an mTORC1 substrate, e.g., S6K).

Exemplary analogs of L include, but are not limited to, norleucine, threo-L-beta-hydroxyleucine, H-alpha-methyl-D/L-leucine, S-(−)-2-amino-4-pentenoic acid, 3-amino-4-methylpentanoic acid, and leucine-amide hydrochloride (H-Leu-NH₂HCl).

In some embodiments, the L mimetic comprises a metabolite of the native amino acid leucine. It is further contemplated that any metabolite of L that stimulates mTORC1 activation alone or in combination with the amino acids arginine and lysine or mimetics thereof can be used. L derivatives which stimulate mTORC1 activation can be readily determined by the skilled artisan according to the teachings disclosed herein (e.g., assaying for metabolites of L which increase phosphorylation levels of an mTORC1 substrate (e.g., S6K) when used alone, or in combination with arginine and lysine or mimetics thereof. Leucine metabolism has been reviewed by Matthews (JPEN. 1991; 15(3):86S-89S).

Non-limiting examples of metabolites of L that can be used to activate mTORC1 include, but are not limited to, hydroxymethylbutyrate (e.g., β-hydroxy β-methylbutyrate) or a salt or ester thereof (e.g., calcium β-hydroxy β-methylbutyrate), alpha-ketoisocaproate, and β-leucine.

In some embodiments, the L mimetic comprises a metabolite of the native amino acid methionine.

In some embodiments, the L mimetic comprises a byproduct of metabolism of the native amino acid leucine. In some embodiments, the L mimetic comprises an agent which increases endogenous levels of the native amino acid leucine.

In some embodiments, the L mimetic comprises a byproduct of metabolism of the native amino acid methionine.

In some embodiments, the L mimetic is leucine ethyl ester.

As used herein “arginine mimetic” and “R mimetic” are used interchangeably to refer to any agent that either emulates the biological effects of arginine on mTORC1 activation in a cell, as measured by mTORC1 phosphorylation of an mTORC1 substrate (e.g., S6K) in response to the agent, or that increases, directly or indirectly, the level of lysine in a cell. The R mimetic can be any kind of agent. Exemplary R mimetics include, but are not limited to, small organic or inorganic molecules; L-arginine; an mTORC1 agonizing arginine mimetic; saccharides; oligosaccharides; polysaccharides; a biological macromolecule selected from the group consisting of peptides, non-standard peptides, polypeptides, non-standard polypeptides, proteins, non-standard proteins, peptide analogs and derivatives enriched for L-arginine and/or mTORC1 agonizing arginine mimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

The disclosure contemplates methods of identifying arginine mimetics, for example by assessing the ability of a test agent to emulate the biological effects of arginine on mTORC1 activation in a cell, as measured by phosphorylation of an mTORC1 substrate (e.g., S6K). In some embodiments, methods of identifying arginine mimetics include assessing the ability of a test agent to emulate the biological effects of arginine when arginine is used in combination with leucine and lysine to simulate mTORC1 activation in a cell.

The term “mTORC1 agonizing arginine mimetic” as used herein means a mimetic of arginine which, when administered to a subject alone (in the form of a single compound or as part of a non-standard peptide, non-standard polypeptide, or non-standard protein, enriched for such mimetic) or in combination with the other components utilized in the methods of this invention causes an increase in mTORC1 activity in one or more tissues or cells of that subject, as compared to the mTORC1 activity prior to administration of the mimetic. In some embodiments, the subject is determined to be deficient in arginine prior to administration. In some embodiments, an mTORC1 agonizing arginine mimetic causes an increase in mTORC1 activity that is between 50% and 500% of the increase caused by administering an equimolar amount of L-arginine. In some embodiments, an mTORC1 agonizing arginine mimetic causes an increase in mTORC1 activity that is between 80% and 120% of the increase caused by administering an equimolar amount of L-arginine. In some embodiments, an mTORC1 agonizing arginine mimetic causes an increase in mTORC1 activity that is equal to or greater than the increase caused by administering an equimolar amount of L-arginine.

In some embodiments, the R mimetic is not the native amino acid arginine. In some embodiments, the R mimetic is not a naturally occurring source of arginine. In some embodiments, the R mimetic is not a dietary source of arginine.

In some embodiments, the R mimetic comprises the native amino acid arginine. As used herein, the term “native amino acid arginine” refers to L-arginine. In some embodiments, the native amino acid arginine is isolated and/or purified.

In some embodiments, the R mimetic is citrulline.

In some embodiments, the R mimetic comprises a polypeptide comprising the native amino acid arginine. In some embodiments, the R mimetic comprises a polypeptide comprising a derivative of the native amino acid arginine. In some embodiments, the R mimetic comprises polypeptide comprising an analog of the native amino acid arginine. In some embodiments, the R mimetic comprises a polypeptide comprising a combination of the native amino acid arginine, a derivative of the native amino acid arginine and/or an analog of the native amino acid arginine.

The polypeptide comprising the native amino acid arginine (and/or analogs and/or derivatives of the native amino acid arginine) can be of any length (e.g., 2, 3, 4, 5, 6, 7, 8, 9, 10, 12, 14, 17, 21, 25, 30, 35, 50, 56, 67, 73, 85, 90, 100, 125, 250, 500, 1000, or more residues). In some embodiments, the polypeptide comprising arginine consists entirely of arginine residues. In some embodiments, the polypeptide comprising the native amino acid arginine is a polypeptide enriched for arginine residues. In some embodiments, the polypeptide enriched for arginine residues comprises at least 10% content of arginine residues relative to other amino acid residues. In some embodiments, the polypeptide enriched for arginine residues comprises at least 12%, at least 15%, at least 22%, at least 25%, at least 31%, at least 35%, at least 40%, at least 44%, at least 47%, at least 50%, at least 53%, at least 58%, at least 61%, at least 66%, at least 70%, at least 75%, or more content of arginine residues. In some embodiments, the polypeptide enriched for arginine residues comprises at least 80%, at least 85%, at least 90%, at least 91%, at least 92%, at least 93%, at least 94%, at least 95%, at least 96%, at least 97%, at least 98%, at least 99% content of arginine residues. In some embodiments, the polypeptide enriched for arginine residues comprises at least 75 mg of arginine per gram of polypeptide enriched for arginine residues. In some embodiments, the polypeptide enriched for arginine residues comprises at least 80 mg of arginine per gram of polypeptide enriched for arginine residues. In some embodiments, the polypeptide enriched for arginine residues comprises at least 85 mg of arginine per gram of polypeptide enriched for arginine residues. In some embodiments, the polypeptide enriched for arginine residues comprises at least 90 mg of arginine per gram of polypeptide enriched for arginine residues. In some embodiments, the polypeptide enriched for arginine residues comprises at least 95 mg of arginine per gram of polypeptide enriched for arginine residues.

In some embodiments, the polypeptide enriched for arginine residues comprises at least 100 mg of arginine per gram of polypeptide enriched for arginine residues. In some embodiments, the polypeptide enriched for arginine residues comprises at least 110 mg, at least 120 mg, at least 130 mg, at least 140 mg, at least 150 mg, at least 200 mg, at least 225 mg, at least 250 mg of arginine per gram of polypeptide enriched for arginine residues. In some embodiments, the polypeptide enriched for arginine residues comprises at least 250 mg, at least 300 mg, at least 325 mg, at least 350 mg, least 375 mg, at least 400 mg, at least 500 mg, at least 600 mg, at least 700 mg, at least 800 mg, or up to at least 900 mg of arginine per gram of polypeptide.

In some embodiments, a polypeptide enriched for arginine residues is an arginine rich protein or peptide, such as an arginine rich protein described in PCT International Application Publication No. WO/2002/079246. In some embodiments, the polypeptide enriched for arginine residues is enriched for arginine and leucine residues. In some embodiments, the polypeptide enriched for arginine residues comprises a peptide enriched for arginine and lysine, such as the arginine/lysine enriched peptides described in PCT International Application Publication No. WO/2005/007680. In some embodiments, the polypeptide comprising arginine is enriched for arginine, leucine, and lysine residues. Those skilled in the art will appreciate that a variety of methods exist for obtaining polypeptide comprising and/or enriched for arginine, including, for example, isolating arginine rich repeats or fragments from polypeptide enriched for arginine, synthetic routes, and recombinant methods (e.g., in vitro transcription and/or translation of nucleic acids comprising arginine codons CGU, CGC, CGA, CGG, AGA, and AGG).

In some embodiments, a polypeptide comprising arginine or enriched for arginine is not a whey protein or whey protein isolate. In some embodiments, a polypeptide comprising arginine or enriched for arginine is not casein or a caseinate. In some embodiments, a polypeptide comprising arginine or enriched for arginine is not a soy protein isolate.

In some embodiments, a polypeptide comprising arginine or enriched for arginine is not a dietary source of arginine. As used herein, “dietary source of arginine” refers to a source of arginine in which, prior to ingestion, chewing, or digestion, the arginine is found in its natural state as part of an intact polypeptide within the source (e.g., meats (e.g., chicken, beef, etc., legumes, grains, vegetables, dairy products (e.g., milk, cheese), eggs, nuts, seeds, seafood, etc.).

In some embodiments, a polypeptide comprising arginine or enriched for arginine does not include any non-essential amino acids. In some embodiments, a polypeptide comprising arginine or enriched for arginine does not include any essential amino acids other than leucine, arginine and lysine. In some embodiments, a polypeptide comprising arginine or enriched for arginine includes at least one non-native form of the amino acid arginine.

In some embodiments, the R mimetic comprises a derivative of the native amino acid arginine. It is contemplated that any derivative of R which stimulates mTORC1 activation can be used. R derivatives which stimulate mTORC1 activation can be readily determined by the skilled artisan according to the teachings disclosed herein (e.g., assaying for R derivatives which increase phosphorylation levels of an mTORC1 substrate (e.g., S6K) when used alone or in combination with the amino acids leucine and lysine or mimetics of leucine and lysine).

In some embodiments, the derivative of R comprises a C-terminus modification to R. The disclosure contemplates any C-terminus modification to R in which the C-terminus modified R retains the ability to stimulate mTORC1 activation when used alone, or in combination with the amino acids leucine and lysine or mimetics thereof, as measured by mTORC1 substrate phosphorylation (e.g., S6K). In some embodiments, the C-terminus modification to R comprises a carboxy alkyl of R. In some embodiments, the C-terminus modification to R comprises a carboxy ester of R. In some embodiments, the C-terminus modification to R comprises a carboxy alkyl ester. In some embodiments, the C-terminus modification to R comprises a carboxy alkenyl ester. In some embodiments, the C-terminus modification to R comprises a carboxy alkynyl ester. In some embodiments, the carboxy ester comprises arginine carboxy methyl ester. In some embodiments, the carboxy ester comprises arginine carboxy ethyl ester.

In some embodiments, derivative of R comprises an N-terminus modification to R. The disclosure contemplates any N-terminus modification to R in which the N-terminus modified R retains the ability to stimulate mTORC1 activation alone, or in combination with the amino acids leucine and lysine or mimetics thereof, as measured by mTORC1 substrate phosphorylation (e.g., S6K).

In some embodiments, the derivative of R comprises R modified by an amino bulky substituent group. The disclosure contemplates the use of any R derivative comprising an amino bulky substituent group that retains its ability to stimulate mTORC1 activation when used alone, or in combination with the amino acids leucine and lysine or mimetics thereof, as measured by phosphorylation of an mTORC1 substrate (e.g., S6K). An exemplary amino bulky substituent group is a carboxybenzyl (Cbz) protecting group. Accordingly, in some embodiments, the derivative of R comprises R modified by an amino carboxybenzyl (Cbz) protecting group.

In some embodiments, the derivative of R comprises a side-chain modification to R. The disclosure contemplates the use of any side-chain modification that permits the side-chain modified amino acid to retain its ability to stimulate mTORC1 activation when used alone, or in combination with the amino acids leucine and lysine or mimetics thereof, as measured by phosphorylation of an mTORC1 substrate (e.g., S6K). An exemplary side-chain modification is a diazirine modification. Accordingly, in some embodiments, the R derivative comprises a photo-crosslinkable R with a diazirine-modified side chain.

In some embodiments, the derivative of R comprises an unnatural amino acid (e.g., D-arginine).

In some embodiments, the derivative of R comprises a salt of R.

In some embodiments, the derivative of R comprises a nitrate of R.

In some embodiments, the derivative of R comprises a nitrite of R.

In some embodiments, the R mimetic comprises an analog of the native amino acid arginine. It is contemplated that any analog of R which stimulates mTORC1 activation when used alone, or in combination with the amino acids leucine and lysine, as measured by phosphorylation of an mTORC1 substrate (e.g., S6K) can be used. R analogs which stimulate mTORC1 activation can be readily determined by the skilled artisan according to the teachings disclosed herein (e.g., assaying for R analogs which increase phosphorylation levels of an mTORC1 substrate, e.g., S6K).

Exemplary analogs of R include, but are not limited to, L-homoarginine hydrochloride, N^{ω}-methyl-L-arginine, N^{ω}-amino-L-arginine, and N^{ω}-nitro-L-arginine.

In some embodiments, the R mimetic comprises a metabolite of the native amino acid arginine. It is further contemplated that any metabolite of R that stimulates mTORC1 activation alone or in combination with the amino acids leucine and lysine or mimetics thereof can be used. R metabolites which stimulate mTORC1 activation can be readily determined by the skilled artisan according to the teachings disclosed herein (e.g., assaying for metabolites of R which increase phosphorylation levels of an mTORC1 substrate (e.g., S6K) when used alone, or in combination with leucine and lysine or mimetics thereof.

Arginine metabolism has been reviewed by Morris (Nutr. 2007; 137(6):1602S-1609S), Guoyao & Morris (Biochem. J. 1998; 336:1-17), and Racke & Warnken (The Open Nitric Oxide Journal, 2010; 2: 9-19). Non-limiting examples of metabolites of R include, but are not limited to, agmatine, nitric oxide, creatine, polyamines, and urea.

In some embodiments, the R mimetic comprises a byproduct of metabolism of the native amino acid arginine. In some embodiments, the R mimetic comprises an agent which increases endogenous levels of the native amino acid arginine.

As used herein “lysine mimetic” and “K mimetic” are used interchangeably to refer to any agent that either emulates the biological effects of lysine on mTORC1 activation in a cell, as measured by mTORC1 phosphorylation of an mTORC1 substrate (e.g., S6K) in response to the agent, or that increases, directly or indirectly, the level of lysine in a cell. The K mimetic can be any kind of agent. Exemplary K mimetics include, but are not limited to, small organic or inorganic molecules; L-lysine; an mTORC1 agonizing lysine mimetic; saccharides; oligosaccharides; polysaccharides; a biological macromolecule selected from the group consisting of peptides, non-standard peptides, polypeptides, non-standard polypeptides, proteins, non-standard proteins, peptide analogs and derivatives enriched for L-lysine and/or mTORC1 agonizing lysine mimetics; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; and any combination thereof.

The disclosure contemplates methods of identifying lysine mimetics, for example by assessing the ability of a test agent to emulate the biological effects of lysine on mTORC1 activation in a cell, as measured by phosphorylation of an mTORC1 substrate (e.g., S6K). In some embodiments, methods of identifying lysine mimetics include assessing the ability of a test agent to emulate the biological effects of lysine when arginine is used in combination with leucine and arginine to simulate mTORC1 activation in a cell.

The term “mTORC1 agonizing lysine mimetic” as used herein means a mimetic of lysine which, when administered t to a subject alone (in the form of a single compound or as part of a non-standard peptide, non-standard polypeptide, or non-standard protein, enriched for such mimetic) or in combination with the other components utilized in the methods of this invention causes an increase in mTORC1 activity in one or more tissues or cells of that subject, as compared to the mTORC1 activity prior to administration of the mimetic. In some embodiments, the subject is determined to be deficient in lysine prior to administration. In some embodiments, an mTORC1 agonizing lysine mimetic causes an increase in mTORC1 activity that is between 50% and 500% of the increase caused by administering an equimolar amount of L-lysine. In some embodiments, an mTORC1 agonizing lysine mimetic causes an increase in mTORC1 activity that is between 80% and 120% of the increase caused by administering an equimolar amount of L-lysine. In some embodiments, an mTORC1 agonizing lysine mimetic causes an increase in mTORC1 activity that is equal to or greater than the increase caused by administering an equimolar amount of L-lysine.

In some embodiments, the K mimetic is not the native amino acid lysine. In some embodiments, the K mimetic is not a naturally occurring source of lysine. In some embodiments, the K mimetic is not a dietary source of lysine.

In some embodiments, the K mimetic comprises the native amino acid lysine. As used herein, the term “native amino acid lysine” refers to L-lysine. In some embodiments, the native amino acid lysine is isolated and/or purified.

In some embodiments, the K mimetic comprises a polypeptide comprising the native amino acid lysine. In some embodiments, the K mimetic comprises a polypeptide comprising a derivative of the native amino acid lysine. In some embodiments, the K mimetic comprises a polypeptide comprising an analog of the native amino acid arginine. In some embodiments, the k mimetic comprises a polypeptide comprising a combination of the native amino acid lysine, a derivative of the native amino acid lysine, and/or an analog of the native amino acid lysine.

The polypeptide comprising the native amino acid lysine (and/or analogs and/or derivatives of the native amino acid arginine) can be of any length (e.g., 2, 3, 4, 5, 6, 7, 8, 9, 10, 12, 14, 17, 21, 25, 30, 35, 50, 56, 67, 73, 85, 90, 100, 125, 250, 500, 1000, or more residues). In some embodiments, the polypeptide comprising lysine consists entirely of lysine residues. In some embodiments, the polypeptide comprising the native amino acid lysine is a polypeptide enriched for lysine residues. In some embodiments, the polypeptide enriched for lysine residues comprises at least 10% content of lysine residues relative to other amino acid residues. In some embodiments, the polypeptide enriched for lysine residues comprises at least 12%, at least 15%, at least 22%, at least 25%, at least 31%, at least 35%, at least 40%, at least 44%, at least 47%, at least 50%, at least 53%, at least 58%, at least 61%, at least 66%, at least 70%, at least 75%, or more content of lysine residues. In some embodiments, the polypeptide enriched for lysine residues comprises at least 80%, at least 85%, at least 90%, at least 91%, at least 92%, at least 93%, at least 94%, at least 95%, at least 96%, at least 97%, at least 98%, at least 99% content of lysine residues. In some embodiments, the polypeptide enriched for lysine residues comprises at least 100 mg of lysine per gram of polypeptide enriched for lysine residues. In some embodiments, the polypeptide enriched for lysine residues comprises at least 110 mg, at least 120 mg, at least 130 mg, at least 140 mg, at least 150 mg, at least 200 mg, at least 225 mg, at least 250 mg of lysine per gram of polypeptide enriched for lysine residues. In some embodiments, the polypeptide enriched for lysine residues comprises at least 250 mg, at least 300 mg, at least 325 mg, at least 350 mg, least 375 mg, at least 400 mg, at least 500 mg, at least 600 mg, at least 700 mg, at least 800 mg, or up to at least 900 mg of lysine per gram of polypeptide.

In some embodiments, a polypeptide enriched for lysine residues is a lysine rich protein or peptide, such as a lysine rich protein from winged bean described in U.S. Pat. No. 6,184,437. In some embodiments, the polypeptide comprising lysine is a polypeptide enriched for lysine and leucine residues. In some embodiments, the polypeptide comprising lysine is enriched for lysine and arginine, such as the arginine/lysine enriched peptides described in PCT International Application Publication No. WO/2005/007680. In some embodiments, the polypeptide comprising lysine is a polypeptide enriched for lysine, leucine, and arginine residues. Those skilled in the art will appreciate that a variety of methods exist for obtaining polypeptide comprising and/or enriched for lysine, including, for example, isolating lysine rich repeats or fragments from polypeptide enriched for lysine, synthetic routes, and recombinant methods (e.g., in vitro transcription and/or translation of nucleic acids comprising lysine codons AAA or AAG).

In some embodiments, a polypeptide comprising lysine or enriched for lysine is not a whey protein isolate. In some embodiments, a polypeptide comprising lysine or enriched for lysine is not casein or a caseinate. In some embodiments, a polypeptide comprising lysine or enriched for lysine is not a soy protein isolate.

In some embodiments, a polypeptide comprising lysine or enriched for lysine is not a dietary source of lysine. As used herein, “dietary source of lysine” refers to a source of lysine in which, prior to ingestion, chewing, or digestion, lysine is found in its natural state as part of an intact protein within the source (e.g., meats (e.g., chicken, beef, etc., legumes, grains, vegetables, dairy products (e.g., milk, cheese), eggs, nuts, seeds, seafood, etc.).

In some embodiments, a polypeptide comprising lysine or enriched for lysine does not include any non-essential amino acids. In some embodiments, a polypeptide comprising lysine or enriched for lysine does not include any essential amino acids other than leucine, arginine and lysine. In some embodiments, a polypeptide comprising lysine or enriched for lysine includes at least one non-native form of the amino acid arginine.

In some embodiments, the K mimetic comprises a derivative of the native amino acid lysine. It is contemplated that any derivative of K which stimulates mTORC1 activation can be used. K derivatives which stimulate mTORC1 activation can be readily determined by the skilled artisan according to the teachings disclosed herein (e.g., assaying for K derivatives which increase phosphorylation levels of an mTORC1 substrate (e.g., S6K) when used alone or in combination with the amino acids leucine and arginine or mimetics thereof).

In some embodiments, the derivative of K comprises a C-terminus modification to K. The disclosure contemplates any C-terminus modification to K in which the C-terminus modified K retains the ability to stimulate mTORC1 activation when used alone, or in combination with the amino acids leucine and arginine or mimetics thereof, as measured by mTORC1 substrate phosphorylation (e.g., S6K). In some embodiments, the C-terminus modification to K comprises a carboxy alkyl of K. In some embodiments, the C-terminus modification to K comprises a carboxy ester of K. In some embodiments, the C-terminus modification to K comprises a carboxy alkyl ester. In some embodiments, the C-terminus modification to K comprises a carboxy alkenyl ester. In some embodiments, the C-terminus modification to K comprises a carboxy alkynyl ester. In some embodiments, the carboxy ester comprises lysine carboxy methyl ester. In some embodiments, the carboxy ester comprises lysine carboxy ethyl ester.

In some embodiments, derivative of K comprises an N-terminus modification to K. The disclosure contemplates any N-terminus modification to K in which the N-terminus modified K retains the ability to stimulate mTORC1 activation alone, or in combination with the amino acids leucine and arginine or mimetics thereof, as measured by mTORC1 substrate phosphorylation (e.g., S6K).

In some embodiments, the derivative of K comprises K modified by an amino bulky substituent group. The disclosure contemplates the use of any K derivative comprising an amino bulky substituent group that retains its ability to stimulate mTORC1 activation when used alone, or in combination with the amino acids leucine and arginine or mimetics thereof, as measured by phosphorylation of an mTORC1 substrate (e.g., S6K). An exemplary amino bulky substituent group is a carboxybenzyl (Cbz) protecting group. Accordingly, in some embodiments, the derivative of K comprises K modified by an amino carboxybenzyl (Cbz) protecting group.

In some embodiments, the derivative of K comprises a side-chain modification to K. The disclosure contemplates the use of any side-chain modification that permits the side-chain modified amino acid to retain its ability to stimulate mTORC1 activation when used alone, or in combination with the amino acids leucine and arginine or mimetics thereof, as measured by phosphorylation of an mTORC1 substrate (e.g., S6K). An exemplary side-chain modification is a diazirine modification. Accordingly, in some embodiments, the K derivative comprises a photo-crosslinkable K with a diazirine-modified side chain.

In some embodiments, the derivative of K comprises an unnatural amino acid (e.g., D-lysine).

In some embodiments, the derivative of K comprises a salt of K.

In some embodiments, the derivative of K comprises a nitrate of K.

In some embodiments, the derivative of K comprises a nitrite of K.

In some embodiments, the K mimetic comprises an analog of the native amino acid lysine. It is contemplated that any analog of K which stimulates mTORC1 activation when used alone, or in combination with the amino acids leucine and arginine, as measured by phosphorylation of an mTORC1 substrate (e.g., S6K) can be used. K analogs which stimulate mTORC1 activation can be readily determined by the skilled artisan according to the teachings disclosed herein (e.g., assaying for K analogs which increase phosphorylation levels of an mTORC1 substrate, e.g., S6K).

Exemplary analogs of K include, but are not limited to, acetyl-lysine, aminoethyl-cysteine, ε-aminocaproic acid, and omithine.

In some embodiments, the K mimetic comprises a metabolite of the native amino acid lysine. It is further contemplated that any metabolite of K that stimulates mTORC1 activation alone or in combination with the amino acids leucine and arginine or mimetics thereof can be used. K metabolites which stimulate mTORC1 activation can be readily determined by the skilled artisan according to the teachings disclosed herein (e.g., assaying for metabolites of K which increase phosphorylation levels of an mTORC1 substrate (e.g., S6K) when used alone, or in combination with leucine and arginine or mimetics thereof.

Lysine metabolism has been reviewed by Neuberger & Sanger (Biochem J. 1944; 38(1):119-125), Fellows & Lewis (Biochem J. 1973; 136(2):329-334). Non-limiting examples of metabolites of K include, but are not limited to, saccharopine, glutamate, alpha-aminoadipic-6-semialdehyde, and pipecolic acid.

In some embodiments, the K mimetic comprises a byproduct of metabolism of the native amino acid lysine. In some embodiments, the K mimetic comprises an agent which increases endogenous levels of the native amino acid lysine.

In some embodiments, the agent which increases endogenous levels of the native amino acids L, R and K is an agent that prevents the conversion of L, R, or K to another molecule. In some embodiments, the agent that prevents the conversion of L, R, or K to another molecule is a transaminase inhibitor. In some embodiments, the transaminase inhibitor is aminooxyacetic acid.

In some embodiments, the R mimetic is selected from L-arginine; an mTORC1 agonizing arginine mimetic; and a peptide, non-standard peptide, polypeptide, non-standard polypeptide, protein or non-standard protein any of which is enriched for one or both of L-arginine residues or mTORC1 agonizing arginine mimetic residues.

In some embodiments, the L mimetic is selected from L-leucine; an mTORC1 agonizing leucine mimetic; and a peptide, non-standard peptide, polypeptide, non-standard polypeptide, protein or non-standard protein any of which is enriched for one or both of L-leucine residues or mTORC1 agonizing leucine mimetic residues.

In some embodiments, the K mimetic is selected from L-lysine; an mTORC1 agonizing lysine mimetic; and a peptide, non-standard peptide, polypeptide, non-standard polypeptide, protein or non-standard protein any of which is enriched for one or both of L-lysine residues or mTORC1 agonizing lysine mimetic residues.

The term “non-standard” as used herein in reference to a peptide, polypeptide or protein, means that the peptide, polypeptide or protein contains one or more of any of the following: 1) one or more non-proteinogenic amino acids, including one or more mTORC1 agonizing leucine, arginine or lysine mimetic residues; 2) conformational restraints on one or more amino acid; 3) isosteric replacement of one or more amino acid; 4) one or more non-amide bonds; 5) one or more cyclized portions; 6) one or more branched portions; 7) a C-terminal carboxy modification; or 8) an N-terminal amino modification. The term “non-proteinogenic amino acid” means an amino acid not encoded in the genetic code. A specific example of a non-standard peptide, polypeptide or protein is one in which the amide bond between two amino acids is replaced by, for example, a carbon-carbon bond (see, for example Sawyer, in Peptide Based Drug Design, pp. 378-422, ACS, Washington D.C. 1995).

The term “mTORC1 agonizing leucine mimetic residue” as used herein means the divalent or monovalent (if present at a terminus) radical form of an mTORC1 agonizing leucine mimetic present in a non-standard peptide, polypeptide or protein.

The term “mTORC1 agonizing arginine mimetic residue” as used herein means the divalent or monovalent (if present at a terminus) radical form of an mTORC1 agonizing arginine mimetic present in a non-standard peptide, polypeptide or protein.

The term “mTORC1 agonizing lysine mimetic residue” as used herein means the divalent or monovalent (if present at a terminus) radical form of an mTORC1 agonizing lysine mimetic present in a non-standard peptide, polypeptide or protein.

The term “enriched for one or both of L-leucine residue or mTORC1 agonizing leucine mimetic residue” as used herein means that the sum of the number of L-leucine and mTORC1 agonizing leucine residues in a given peptide, polypeptide or protein, or non-standard peptide, polypeptide or protein represents at least 10% of the total residues (amino acid+mimetic residues) in that macromolecule. In some embodiments, the sum of the number of L-leucine and mTORC1 agonizing leucine residues is at least 12%, at least 15%, at least 22%, at least 25%, at least 31%, at least 35%, at least 40%, at least 44%, at least 47%, at least 50%, at least 53%, at least 58%, at least 61%, at least 66%, at least 70%, at least 75%, or more content of leucine residues. In some embodiments, the polypeptide enriched for leucine residues comprises at least 80%, at least 85%, at least 90%, at least 91%, at least 92%, at least 93%, at least 94%, at least 95%, at least 96%, at least 97%, at least 98%, at least 99% content of total residues in the macromolecule.

The term “enriched for one or both of L-arginine residue or mTORC1 agonizing arginine mimetic residue” as used herein means that the sum of the number of L-arginine and mTORC1 agonizing arginine residues in a given peptide, polypeptide or protein, or non-standard peptide, polypeptide or protein represents at least 10% of the total residues (amino acid+mimetic residues) in that macromolecule. In some embodiments, the sum of the number of L-arginine and mTORC1 agonizing arginine residues is at least 12%, at least 15%, at least 22%, at least 25%, at least 31%, at least 35%, at least 40%, at least 44%, at least 47%, at least 50%, at least 53%, at least 58%, at least 61%, at least 66%, at least 70%, at least 75%, or more content of arginine residues. In some embodiments, the polypeptide enriched for arginine residues comprises at least 80%, at least 85%, at least 90%, at least 91%, at least 92%, at least 93%, at least 94%, at least 95%, at least 96%, at least 97%, at least 98%, at least 99% content of total residues in the macromolecule.

The term “enriched for one or both of L-lysine residue or mTORC1 agonizing lysine mimetic residue” as used herein means that the sum of the number of L-lysine and mTORC1 agonizing lysine residues in a given peptide, polypeptide or protein, or non-standard peptide, polypeptide or protein represents at least 10% of the total residues (amino acid+mimetic residues) in that macromolecule. In some embodiments, the sum of the number of L-lysine and mTORC1 agonizing lysine residues is at least 12%, at least 15%, at least 22%, at least 25%, at least 31%, at least 35%, at least 40%, at least 44%, at least 47%, at least 50%, at least 53%, at least 58%, at least 61%, at least 66%, at least 70%, at least 75%, or more content of lysine residues. In some embodiments, the polypeptide enriched for lysine residues comprises at least 80%, at least 85%, at least 90%, at least 91%, at least 92%, at least 93%, at least 94%, at least 95%, at least 96%, at least 97%, at least 98%, at least 99% content of total residues in the macromolecule.

The disclosure contemplates any suitable method of increasing the levels of L-leucine, L-arginine, and/or L-lysine or their corresponding mTORC1 agonizing mimetic in a cell. In some embodiments, this comprises contacting a cell with a leucine mimetic, the arginine mimetic, and the lysine mimetic. In some embodiments, this comprises contacting a cell with one or more peptides, non-standard peptides, polypeptides, non-standard polypeptides, proteins or non-standard proteins enriched for one or more of leucine, arginine, lysine or their corresponding mTORC1 agonizing mimetic. In some embodiments, this comprises contacting a cell with one or more of L-leucine, L-arginine, and/or L-lysine or their corresponding mTORC1 agonizing mimetic. In some embodiments, this comprises contacting a cell with a combination of one or more peptides, non-standard peptides, polypeptides, non-standard polypeptides, proteins or non-standard proteins enriched for one or more of leucine, arginine, lysine or their corresponding mTORC1 agonizing mimetic and one or more of L-leucine, L-arginine, and/or L-lysine or their corresponding mTORC1 agonizing mimetic.

As used herein “contacting the cell” and the like, refers to any means of introducing at least one agent described herein or a composition comprising at least one agent described herein into a target cell in vitro, ex vivo or in vivo, including by chemical and physical means, whether directly or indirectly or whether the at least one agent or the composition comprising the at least one agent physically contacts the cell directly or is introduced into an environment (e.g., culture medium) in which the cell is present or to which the cell is added. It is to be understood that the cells contacted with the at least one agent or composition comprising the at least one agent described herein can also be simultaneously or subsequently contacted with another agent, such as a growth factor or other differentiation agent or environments to stabilize the cells, or to differentiate the cells further. Contacting also is intended to encompass methods of exposing a cell, delivering to a cell, or ‘loading’ a cell with an agent by viral or non-viral vectors, and wherein such agent is bioactive upon delivery.

The method of delivery will be chosen for the particular agent and use (e.g., disorder characterized by muscle atrophy being treated). Parameters that affect delivery, as is known in the art, can include, inter alia, the cell type affected (e.g., myocytes), and cellular location. In some embodiments, “contacting” includes administering the at least one agent (e.g., L mimetic, R mimetic and K mimetic) or a composition comprising the at least one agent to an individual. In some embodiments, “contacting” refers to exposing a cell or an environment in which the cell is located to one or more of a L mimetic, R mimetic, and K mimetic described in the present disclosure. In some embodiments, “contacting” refers to exposing a cell or an environment in which the cell is located to one or more candidate agents of the present disclosure. In some embodiments, the term “contacting” is not intended to include the in vivo exposure of cells to the agents or compositions disclosed herein that may occur naturally (i.e., as a result of digestion of an ordinary meal).

It should be appreciated that the cell can be contacted with the leucine mimetic, the arginine mimetic, and the lysine mimetic together or separately. In one exemplary embodiment, a cell can be contacted with an oligopeptide, a peptide, or polypeptide comprising L, R, and K residues, for example, a synthetic oligopeptide, peptide, or polypeptide containing only L, R, and K residues. In certain exemplary embodiments, disclosed herein is a synthetic oligopeptide, peptide, or polypeptide comprising LRK residues. Such synthetic LRK oligopeptides, peptides, and polypeptides can be of any length (e.g., 2-20 residues, 20-100 residues, 100-1,000 residues, 500-2,000 residues, 1,000-10,000 residues, or longer). The residues comprising such LRK oligopeptides, peptides, or polypeptides can ordered in any fashion, e.g., LRK, LKR, RLK, RKL, KLR, KRL. The residues comprising such LRK oligopeptides, peptides, or polypeptides can also be structured as repeats ordered in any fashion, such as LLL repeats, RRR repeats, KKK repeats, LRK repeats. In certain embodiments, the synthetic LRK oligopeptides, peptides, and polypeptides contains at least 30%, at least 35%, at least 40%, at least 45%, at least 50%, at least 60% or more LRK content. In some embodiments, the synthetic LRK oligopeptides, peptides, and polypeptides contain at least 10% LRK content. In some embodiments, the synthetic LRK oligopeptides, peptides, and polypeptides contain at least 15% LRK content. In some embodiments, the synthetic LRK oligopeptides, peptides, and polypeptides contain at least 20% LRK content. In some embodiments, the synthetic LRK oligopeptides, peptides, and polypeptides contain at least 25% LRK content. In some embodiments, the synthetic LRK oligopeptides, peptides, and polypeptides contain at least 65% LRK content. In some embodiments, the synthetic LRK oligopeptides, peptides, and polypeptides contain at least 70% LRK content. In some embodiments, the synthetic LRK oligopeptides, peptides, and polypeptides contain at least 75% LRK content. In some embodiments, the synthetic LRK oligopeptides, peptides, and polypeptides contain at least 80% LRK content. In some embodiments, the synthetic LRK oligopeptides, peptides, and polypeptides contain at least 85% LRK content. In some embodiments, the synthetic LRK oligopeptides, peptides, and polypeptides contain at least 90% LRK content. In some embodiments, the synthetic LRK oligopeptides, peptides, and polypeptides contain at least 95% LRK content. In some embodiments, the synthetic LRK oligopeptides, peptides, and polypeptides contain 100% LRK content.

In an aspect, the disclosure provides a method of completing activation of mTORC1 in a cell, the method comprising contacting the cell with a leucine mimetic, an arginine mimetic, and a lysine mimetic.

In an aspect, the disclosure provides a method of completing activation of mTORC1 in a cell, the method comprising contacting the cell with leucine (L) or a source thereof, arginine (R) or a source thereof, and lysine (K) or a source thereof, or a composition comprising any of L or the source thereof, R or the source thereof, and K or the source thereof.

In a first particular embodiment, the disclosure provides a method of increasing mTORC1 activity in a subject comprising administering to a subject in need thereof:

a. a first component selected from L-arginine; an mTORC1 agonizing arginine mimetic; and a peptide, non-standard peptide, polypeptide, non-standard polypeptide, protein or non-standard protein any of which is enriched for one or both of L-arginine residues or mTORC1 agonizing arginine mimetic residues;

b. a second component selected from L-leucine; an mTORC1 agonizing leucine mimetic; and a peptide, non-standard peptide, polypeptide, non-standard polypeptide, protein or non-standard protein any of which is enriched for one or both of L-leucine residues or mTORC1 agonizing leucine mimetic residues; and

c. optionally, a third component selected from L-lysine; an mTORC1 agonizing lysine mimetic; and a peptide, non-standard peptide, polypeptide, non-standard polypeptide, protein or non-standard protein any of which is enriched for one or both of L-lysine residues or mTORC1 agonizing lysine mimetic residues; wherein:

each component is present in an acceptable form for administration to the subject;

any two or all three components may be part of a single composition or a single molecule; and

each component is co-administered with one another to the subject.

The term “co-administered”, as used herein means that all components utilized in the methods of this invention may be administered together as part of a single dosage form (such as a single composition of this invention comprising such components) or in two or three (if the third component is utilized) separate dosage forms. Alternatively, each component may be administered prior to, consecutively with, or following the administration of another component utilized in the methods of this invention as long as all components are administered within sufficient time of one another to achieve the desired effect (e.g., increased activation of mTORC1). In such combination therapy treatment, each component is administered by conventional, but not necessarily the same, methods. The administration of a composition comprising two or more components utilized in the methods of this invention does not preclude the separate administration of one or more of the same components to said subject at another time during a course of treatment. In some embodiment, all components that are co-administered are all administered within less than 12 hours of each other. In some embodiment, all components that are co-administered are all administered within less than 8, 6, 4, 3, 2, 1, 0.5, or 0.25 hours of each other. In some embodiments, all components are administered simultaneously (e.g., at the same time) or consecutively (e.g., one right after the other).

In some aspects of the first particular embodiment, the third component is administered to the subject.

In some aspects of the first particular embodiment, at least one of the first component, second component or optional third component is other than a naturally occurring L-form of an amino acid.

In some aspects of the first particular embodiment, the first component is L-arginine or an mTORC1 agonizing arginine mimetic selected from a carboxy terminal modified form of L-arginine and a side-chain modified form of L-arginine. More specifically, the first component is selected from L-arginine, a L-arginine ester,

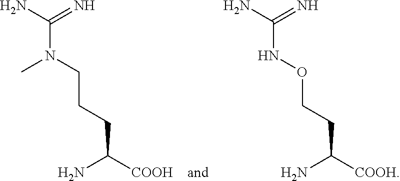

Even more specifically, the L-arginine ester is L-arginine ethyl ester.

In some aspects of the first particular embodiment, the second component is L-leucine or an mTORC1 agonizing leucine mimetic selected from a carboxy terminal modified form of L-leucine, an amino terminal modified form of L-leucine, a side-chain modified form of L-leucine, and L-methionine. In a more specific aspect, the second component is selected from L-leucine, a L-leucine ester, L-methionine

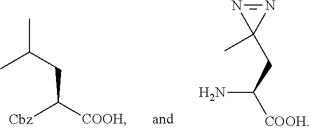

Even more specifically, the L-leucine ester is L-leucine ethyl ester.

In some aspects of the first particular embodiment, the third component, if present, is selected from L-lysine and an L-lysine ester. In some embodiments, the L-lysine ester is an L-lysine ethyl ester.

In yet other aspects of the first particular embodiment, at least one component is selected from:

a. a peptide, non-standard peptide, polypeptide, non-standard polypeptide, protein or non-standard protein any of which is enriched for one or both of L-arginine residues or mTORC1 agonizing arginine mimetic residues;

b. a peptide, non-standard peptide, polypeptide, non-standard polypeptide, protein or non-standard protein any of which is enriched for one or both of L-leucine residues or mTORC1 agonizing leucine mimetic residues; and

c. a peptide, non-standard peptide, polypeptide, non-standard polypeptide, protein or non-standard protein any of which is enriched for one or both of L-lysine residues or mTORC1 agonizing lysine mimetic residues; wherein:

any peptide, non-standard peptide, polypeptide or non-standard polypeptide is optionally associated with one or more of a half-life increasing moiety, a cell penetration moiety, a specific organ-directing moiety, and a specific cell-type-directing moiety.

The term “associated with” as used herein in reference to a half-life increasing moiety, a cell penetration moiety, a specific organ-directing moiety or a specific cell type-directing moiety means such moiety is covalently bound to, conjugated to, cross-linked to, incorporated within (e.g., such as an amino acid sequence within the peptide, polypeptide or protein), or present in the same composition as a peptide, non-standard peptide, polypeptide, non-standard polypeptide, protein or non-standard protein in such a way as to allow such moiety to carry out its function (as defined below). The term “cell penetration moiety” as used herein means a moiety that enhances the ability of the peptide, non-standard peptide, polypeptide, non-standard polypeptide, protein or non-standard protein with which it is associated to penetrate the cell membrane. In some embodiments, the “cell penetration moiety” is an amino acid sequence within the peptide, non-standard peptide, polypeptide, non-standard polypeptide, protein or non-standard protein. Examples of cell penetration sequences include, but are not limited to, Arg-Gly-Asp (RGD), Tat peptide, oligoarginine, MPG peptides, Pep-1, VP22, and Xentry.

The term “specific organ directing moiety” as used herein means a moiety that enhances the ability of the peptide, non-standard peptide, polypeptide, non-standard polypeptide, protein or non-standard protein with which it is associated to be targeted to a specific organ. In some embodiments, the “specific organ directing moiety” is an amino acid sequence, small molecule or antibody that binds to a cell type present in the specific organ. In some embodiments, the “specific organ directing moiety” is an amino acid sequence, small molecule or antibody that binds to a receptor or other protein characteristically present in the specific organ.

The term “specific cell-type directing moiety” as used herein means a moiety that enhances the ability of the peptide, non-standard peptide, polypeptide, non-standard polypeptide, protein or non-standard protein with which it is associated to be targeted to a specific cell type. In some embodiments, the “specific cell-type directing moiety” is an amino acid sequence, small molecule or antibody that binds to a specific receptor or other protein characteristically present in or on the surface of the specific cell type.

In some aspects of the first particular embodiment, at least one component is selected from: a peptide, polypeptide, or protein any of which is enriched for L-arginine residues; a peptide, polypeptide, or protein any of which is enriched for L-leucine residues; and a peptide polypeptide, or protein any of which is enriched for L-lysine residues.

In some aspects of the first particular embodiment, at least two components are independently selected from:

a. a peptide, non-standard peptide, polypeptide, non-standard polypeptide, protein or non-standard protein any of which is enriched for one or both of L-arginine residues or mTORC1 agonizing arginine mimetic residues;

b. a peptide, non-standard peptide, polypeptide, non-standard polypeptide, protein or non-standard protein any of which is enriched for one or both of L-leucine residues or mTORC1 agonizing leucine mimetic residues; and

c. a peptide, non-standard peptide, polypeptide, non-standard polypeptide, protein or non-standard protein any of which is enriched for one or both of L-lysine residues or mTORC1 agonizing lysine mimetic residues.

In a more specific aspect each of the at least two component is independently selected from: a peptide, polypeptide, or protein any of which is enriched for L-arginine residues; a peptide, polypeptide, or protein any of which is enriched for L-leucine residues; and a peptide polypeptide, or protein any of which is enriched for L-lysine residues.

In another aspect of the first particular embodiment, the third component is present and each of the first, second and third components are independently selected from:

a. a peptide, non-standard peptide, polypeptide, non-standard polypeptide, protein or non-standard protein any of which is enriched for one or both of L-arginine residues or mTORC1 agonizing arginine mimetic residues;

b. a peptide, non-standard peptide, polypeptide, non-standard polypeptide, protein or non-standard protein any of which is enriched for one or both of L-leucine residues or mTORC1 agonizing leucine mimetic residues; and

c. a peptide, non-standard peptide, polypeptide, non-standard polypeptide, protein or non-standard protein any of which is enriched for one or both of L-lysine residues or mTORC1 agonizing lysine mimetic residues.

In a more specific aspect, each of the first, second and third components are independently selected from: a peptide, polypeptide, or protein any of which is enriched for L-arginine residues; a peptide, polypeptide, or protein any of which is enriched for L-leucine residues; and a peptide polypeptide, or protein any of which is enriched for L-lysine residues.

In another aspect of the first particular embodiment, at least two components are present on the same peptide, non-standard peptide, polypeptide, non-standard polypeptide, protein or non-standard protein which is enriched for at least two of:

a. L-arginine residues or mTORC1 agonizing arginine mimetic residues;

b. L-leucine residues or mTORC1 agonizing leucine mimetic residues; and

c. L-lysine residues or mTORC1 agonizing lysine mimetic residues.

In a more specific aspect, the at least two components are present on the same peptide, polypeptide, or protein which is enriched for at least two of: L-arginine residues; L-leucine residues; and L-lysine residues.

In still another aspect of the first particular embodiment, each of the first, second and third components is present on the same peptide, non-standard peptide, polypeptide, non-standard polypeptide, protein or non-standard protein which is enriched for
a. L-arginine residues or mTORC1 agonizing arginine mimetic residues;
b. L-leucine residues or mTORC1 agonizing leucine mimetic residues; and
c. L-lysine residues or mTORC1 agonizing lysine mimetic residues.

In a more specific aspect, each of the first, second and third components is present on the same peptide, polypeptide, or protein which is enriched for L-arginine residues, L-leucine residues, and L-lysine residues.

In another aspect of the first particular embodiment, each of the first, second and third components is present on the same peptide, non-standard peptide, polypeptide or non-standard polypeptide, wherein:

a. the peptide, non-standard peptide, polypeptide or non-standard polypeptide consists of residues selected from L-arginine residues, mTORC1 agonizing arginine mimetic residues, L-leucine residues or mTORC1 agonizing leucine mimetic residues, L-lysine residues and mTORC1 agonizing lysine mimetic residues; and

b. the peptide, non-standard peptide, polypeptide or non-standard polypeptide is optionally associated with one or more of a half-life increasing moiety, a cell penetration moiety, a specific organ-directing moiety, and a specific cell-type-directing moiety.

In a more specific aspect every component is present on the same peptide or polypeptide, and wherein the peptide or polypeptide consists of residues selected from L-arginine residues, L-leucine residues and, L-lysine residues; and, optionally, is associated with one or more of a half-life increasing moiety, a cell penetration moiety, a specific organ-directing moiety, and a specific cell-type-directing moiety. In another more specific aspect, every component is present on one peptide or polypeptide, and wherein the peptide or polypeptide comprises at least one mTORC1 agonizing arginine mimetic residue, mTORC1 agonizing leucine mimetic residue, or mTORC1 agonizing lysine mimetic residue; and, optionally is associated with one of more of a half-life increasing moiety, a cell penetration moiety, a specific organ-directing moiety, and a specific cell-type-directing moiety. In an even more specific aspect the at least one mTORC1 agonizing arginine mimetic residue, mTORC1 agonizing leucine mimetic residue, or mTORC1 agonizing lysine mimetic residue is selected from a L-arginine ester,

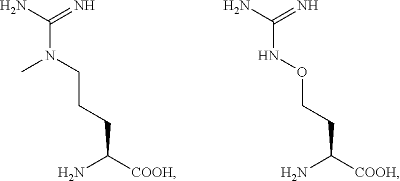

a L-leucine ester, a L-lysine ester, L-methionine,

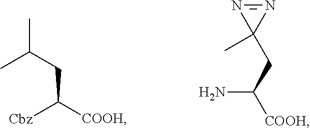

and corresponding monovalent and divalent radicals thereof. In yet another specific aspect the peptide, non-standard peptide, polypeptide or non-standard polypeptide is between two and thirty residues in length, more specifically between two and twelve residues in length.

In yet another aspect of the first particular embodiment, at least one peptide, non-standard peptide, polypeptide, non-standard polypeptide, protein or non-standard protein comprises a cell penetration amino acid sequence.

In any of the above aspects of the first particular embodiment, each of the first, second and third components may be formulated into a pharmaceutically acceptable composition or a nutraceutical composition. Such composition may, for example, be designed for oral, parenteral, intra-muscular or direct to brain administration. In a more specific aspect, at least one of the components is formulated into a controlled release formulation. In another more specific aspect, at least one of the components is formulated into a composition to promote absorption from a specific portion of the digestive tract. In an even more specific aspect, each of the components is formulated into either a controlled release formulation and/or a composition to promote absorption from a specific portion of the digestive tract. In still another more specific aspect, at least one of the components is formulated into a pharmaceutical composition for delivery to a specific organ or cell type (e.g., brain, muscle, fibroblasts, bone, cartilage, liver, lung, breast, skin, bladder, kidney, heart, smooth muscle, adrenal, pituitary, pancreas, melanocytes, blood, adipose, and intestine). It will be understood that formulation for delivery to the brain requires the ability of the active components to cross the blood-brain barrier or to be directly administered to the brain or CNS.

The term “formulated to promote absorption from a specific portion of the digestive tract” as used herein means that at least 50% of all of the active component(s) in such a composition when administered orally are absorbed from a portion of the digestive tract other than the mouth, esophagus, or stomach. In some embodiments at least 60%, 70%, 80%, 90%, 95%, 97% or 99% of all of the active component(s) in such a composition when administered orally are absorbed from a portion of the digestive tract other than the stomach. In some embodiments, the at least 50%, 60%, 70%, 80%, 90%, 95%, 97% or 99% of the components are absorbed from the small intestine. Examples of such formulations include, but are not limited to enteric coatings.

In any of the above aspects of the first particular embodiment, any one or more of the first, second and third components may be formulated into a composition that demonstrates an increase in Cₘₐₓ in the subject as compared to the Cₘₐₓ of a composition consisting of the corresponding component and a pharmaceutically acceptable buffer. Cₘₐₓ is the maximal serum concentration observed in the subject after administration.

In any of the above aspects of the first particular embodiment, any one or more of the first, second and third components may be formulated into a composition that demonstrates an increase in Cₘᵢₙ in the subject as compared to the Cₘᵢₙ of a composition consisting of the corresponding component and a pharmaceutically acceptable buffer. Cₘᵢₙ is the minimal serum concentration observed in the subject after administration.

In any of the above aspects of the first particular embodiment, information about the level of mTORC1 activity in the subject may be received or obtained prior to administration of any of the components.

In any of the above aspects of the first particular embodiment, information about whether the subject is deficient in lysine may be received or obtained. This information may be simply information that the subject is suffering from a condition that is known to cause lysine deficiency. Alternatively, this information may be based on an assay of serum or cellular lysine levels in the patient. In these aspects, the choice of whether or not to administer the third component may be based on such information.

In certain aspects of the first particular embodiment each component is administered to the subject a) prior to retiring for an extended sleep; or b) during sleeping hours. In more specific aspects, each component is administered to the subject within 2 hours, 1 hours, 45 minutes, 30 minutes, 15 minutes, 10 minutes, 5 minutes, 4 minutes, 3 minutes, 2 minute or 1 minute prior to retiring for an extended sleep or during sleeping hours.

In certain aspects of the first particular embodiment each component is administered to the subject only during waking hours. In more specific aspects, each component in administered to the subject just prior to eating a meal. In even more specific aspects, each component is administered to the subject within 2 hours, 1 hours, 45 minutes, 30 minutes, 15 minutes, 10 minutes, 5 minutes, 4 minutes, 3 minutes, 2 minute or 1 minute prior to the time the subject plans to have their first meal after waking from an extended sleep. In other even more specific aspects, each component is administered to the subject during their first meal after waking from an extended sleep or within 1 minute, 2 minutes, 3 minutes, 4 minutes, 5 minutes, 10 minutes, 15 minutes, 30 minutes, 45 minutes, 1 hour, 2 hours, 3 hours, 4 hours or longer after such first meal. In other even more specific aspects, each component is administered to the subject within 2 hours, 1 hours, 45 minutes, 30 minutes, 15 minutes, 10 minutes, 5 minutes, 4 minutes, 3 minutes, 2 minute or 1 minute prior to the time the subject plans to have their second meal after waking from an extended sleep. In other even more specific aspects, each component is administered to the subject during their second meal after waking from an extended sleep or within 1 minute, 2 minutes, 3 minutes, 4 minutes, 5 minutes, 10 minutes, 15 minutes, 30 minutes, 45 minutes, 1 hour, 2 hours, 3 hours, 4 hours or longer after such second meal. In other even more specific aspects, each component is administered to the subject within 2 hours, 1 hours, 45 minutes, 30 minutes, 15 minutes, 10 minutes, 5 minutes, 4 minutes, 3 minutes, 2 minute or 1 minute prior to the time the subject plans to have their third meal after waking from an extended sleep. In other even more specific aspects, each component is administered to the subject during their third meal after waking from an extended sleep or within 1 minute, 2 minutes, 3 minutes, 4 minutes, 5 minutes, 10 minutes, 15 minutes, 30 minutes, 45 minutes, 1 hour, 2 hours, 3 hours, 4 hours or longer after such third meal.

The term “during waking hours” as used herein means at any time during the period from just after the subject awakes from an extended sleep to just before the subject retires for another extended sleep.

The term “during sleeping hours” as used herein means at any time during the period from when the subject retires for an extended sleep to the time the subject awakens from such extended sleep and does not resume such extended sleep after awakening.

The term “extended sleep” as used herein means a period of 3 hours, 4 hours, 5 hours, 6 hours, 7 hours, 8 hours, 9 hours, 10 hours or longer during which time the subject is either asleep, or attempting to fall asleep (either initially or after a brief period of awakening after falling asleep).

In certain aspects of the first particular embodiment each component is administered to a fed subject.

In certain aspects of the first particular embodiment each component is administered to a fasted subject.

The term “fed subject” as used herein means a subject who has ingested a meal within eight hours prior to being administered one of the components utilized in the methods of this invention. In certain embodiments, the “fed subject” has ingested a meal within eight hours prior to being administered all of the components utilized in the methods of this invention. In certain embodiments, the “fed subject” has ingested a meal within 7, 6, 5, 4, 3, 2, or 1 hour prior to being administered all of the components utilized in the methods of this invention.

The term “fasted subject” as used herein means a subject who has not ingested a meal within four hours prior to being administered one of the components utilized in the methods of this invention. In certain embodiments, the “fasted subject” has not ingested a meal within four hours prior to being administered all of the components utilized in the methods of this invention. In certain embodiments, the “fasted subject” has not ingested a meal within two hours prior to being administered all of the components utilized in the methods of this invention. In certain embodiments, the “fasted subject” also does not ingest a meal for a period of 1, 2, 3, 4, 5, 6, 7 or 8 hours after being administered the last of the components utilized in the methods of this invention.

In certain aspects of the first particular embodiment, the subject is selected from a human and a companion animal, more specifically a human, horse, dog or cat, and even more specifically, a human. In other aspects of this first particular embodiment, the subject is selected from livestock or fish.

In other contexts, modulating mTORC1 activation in a cell comprises suppressing mTORC1 activation and modulating the levels of the leucine mimetic, the arginine mimetic, and the lysine mimetic in a cell comprises decreasing the levels of the leucine mimetic, the arginine mimetic, and the lysine mimetic in the cell.

Accordingly, in still another aspect, the disclosure provides a method of suppressing mTORC1 activation in a cell, the method comprising decreasing the levels of a leucine mimetic, an arginine mimetic, and a lysine mimetic in a cell. As used herein “decreasing”, “reduced”, “reduction”, “decrease”, “suppress” or “inhibit” are all used herein generally to mean a decrease by a statistically significant amount. However, for avoidance of doubt, “decreasing”, “reduced”, “reduction” or “decrease”, “suppress” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90%, where the decrease is less than 100%. In some embodiments, the decrease includes a 100% decrease (e.g. absent level as compared to a reference sample), or any decrease between 10-100% as compared to a reference level.

The disclosure contemplates any suitable method of decreasing the levels of a L mimetic, R mimetic, and K mimetic in a cell or any agent which is capable of decreasing the levels of the L mimetic, R mimetic, and K mimetic in the cell. In some embodiments, decreasing the levels of the L mimetic, R mimetic, and K mimetic in the cell comprises depriving the cell of the L mimetic, R mimetic, and K mimetic. Deprivation of amino acid mimetics L, R and K in a cell inhibits phosphorylation of an mTORC1 substrate (e.g., S6K and/or 4EBP1).

Non-limiting in vitro examples of depriving a cell of LRK include, but are not limited to incubating or culturing a cell in the absence of amino acids L, R and K or mimetics thereof, contacting the cell with at least one agent that converts L, R or K to a different amino acid or molecule (e.g., Sylvester et al., describe the conversion of leucine to valine, Biochemistry 1981, 20, 5609-5611), contacting the cell with at least one agent that blocks absorption or uptake of the amino acids L, R, and K into the cell, etc. An example of an agent that deprives a cell of R is an arginine decomposing enzyme, such as an arginine decarboxylase enzyme (biosynthetic arginine decarboxylase of E. coli or a modified version thereof) described in U.S. Pat. No. 6,261,557. Another example of an agent that deprives a cell of R is the arginine consuming agent arginase. An example of an enzyme that deprives the cell of lysine is the enzyme lysine decarboxylase.

In some embodiments, depriving a cell of L, R, and K comprises administering an agent that inhibits amino acid transport of L, R, or K thus inhibiting intestinal uptake and absorption of L, R and K. In some embodiments, an agent that inhibits amino acid transport of L, R, or K is an agent that inhibits cationic amino acid transport (e.g., an inhibitor of the Na+-independent Y(+) system, i.e., a member of the SCL7 family of membrane transporters). In some embodiments, an agent that inhibits amino acid transport of L, R or K is an agent that inhibits Na+ dependent transport. In some embodiments, the agent inhibits the L-type amino acid transporters (LAT). In some embodiments, the agent inhibits a saturable pH-independent transporter.

In some embodiments, the agent inhibits the lysosomal lysine/arginine transporter LAAT-1.

In practicing the subject methods, any cell that expresses mTORC1 can be targeted for modulation. Non-limiting examples of specific cell types in which mTORC1 can be modulated include fibroblast, cells of skeletal tissue (bone (e.g., proliferative and hypertrophic chondrocytes) and cartilage), cells of epithelial tissues (e.g. liver, lung, breast, skin, bladder and kidney), cardiac and smooth muscle cells (e.g., cardiomyocytes), neural cells (glia and neurons), cells of the hypothalamus, hippocampal cells, endocrine cells (adrenal, pituitary, pancreatic islet alpha and beta cells), exocrine pancreatic cells (e.g., acinar cells), melanocytes, many different types of hematopoietic cells (e.g., macrophages, cells of B-cell or T-cell lineage, neutrophils, red blood cells, and their corresponding stem and progenitor cells, lymphoblasts), cells of both white adipose tissue and brown adipose tissue (e.g., adipocytes), and intestinal cells (e.g., Paneth cells, enterocytes, goblet cells). In some embodiments, the cell is a mammalian cell. In some embodiments, the cell is a human cell. In some embodiments, the cell is a HEK293T cell.

### Methods of Treatment

In various aspects, any of the methods of increasing mTORC1 activation/activity set forth herein is used for increasing skeletal muscle mass, promoting skeletal muscle anabolism, improving muscle function, reversing muscle atrophy (e.g., promoting skeletal muscle recovery) or preventing muscle atrophy. In some aspects, the method is used to reverse muscle atrophy or to prevent muscle atrophy due to inactivity, immobilization, or age of the subject or a disease or condition suffered by the subject. This may be due, for example, to a broken bone, a severe burn, a spinal injury, an amputation, a degenerative disease (e.g., muscular dystrophy, Spinal Muscle Atrophy, ALS, sarcopenia, muscle denervation, an inflammatory myopathy and myasthenia gravis), a condition wherein recovery requires bed rest for the subject, a stay in an intensive care unit, long-term hospitalization, a disease or condition known to be associated with cachexia (e.g., cancer, AIDS, SARS, chronic heart failure, COPD, rheumatoid arthritis, liver disease, kidney disease and trauma), a disease or condition known to be associated with malabsorption (e.g., Crohn's disease, irritable bowel syndrome, celiac disease, and cystic fibrosis), malnutrition, recovery from space travel, and during or immediately followed participation in military training or armed conflict. The term “severe burn” as used herein refers to a second or third degree burn or to any type of burn that covers at least 20% of the subject's body.

The term “bed rest” as used herein means that the subject is confined or required by a doctor to remain in bed, sitting and/or lying down for at least 80% of the day for at least 3 days.

The term “long-term hospitalization” as used herein means a stay in a hospital or other health care facility for at least five days.

As used herein, “increasing skeletal muscle mass” refers to a statistically significant increase in the skeletal muscle mass. In some embodiments of various aspects, increasing skeletal muscle mass refers to a reversal of skeletal muscle loss. In some embodiments of various aspects, increasing skeletal muscle mass refers to an increase in skeletal muscle mass of at least 5%, at least 7%, at least 12%, at least 15%, at least 18%, at least 20%, at least 21%, at least 25%, at least 27%, at least 30%, at least 33% or more relative to the skeletal muscle mass prior to contacting the skeletal muscle with the leucine, arginine, and/or lysine mimetics or administering a leucine, arginine, and/or lysine mimetic or a composition comprising the leucine, arginine, and/or lysine mimetic to the subject. In some embodiments, increasing skeletal muscle mass refers to an increase in skeletal muscle mass of a subject to within 35%, within 33%, within 30%, within 28%, within 24%, within 22%, within 18%, within 15%, within 12%, within 10%, within 9%, within 8%, within 7%, within 6%, within at least 5% or more of the skeletal muscle before onset of the disorder, condition, or symptom associated with muscle atrophy, or onset of the muscle atrophy itself.

In some embodiments, any of the methods of increasing mTORC1 activation/activity set forth herein is used for increasing skeletal muscle mass.

In an aspect, the disclosure provides a method of increasing skeletal muscle mass, comprising contacting skeletal muscle or skeletal muscle cells with leucine ethyl ester and arginine ethyl ester, or a composition comprising leucine ethyl ester and arginine ethyl ester. In some embodiments, the leucine ethyl ester and arginine ethyl ester stimulate mTORC1 activation in the skeletal muscle or skeletal muscle cells, thereby promoting skeletal muscle anabolism and increasing skeletal muscle mass.

In an aspect, the disclosure provides a method of increasing skeletal muscle mass, comprising contacting skeletal muscle with leucine ethyl ester and arginine ethyl ester, or a composition comprising leucine ethyl ester and arginine ethyl ester. In some embodiments, the leucine ethyl ester and arginine ethyl ester stimulate mTORC1 activation in the skeletal muscle or skeletal muscle cells, thereby promoting skeletal muscle anabolism and increasing skeletal muscle mass.

Methods of determining whether agents or compositions comprising the agents described herein are effective at increasing skeletal muscle mass involve measuring skeletal muscle mass and are available to the skilled artisan. Exemplary such methods include but are not limited to dual-energy X-ray absorptiometry (DXA) which permits muscle mass estimates from equations using appendicular lean soft tissue measured by DXA, weight and height (see Dorsey et al. Nutrition & Metabolism. 2010; 7:41), and whole body multi-slice magnetic resonance imaging (MRI) (Id.). Other suitable methods are apparent to the skilled artisan.

In an aspect, the disclosure provides a method of increasing skeletal muscle mass in a subject, comprising administering to the subject an effective amount of leucine ethyl ester and arginine ethyl ester, or an effective amount of a composition comprising leucine ethyl ester and arginine ethyl ester. In some embodiments, the leucine ethyl ester and arginine ethyl ester stimulate mTORC1 activation in the subject, thereby promoting skeletal muscle anabolism and increasing the subject's skeletal muscle mass.

In some aspects, the method of increasing skeletal muscle mass is used following exercise.

In some aspects, the method of increasing skeletal muscle mass is used in conjunction with physical therapy, as part of total parenteral nutrition, or to promote functional electrical stimulation.

In some aspects, the method of increasing skeletal muscle mass causes an increase in muscle-to-fat ratio.

In some aspects, the method of increasing skeletal muscle mass is used to increase skeletal muscle mass (or increase the muscle-to fat ratio) in a non-human animal, such as livestock, fish or poultry. In these aspects, each of the components may be administered as an additive to the feed of the non-human animal.

The term “non-human animal” as used herein includes all vertebrates, e.g., non-mammals (such as chickens, amphibians, reptiles) and mammals, such as non-human primates, domesticated and/or agriculturally useful animals, e.g., sheep, dog, cat, cow, pig, etc.

The term “livestock”, as used herein refers to any farmed animal. Preferably, livestock is one or more of ruminants such as cattle (e.g. cows or bulls (including calves)), mono-gastric animals such as poultry (including broilers, chickens and turkeys), pigs (including piglets), birds, or sheep (including lambs). In an aspect, the disclosure provides a method of promoting skeletal muscle anabolism in a subject, comprising administering to the subject an effective amount of leucine ethyl ester and arginine ethyl ester, or an effective amount of a composition comprising leucine ethyl ester and arginine ethyl ester. In some embodiments, the leucine ethyl ester and arginine ethyl ester stimulate mTORC1 activation in the subject's skeletal muscle or skeletal muscle cells, thereby promoting skeletal muscle anabolism in the subject.

Methods of determining whether agents or compositions comprising the agents described herein are effective at promoting skeletal muscle anabolism are available to the skilled artisan. An exemplary method of measuring the effects of a composition described herein can be adapted by the skilled artisan from the teachings of Wilkinson et al. (published online before print, doi: 10.1113/jphysiol.2013.253203). Other suitable methods are apparent to the skilled artisan.

In an aspect, the disclosure provides a method of promoting skeletal muscle recovery in a subject, comprising administering to the subject an effective amount of leucine ethyl ester and arginine ethyl ester, or an effective amount of a composition comprising leucine ethyl ester and arginine ethyl ester. In some embodiments, the leucine ethyl ester and arginine ethyl ester stimulate mTORC1 activation in the skeletal muscle or skeletal muscle cells, thereby promoting skeletal muscle anabolism and increasing skeletal muscle mass.

In an aspect, the disclosure provides a method of promoting skeletal muscle recovery after immobilization-induced muscle atrophy in a subject, comprising administering to the subject an effective amount of leucine ethyl ester and arginine ethyl ester, or an effective amount of a composition comprising leucine ethyl ester and arginine ethyl ester. In some embodiments, the leucine ethyl ester and arginine ethyl ester stimulate mTORC1 activation in the skeletal muscle or skeletal muscle cells, thereby promoting skeletal muscle anabolism, increasing skeletal muscle mass and promoting skeletal muscle recovery after immobilization-induced muscle atrophy in the subject.

Methods of determining whether agents or compositions comprising the agents described herein are effective at promoting skeletal muscle recovery are available to the skilled artisan. Exemplary methods of evaluating skeletal muscle recovery (e.g., regeneration) comprise histological staining, quantitative histology, and measuring contractile properties of muscle post-injury (see, e.g., Baoge et al. ISRN Orthopedics. 2012; Article ID 689012, 7 pages). Other suitable methods are apparent to the skilled artisan.

The compositions and methods described herein are useful for treating or preventing muscle atrophy and disorders, conditions, or symptoms associated with muscle atrophy. Generally, the methods of treating or preventing muscle atrophy and disorders, conditions, or symptoms associated with muscle atrophy involved administering therapeutically effective amounts of agents or compositions comprising agents described herein to a subject in need of such treatment.

As used herein, the phrase “therapeutically effective amount”, “effective amount” or “effective dose” refers to an amount that provides a therapeutic or aesthetic benefit in the treatment, prevention, or management of, for example, muscle atrophy, e.g. an amount that provides a statistically significant decrease in at least one symptom, sign, or marker of muscle atrophy.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

In an aspect, the present disclosure provides a method of treating or preventing muscle atrophy in a subject in need thereof, comprising administering to the subject a therapeutically effective amount of a leucine mimetic, an arginine mimetic, and a lysine mimetic, or a therapeutically effective amount of a composition comprising the L mimetic, the R mimetic, or the K mimetic.

In an aspect, the present disclosure provides a method of treating or preventing muscle atrophy in a subject in need thereof, comprising administering to the subject a therapeutically effective amount of L, a metabolite of L, or a source of L, R, a metabolite of R, or a source of R, and K, a metabolite of K, or a source of K, or a therapeutically effective amount of a composition comprising L, a metabolite of L, or a source of L, R, a metabolite of R, or a source of R, and K, a metabolite of K, or a source of K.

In an aspect, the disclosure provides a method of treating or preventing muscle atrophy in a subject, comprising administering to the subject an effective amount of leucine ethyl ester and arginine ethyl ester, or an effective amount of a composition comprising leucine ethyl ester and arginine ethyl ester.

In an aspect, the disclosure provides a method of treating or preventing a disorder, condition, or symptom characterized by muscle atrophy in a subject, comprising administering to the subject an effective amount of leucine ethyl ester and arginine ethyl ester, or an effective amount of a composition comprising leucine ethyl ester and arginine ethyl ester. Exemplary disorders, conditions, or symptoms characterized by muscle atrophy include, but are not limited to, aging, bony fractures, weakness, cachexia, denervation, diabetes, dystrophy, exercise-induced skeletal muscle fatigue, fatigue, frailty, immobilization, inflammatory myositis, malnutrition, metabolic syndrome, neuromuscular disease, obesity, post-surgical muscle weakness, post-traumatic muscle weakness, sarcopenia, and toxin exposure.

In certain aspects, the methods of increasing mTORC1 activity/activation disclosed herein are used for treating Birt-Hogg-Dube syndrome. Birt-Hogg-Dube syndrome (OMIM#135150; also known as Hornstein-Knickenberg syndrome and Fibrofolliculomas with trichodiscomas and acrochordons) is an autosomal dominant genodermatosis marked by hair follicle hamartomas (manifesting as disfiguring facial folliculomas), kidney tumors, and spontaneous pneumothorax. Without wishing to be bound by theory, it is believed that increasing mTORC1 activity/activation can be useful in the treatment of Birt-Hogg-Dube syndrome, for example, to ameliorate (e.g., reduce or arrest the growth or progression of) the disfiguring facial folliculomas. Accordingly, the inventors believe that compositions comprising amino acid mimetics L, R and K may be useful for treating Birt-Hogg-Dube syndrome.

In an embodiment, a method of treating or preventing Birt-Hogg-Dube syndrome in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of treating or preventing Birt-Hogg-Dube syndrome in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of treating or preventing Birt-Hogg-Dube syndrome in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of treating or preventing Birt-Hogg-Dube syndrome in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of treating or preventing Birt-Hogg-Dube syndrome in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of treating or preventing Birt-Hogg-Dube syndrome in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

In certain aspects, the methods of increasing mTORC1 activity/activation disclosed herein are used for treating ribosomopathies. Ribosomopathies are disorders where genetic abnormalities lead to impairments in ribosome biogenesis and function, which result in distinct clinical phenotypes. Ribosomopathies have been reviewed by Narla and Ebert (Blood, 2010; 115(16):3196-3205). Recent reports indicate that the amino acid leucine can successfully be used to treat various ribosomopathies. For example, Pospisilova et al. report the successful treatment of a Diamond-Blackfan anemia patient using the amino acid leucine (Haematologica. 2007; 92(5):e66-7); Yip et al. describe the involvement of L-leucine in activation of mTOR signaling in 5q-syndrome, as well as RPS14-deficient erythroblasts (Leukemia. 2013; doi: 10.1038/leu.2013.20 Epub ahead of print); and Payne et al. report that L-leucine enhances the developmental and anemic defects of Diamond-Blackfan anemia and del(5q) MDS through mTOR pathway activation (Blood. 2012; 120(11):2214-24). Accordingly, without wishing to be bound by theory, the inventors believe that compositions comprising amino acid mimetics L, R and K may be useful for treating ribosomopathies.

The disclosure contemplates treating any of the ribosomopathies using a leucine mimetic, an arginine mimetic and a lysine mimetic or a composition comprising the leucine, arginine, and lysine mimetics. Exemplary ribosomopathies which can be treated include, but are not limited to Diamond-Blackfan anemia, Schwachman-Diamond syndrome, dyskeratosis congenita, cartilage hair hypoplasia, Treacher Collins syndrome, and 5q-syndrome.

The disclosure contemplates methods of treating or preventing Diamond-Blackfan anemia. In an embodiment, a method of treating or preventing Diamond-Blackfan anemia in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of treating or preventing Diamond-Blackfan anemia in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of treating or preventing Diamond-Blackfan anemia in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of treating or preventing Diamond-Blackfan anemia in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of treating or preventing Diamond-Blackfan anemia in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of treating or preventing Diamond-Blackfan anemia in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

Diamond-Blackfan anemia involves a genetic defect in any of the RPS19, RPS24, RPS17, RPL35A, RPL5, RPL11, RPS7, RPL36, RPS15, and RPS27A genes and is marked by anemia, macrocytosis, reticulocytopenia, and a decrease or absence of erythroid precursors in generally normocellular bone marrow. A subject is diagnosed with Diamond-Blackfan anemia, typically within the first year of life, when presenting with symptoms of pallor and lethargy. In many instances, subjects have a family history of the disease. Testing for elevated red blood cell adenosine deaminase levels, the presence of fetal membrane antigen “i,” as well as physical abnormalities such as short stature, thumb abnormalities, and cardiac defects can also be used to diagnose a subject with Diamond-Blackfan anemia. In some embodiments, the method includes diagnosing a subject with Diamond-Blackfan anemia. In some embodiments, the method further includes selecting a subject who has been diagnosed with Diamond-Blackfan anemia, e.g., for treatment with a L mimetic, an R mimetic or a K mimetic, or a combination thereof.

The disclosure contemplates methods of treating or preventing 5q-syndrome. In an embodiment, a method of treating or preventing 5q-syndrome in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of treating or preventing 5q-syndrome in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of treating or preventing 5q-syndrome in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of treating or preventing 5q-syndrome in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of treating or preventing 5q-syndrome in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of treating or preventing 5q-syndrome in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

5q-syndrome involves a genetic defect in the RPS14 gene and is marked by interstitial deletion of the long arm of chromosome 5, severe macrocytic anemia, and normal/elevated platelets exhibiting hypolobulated micromegakaryocytes. A subject is diagnosed with 5q-syndrome via bone marrow aspiration or biopsy with karyotyping. In some embodiments, the method includes diagnosing a subject with 5q-syndrome. In some embodiments, the method further includes selecting a subject who has been diagnosed with 5q-syndrome, e.g., for treatment with a L mimetic, an R mimetic or a K mimetic, or a combination thereof.

The disclosure contemplates methods of treating or preventing Shwachman-Diamond syndrome. In an embodiment, a method of treating or preventing Shwachman-Diamond syndrome in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of treating or preventing Shwachman-Diamond syndrome in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of treating or preventing Shwachman-Diamond syndrome in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of treating or preventing Shwachman-Diamond syndrome in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of treating or preventing Shwachman-Diamond syndrome in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of treating or preventing Shwachman-Diamond syndrome in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

Shwachman-Diamond syndrome involves defect in the SBDS gene and is marked by neutropenia, infections, pancreatic insufficiency, and short stature. A subject is diagnosed with Shwachman-Diamond syndrome via SBDS gene testing. In some embodiments, the method includes diagnosing a subject with Shwachman-Diamond syndrome. In some embodiments, the method further includes selecting a subject who has been diagnosed with Shwachman-Diamond syndrome, e.g., for treatment with a L mimetic, an R mimetic or a K mimetic, or a combination thereof.

The disclosure contemplates methods of treating or preventing X-linked dyskeratosis. In an embodiment, a method of treating or preventing X-linked dyskeratosis in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of treating or preventing X-linked dyskeratosis in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of treating or preventing X-linked dyskeratosis in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of treating or preventing X-linked dyskeratosis in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of treating or preventing X-linked dyskeratosis in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of treating or preventing X-linked dyskeratosis in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

X-linked dyskeratosis involves a genetic defect in the DKC1 gene and is marked by cytopenias, skin hyperpigmentation, nail dystrophy, and oral leukoplakia. A subject is diagnosed with X-linked dyskeratosis via telomere length analysis. In some embodiments, the method includes diagnosing a subject with X-linked dyskeratosis. In some embodiments, the method further includes selecting a subject who has been diagnosed with X-linked dyskeratosis, e.g., for treatment with a L mimetic, an R mimetic or a K mimetic, or a combination thereof.

The disclosure contemplates methods of treating or preventing cartilage hair hypoplasia. In an embodiment, a method of treating or preventing cartilage hair hypoplasia in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of treating or preventing cartilage hair hypoplasia in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of treating or preventing cartilage hair hypoplasia in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of treating or preventing cartilage hair hypoplasia in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of treating or preventing cartilage hair hypoplasia in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of treating or preventing cartilage hair hypoplasia in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

Cartilage hair hypoplasia involves a genetic defect in the RMRP gene and is marked by hypoplastic anemia, short limbed dwarfism, and hypoplastic hair. A subject is diagnosed with cartilage hair hypoplasia via RMRP sequencing. In some embodiments, the method includes diagnosing a subject with cartilage hair hypoplasia. In some embodiments, the method further includes selecting a subject who has been diagnosed with cartilage hair hypoplasia, e.g., for treatment with a L mimetic, an R mimetic or a K mimetic, or a combination thereof.

The disclosure contemplates methods of treating or preventing Treacher Collins syndrome. In an embodiment, a method of treating or preventing Treacher Collins syndrome in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of treating or preventing Treacher Collins syndrome in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of treating or preventing Treacher Collins syndrome in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of treating or preventing Treacher Collins syndrome in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of treating or preventing Treacher Collins syndrome in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of treating or preventing Treacher Collins syndrome in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

Treacher Collins syndrome involves a genetic defect in the TCOF1 gene and is marked craniofacial abnormalities. A subject is diagnosed with Treacher Collins syndrome via a physical exam, or imaging, for example for craniofacial changes due to symmetrically or bilaterally decreased growth of structures of the first and second pharyngeal arch, groove, or pouch. A subject presenting with Treacher Collins syndrome may display one or more complications of craniofacial dystosis, for example complications related to airway, swallowing, brain development, and haring. In some embodiments, the method includes diagnosing a subject with Treacher Collins syndrome. In some embodiments, the method further includes selecting a subject who has been diagnosed with Treacher Collins syndrome, e.g., for treatment with a L mimetic, an R mimetic or a K mimetic, or a combination thereof.

The disclosure contemplates methods of treating or preventing a cohesinopathy, such as Roberts syndrome or Cornelia de Lange Syndrome.

In an embodiment, a method of treating or preventing Roberts syndrome in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of treating or preventing Roberts syndrome in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of treating or preventing Roberts syndrome in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of treating or preventing Roberts syndrome in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of treating or preventing Roberts syndrome in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of treating or preventing Roberts syndrome in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

In an embodiment, a method of treating or preventing Cornelia de Lange syndrome in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of treating or preventing Cornelia de Lange syndrome in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of treating or preventing Cornelia de Lange syndrome in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of treating or preventing Cornelia de Lange syndrome in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of treating or preventing Cornelia de Lange syndrome in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of treating or preventing Cornelia de Lange syndrome in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

In another embodiment, the invention provides a method of promoting re-myelination of or preventing myelin loss in nerve cells in a subject. mTORC1 has been identified as a crucial regulator of CNS myelination and myelin maintenance in oligodendrocytes and inhibition of mTORC1 activity has been associated with hypomyelination (Wahl, S E et al, Mammalian Target of Rapamycin Promotes Oligodendrocyte Differentiation, Initiation and Extent of CNS Myelination. J Neurosci 34, 4453-65 (2014)). mTORC1 has also been shown to have important roles in peripheral nervous system (PNS) myelination (see, e.g., Norrmén C et al., mTORC1 Controls PNS Myelination along the mTORC1-RXRγ-SREBP-Lipid Biosynthesis Axis in Schwann Cells, Cell Rep. 2014 Oct. 7. pii: S2211-1247(14)00769-4. doi: 10.1016/j.celrep.2014.09.001). Promoting re-myelination, myelin maintenance and/or repair, and/or preventing myelin loss or break-down (e.g., age-related) can be used to treat diseases, conditions, and disorders associated with demyelination, and/or hypomyelination (CNS and/or PNS). Examples of diseases, conditions, and disorders associated with demyelination, and/or hypomyelination include optic neuritis (in subjects having and not having MS), transverse myelitis, chronic inflammatory demyelinating polyneuropathy (CIDP) (also known as chronic relapsing polyneuropathy (CRP) or chronic inflammatory demyelinating polyradiculoneuropathy), Guillain-Barré syndrome (GBS), demyelination associated with vitamin B12 deficiency or malabsorption (e.g., subacute combined degeneration of spinal cord) and central pontine myelinolysis, which presents most commonly as a complication of treatment of patients with chronic severe hyponatremia (low sodium) but can also occur in patients with a history of chronic alcoholism or other conditions related to decreased liver function, and hypomyelinating leukodystrophies, such as 18q-syndrome, Cockayne syndrome, hypomyelination with atrophy of the basal ganglia and cerebellum, hypomyelation with congenital cataracts, hypomyelination of early myelinated structures, hypomyelination with brainstem and spinal cord involvement and leg spasticity, free sialic acid storage disease, fucosidosis, Pelizaeus-Merzbacher disease and Pelizaeus-Merzbacher-like disease, Pol III-related leukodystrophies/4H, Oculodentodigital dysplasia, RARS-associated hypomyelination, SOX 10-associated disorders, and trichothiodystrophy with hypersensitivity to sunlight (for additional information on hypomyelinating leukodystrophies see Pouwels, et al., “Hypomyelinating Leukodystrophies: Translational Research Progress and Propsects, Ann Neurol; 76:5-19 (2014)).

In one aspect of this embodiment, the method is used to treat a neurodegenerative disease characterized by loss of myelination of nerve cells. In a more specific aspect, the method is used to treat multiple sclerosis. In some embodiments, the subject is a subject diagnosed with or suspected of having a disease, disorder, or condition associated with demyelination, decreased myelin repair, defective myelin formation, etc. In some embodiments the method includes a step of diagnosing the subject with the disease, disorder, and/or condition. In some embodiments the method includes a step of selecting a subject that has been treated for the disease, disorder, and/or condition.

In an embodiment, a method of promoting re-myelination of nerve cells in a subject in need thereof comprises administering to the subject an effective amount of a leucine mimetic, an arginine mimetic, and a lysine mimetic, or a composition comprising the leucine mimetic, an arginine mimetic or a lysine mimetic. In some embodiments, a method of a method of promoting re-myelination of nerve cells in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic. In some embodiments, a method of a method of promoting re-myelination of nerve cells in a subject in need thereof comprises administering to the subject an effective amount of a leucine mimetic, an arginine mimetic, and a lysine mimetic, or a composition comprising the leucine mimetic, an arginine mimetic and the lysine mimetic.

In an embodiment, a method of a method of promoting re-myelination of nerve cells in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky. In an embodiment, a method of a method of promoting re-myelination of nerve cells in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl. In an embodiment, a method of a method of promoting re-myelination of nerve cells in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl. It should be appreciated that the leucine carboxy alkyl, arginine carboxy alkyl, and lysine carboxy alkyl can be any carboxy alkyl described herein (e.g., carboxy methyl ester, carboxy ethyl ester, etc.).

In certain aspects, the method of increasing mTORC1 activity/activation disclosed herein is used for preventing autophagy. Preventing autophagy can be useful, for example, as part of a therapeutic strategy for targeting certain cancers. (Chen & White. Cancer Prev Res; 4(7); 973-83 (2011). The data found in Example 4 below establishes a link between mTORC1 activation and the prevention of autophagy. Accordingly, it is believed that agents which activate mTORC1, such as the L, R, or K mimetics disclosed herein, can be used to prevent autophagy.

Generally, a method for preventing autophagy involves administering a L, R, or K mimetic as described herein or a composition comprising the same to a subject in need thereof. In some embodiments, a method of preventing autophagy in a subject in need thereof comprises administering to the subject an effective amount of a leucine mimetic, an arginine mimetic, or a lysine mimetic, or a composition comprising the leucine mimetic, an arginine mimetic or a lysine mimetic.

In some embodiments, a method of preventing autophagy in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic.

In some embodiments, a method of preventing autophagy in a subject in need thereof comprises administering to the subject an effective amount of a leucine mimetic, an arginine mimetic, and a lysine mimetic, or a composition comprising the leucine mimetic, an arginine mimetic and the lysine mimetic.

In some embodiments, a method of preventing autophagy in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky.

In some embodiments, a method of preventing autophagy in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl.

In some embodiments, a method of preventing autophagy in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl.

Those skilled in the art will appreciate that the methods of preventing autophagy can be performed in any subject which is in need of such treatment. In some embodiments, the subject is a subject diagnosed with or suspected of having a disease, disorder, or condition associated with increased or dysregulated autophagy. In some embodiments, the subject is a subject that has been diagnosed with cancer. In some embodiments, the cancer is a cancer associated with expression of an oncogenic Ras gene. In some embodiments, the subject is a subject at risk of developing a cancer associated with expression of an oncogenic Ras gene. In some embodiments, the subject has at least one mutation in the Ras gene.

In certain aspects, the methods of increasing mTORC1 activity/activation disclosed herein are used for inducing satiety or reducing feeding in a subject in need thereof. The data found in Example 4 is consistent with the hypothesis that activation of mTORC1 induces satiety. Accordingly, it is believed that agents which activate mTORC1, such as the L, R, or K mimetics disclosed herein, can be used to induce satiety or otherwise reduce feeding.

Generally, a method for inducing satiety involves administering a L, R, or K mimetic or a composition comprising the same to a subject in need thereof.

In some embodiments, a method of inducing satiety in a subject in need thereof comprises administering to the subject an effective amount of a leucine mimetic, an arginine mimetic, or a lysine mimetic, or a composition comprising the leucine mimetic, an arginine mimetic or a lysine mimetic.

In some embodiments, a method of inducing satiety in a subject in need thereof comprises administering to the subject an effective amount of at least two of a leucine mimetic, an arginine mimetic, and a lysine mimetic, or a composition comprising the at least two of the leucine mimetic, an arginine mimetic, and the lysine mimetic.

In some embodiments, a method of inducing satiety in a subject in need thereof comprises administering to the subject an effective amount of a leucine mimetic, an arginine mimetic, and a lysine mimetic, or a composition comprising the leucine mimetic, an arginine mimetic and the lysine mimetic.

In some embodiments, a method of inducing satiety in a subject in need thereof comprises administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky.

In some embodiments, a method of inducing satiety in a subject in need thereof comprises administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl.

In some embodiments, a method of inducing satiety in a subject in need thereof comprises administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl.

The disclosure contemplates inducing satiety or otherwise reducing feeding in any subject in which inducing satiety or minimizing calorie intake is desirable. In some embodiments, the subject has, is at risk of developing, or is suspected of having obesity. In some embodiments, the subject has, is at risk of developing, or is suspected of having diabetes. In some embodiments, the subject has, is at risk of developing, or is suspected of having metabolic syndrome. In some embodiments, the subject has a body mass index of at least 25. In some embodiments, the subject has a body mass index of at least 30. In certain aspects, for the method of increasing mTORC1 activity/activation disclosed herein are used for treating a disease, condition, or disorder which would benefit by activating mTORC1 in a subject. The present disclosure contemplates treating any disease, condition, or disorder which would benefit by activating mTORC1 in a subject, for example, those in which mTORC1 activity is decreased or suppressed, or in which an increase in mTORC1 activity or activation induces a relevant physiological effect. Recent literature indicates that mTORC1 activation may induce sustained antidepressant-like effects (see, e.g., Chaki et al., “Involvement of the mammalian target of rapamycin signaling in the antidepressant-like effect of group II metabotropic glutamate receptor antagonists,” Neuropharmacology 61:1419-1423 (2011), and Aghajanian et al., “Signaling Pathways Underlying the Rapid Antidepressant Actions of Ketamine,” Neuropharmacology 62(1): 35-41 (2012)). Accordingly, it is believed that agents which activate mTORC1, such as the L, R, or K mimetics disclosed herein, can be used to treat diseases, conditions, or disorders which would benefit by activating mTORC1, such as depressive disorders and condition associated therewith. Other diseases, conditions and disorders that would benefit from activating mTORC1 include jet lag, preventing or reversing cardiac muscle atrophy (e.g., where a subject is suffering from or has suffered from heart attack, congestive heart failure, heart transplant, heart valve repair, atherosclerosis, other major blood vessel disease, and heart bypass surgery).

Generally, a method for treating a disease, condition, or disorder which would benefit by activating mTORC1 in a subject involves administering a L, R, or K mimetic as described herein or a composition comprising the same to a subject in need thereof.

In some embodiments, a method of treating a disease, condition, or disorder which would benefit by activating mTORC1 in a subject, the method comprising administering to the subject an effective amount of a leucine mimetic, an arginine mimetic, or a lysine mimetic, or a composition comprising the leucine mimetic, an arginine mimetic or a lysine mimetic, thereby activating mTORC1 in the subject.

In some embodiments, a method of treating a disease, condition, or disorder which would benefit by activating mTORC1 in a subject, the method comprising administering to the subject an effective amount of at least two of a leucine mimetic, an arginine mimetic, and a lysine mimetic, or a composition comprising the at least two of the leucine mimetic, an arginine mimetic, and the lysine mimetic, thereby activating mTORC1 in the subject.

In some embodiments, a method of treating a disease, condition, or disorder which would benefit by activating mTORC1 in a subject, the method comprising administering to the subject an effective amount of a leucine mimetic, an arginine mimetic, and a lysine mimetic, or a composition comprising the leucine mimetic, an arginine mimetic and the lysine mimetic, thereby activating mTORC1 in the subject.

In some embodiments, a method of treating a disease, condition, or disorder which would benefit by activating mTORC1 in a subject, the method comprising administering to the subject an effective amount of a leucine carboxy alkyl, an arginine carboxy alkyl, or a lysine carboxy alkyl, or a composition comprising the leucine carboxy alkyl, the arginine carboxy alkyl, or the lysine carboxy alky, thereby activating mTORC1 in the subject.

In one embodiment, a method of treating a disease, condition, or disorder which would benefit by activating mTORC1 in a subject, the method comprising administering to the subject an effective amount of at least two of a leucine carboxy alkyl, an arginine carboxy alkyl, or a lysine carboxy alkyl, or a composition comprising the at least two of the leucine carboxy alkyl, an arginine carboxy alkyl, and the lysine carboxy alkyl, thereby activating mTORC1 in the subject.

In some embodiments, a method of treating a disease, condition, or disorder which would benefit by activating mTORC1 in a subject, the method comprising administering to the subject an effective amount of a leucine carboxy alkyl, an arginine carboxy alkyl, and a lysine carboxy alkyl, or a composition comprising the leucine carboxy alkyl, the arginine carboxy alkyl, and the lysine carboxy alkyl, thereby activating mTORC1 in the subject.

Those skilled in the art will appreciate that the methods of treating a disease, condition, or disorder which would benefit by activating mTORC1 in a subject can be performed in any subject which is in need of such treatment.

In some embodiments, the subject is a subject diagnosed with or suspected of having a depressive disorder. In some embodiments, the subject is a subject suffering from, diagnosed with or suspected of having, or experiencing symptoms associated with depression. In some embodiments, the subject is a subject suffering from, diagnosed with or suspected of having, or experiencing symptoms associated with anxiety. In some embodiments, the subject is a subject suffering from, diagnosed with or suspected of having or experiencing symptoms associated with chronic stress. In some embodiments, the subject is a subject suffering from, diagnosed with or suspected of having or experiencing symptoms associated with a disorder due to decreased synaptogenesis. In some embodiments, the subject is a subject suffering from, diagnosed with or suspected of having or experiencing symptoms associated with a disorder involving neuronal atrophy. In some embodiments, the subject is a subject suffering from, diagnosed with or suspected of having or experiencing symptoms associated with a long-term memory deficiency. In some embodiments, the subject is a subject suffering from, diagnosed with or suspected of having or experiencing symptoms associated with a learning deficiency.

As used herein, “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of, for example, atrophy, delay or slowing of atrophy, and an increased lifespan as compared to that expected in the absence of treatment.

As used herein, the term “administering,” refers to the placement of the L mimetic, R mimetic, or K mimetic, or a composition comprising the leucine mimetic, the arginine mimetic, and the lysine mimetic as disclosed herein into a subject by a method or route which results in delivery to a site of action. The pharmaceutical composition comprising the leucine mimetic, the arginine mimetic, and the lysine mimetic can be administered by any appropriate route which results in an effective treatment in the subject.

A therapeutically effective amount is an amount of an agent that is sufficient to produce a statistically significant, measurable change in, for example, muscle atrophy (e.g., improving muscle recovery after immobilization-induced atrophy). Such effective amounts can be gauged in clinical trials as well as animal studies. Efficacy of an agent can be determined by assessing physical indicators of, for example muscle atrophy (e.g., increased muscle mass or strength). In experimental systems, assays for efficacy include measurement of skeletal muscle mass, for example. Such assays are well known in the art. The efficacy of a given treatment for muscle atrophy or a disorder, condition, or symptom associated with muscle atrophy can be determined by the skilled clinician. However, a treatment is considered “effective treatment,” as the term is used herein, if any one or all of the signs or symptoms of e.g., a muscle atrophy are altered in a beneficial manner, other clinically accepted symptoms are improved or ameliorated, e.g., by at least 10% following treatment with an agent or composition as described herein. Efficacy can also be measured by a failure of an individual to worsen as assessed by, e.g., hospitalization or need for medical interventions (i.e., progression of the disease is halted), or by any suitable method of assessment (e.g., physical examination). Methods of measuring these indicators are known to those of skill in the art and/or described herein.

In some embodiments, the methods further comprise administering the compositions (e.g., pharmaceutical compositions) described herein along with one or more additional agents, biologics, drugs, or treatments beneficial to a subject suffering from muscle atrophy as part of a combinatorial therapy. In some such embodiments, the agent, biologic, drug, or treatment can be selected from the group consisting of: modulators of one or more of skeletal myosin, skeletal actin, skeletal tropomyosin, skeletal troponin C, skeletal troponin I, skeletal troponin T, and skeletal muscle, including fragments and isoforms thereof, and the skeletal sarcomere and other suitable therapeutic agents useful in the treatment of the aforementioned diseases including: anti-obesity agents, anti-sarcopenia agents, anti-wasting syndrome agents, anti-frailty agents, anti-cachexia agents, anti-muscle spasm agents, agents against post-surgical and post-traumatic muscle weakness, and anti-neuromuscular disease agents, as well as the agents described in U.S. Patent Application No. 2005/0197367.

Suitable additional medicinal and pharmaceutical agents include, for example: orlistat, sibutramine, diethylpropion, phentermine, benzphetamine, phendimetrazine, estrogen, estradiol, levonorgestrel, norethindrone acetate, estradiol valerate, ethinyl estradiol, norgestimate, conjugated estrogens, esterified estrogens, medroxyprogesterone acetate, insulin-derived growth factor, human growth hormone, riluzole, cannabidiol, prednisone, beta agonists (e.g., albuterol), myostatin inhibitors, selective androgen receptor modulators, non-steroidal anti-inflammatory drugs, and botulinum toxin.

Other suitable medicinal and pharmaceutical agents include TRH, diethylstilbestrol, theophylline, enkephalins, E series prostaglandins, compounds disclosed in U.S. Pat. No. 3,239,345 (e.g., zeranol), compounds disclosed in U.S. Pat. No. 4,036,979 (e.g., sulbenox), peptides disclosed in U.S. Pat. No. 4,411,890 growth hormone secretagogues such as GHRP-6, GHRP-1 (disclosed in U.S. Pat. No. 4,411,890 and publications WO 89/07110 and WO 89/07111), GHRP-2 (disclosed in WO 93/04081), NN703 (Novo Nordisk), LY444711 Lilly), MK-677 (Merck), CP424391 (Pfizer) and B-HT920, growth hormone releasing factor and its analogs, growth hormone and its analogs and somatomedins including IGF-1 and IGF-2, leukemia inhibitory factor, cilia neurotrophic factor, brain derived neurotrophic factor, interleukin 6, interleukin 15, alpha-adrenergic agonists, such as clonidine or serotonin 5-HT_{D} agonists, such as sumatriptan, agents which inhibit somatostatin or its release, such as physostigmine, pyridostigmine, parathyroid hormone, PTH(1-34), and bisphosphonates, such as MK-217 (alendronate).

Still other suitable medicinal and pharmaceutical agents include estrogen, testosterone, selective estrogen receptor modulators, such as tamoxifen or raloxifene, other androgen receptor modulators, such as those disclosed in Edwards, J. P. et. al., Bio. Med. Chem. Let., 9, 1003-1008 (1999) and Hamann, L. G. et. al., J. Med. Chem., 42, 210-212 (1999), and progesterone receptor agonists (“PRA”), such as levonorgestrel, medroxyprogesterone acetate (MPA).

Still other suitable medicinal and pharmaceutical agents include aP2 inhibitors, such as those disclosed in U.S. Ser. No. 09/519,079 filed Mar. 6, 2000, PPAR gamma antagonists, PPAR delta agonists, beta 2 adrenergic agonists, beta 3 adrenergic agonists, such as AJ9677 (Takeda/Dainippon), L750355 (Merck), or CP331648 (Pfizer), other beta 3 agonists as disclosed in U.S. Pat. Nos. 5,541,204, 5,770,615, 5,491,134, 5,776,983 and 5,488,064, a lipase inhibitor, such as orlistat or ATL-962 (Alizyme), a serotonin (and dopamine) reuptake inhibitor, such as sibutramine, topiramate (Johnson & Johnson) or axokine (Regeneron), a thyroid receptor beta drug, such as a thyroid receptor ligand as disclosed in WO 97/21993, WO 99/00353, and GB98/284425, and anorectic agents, such as dexamphetamine, phentermine, phenylpropanolamine or mazindol.

Still other suitable medicinal and pharmaceutical agents include HIV and AIDS therapies, such as indinavir sulfate, saquinavir, saquinavir mesylate, ritonavir, lamivudine, zidovudine, lamivudine/zidovudine combinations, zalcitabine, didanosine, stavudine, and megestrol acetate.

Still other suitable medicinal and pharmaceutical agents include antiresorptive agents, hormone replacement therapies, vitamin D analogues, elemental calcium and calcium supplements, cathepsin K inhibitors, MMP inhibitors, vitronectin receptor antagonists, Src SH₂ antagonists, vascular—H⁺ ATPase inhibitors, ipriflavone, fluoride, Tibolone, prostanoids, 17-beta hydroxysteroid dehydrogenase inhibitors and Src kinase inhibitors.

The above other therapeutic agents, when employed in combination with the chemical entities described herein, may be used, for example, in those amounts indicated in the Physicians' Desk Reference (PDR) or as otherwise determined by one of ordinary skill in the art.

In some embodiments, the methods further comprise selecting a subject that has been diagnosed with a disorder, condition, or symptom associated with muscle atrophy. As used herein, “muscle atrophy” refers to a decrease in the skeletal muscle mass. In some embodiments, a subject that has been diagnosed with a disorder, condition, or symptom associated with muscle atrophy is a subject whose skeletal muscle mass has decreased by at least a 5% as a result of the disorder, condition, or symptom. In some embodiments, a subject that has been diagnosed with a disorder, condition, or symptom associated with muscle atrophy is a subject whose skeletal muscle mass has decreased by at least a 5%, 8%, 10%, 11%, 12%, 13%, 14%, 15%, 16%, 17%, 18%, 19%, 20%, 21%, 22%, 23%, 24%, 25%, 26%, 27%, 28%, 29%, 30%, or more as a result of the disorder, condition, or symptom. In some embodiments, a subject that has been diagnosed with a disorder, condition, or symptom associated with muscle atrophy is a subject whose skeletal muscle mass has decreased by at least a 25%, 28%, 30%, 31%, 32%, 33%, 34%, 35%, 36%, 37%, 38%, 39%, 40%, 41%, 42%, 43%, 44%, 45%, 46%, 47%, 48%, 49%, 50%, or more as a result of the disorder, condition, or symptom. In some embodiments, a subject that has been diagnosed with a disorder, condition, or symptom associated with muscle atrophy is a subject whose muscle weight relative to body weight ratio decreased by at least a 2%, at least a 3%, at least a 4%, at least a 5%, at least 6%, at least 7%, at least 8%, at least 9%, at least 10%, at least a 15%, at least a 16%, at least a 20%, at least a 25%, at least 30%, at least 35%, or at least 40% or more as a result of the disorder, condition, or symptom.

In some embodiments, the methods further comprise selecting a subject that has been diagnosed with a disorder, condition, or symptom associated with muscle atrophy due to aging. As used herein, “muscle atrophy due to aging” refers to muscle atrophy which is attributable to a subject's age. In some embodiments, the subject is one who is at risk of developing a disorder, condition, or symptom associated with muscle atrophy due to aging. In some embodiments, the subject is an elderly subject. In some embodiments, an elderly subject is over the age of 50, 55, 60, 65, 70, 75, 80, 85, 90, or 100 years of age.

In some embodiments, the methods further comprise selecting a subject that has been diagnosed with a disorder, condition, or symptom associated with muscle atrophy due to unloading stress. In some embodiments, the methods further comprise selecting a subject that has been diagnosed with a disorder, condition, or symptom associated with muscle atrophy due to cancer. In some embodiments, the methods further comprise selecting a subject that has been diagnosed with a disorder, condition, or symptom associated with muscle atrophy due to an autoimmune disorder. In some embodiments, the methods further comprise selecting a subject that has been diagnosed with a disorder, condition, or symptom associated with muscle atrophy due to HIV. In some embodiments, the methods further comprise selecting a subject that has been diagnosed with a disorder, condition, or symptom associated with muscle atrophy due to dystrophy. In some embodiments, the methods further comprise selecting a subject that has been diagnosed with a disorder, condition, or symptom associated with muscle atrophy due to malnutrition or starvation. In some embodiments, the methods further comprise selecting a subject that has been diagnosed with a disorder, condition, or symptom associated with muscle atrophy due to immobilization. In some embodiments, the immobilization is due to sports injury. In some embodiments, the immobilization is due to rehabilitation. In some embodiments, the immobilization is due to hospitalization. In some embodiments, the immobilization is due to casting. In some embodiments, the immobilization is due to bed rest. In some embodiments, the immobilization is limb immobilization. In some embodiments, the methods further comprise selecting a subject that has been diagnosed with a disorder, condition, or symptom associated with muscle atrophy due to decreased mobility (e.g., joint mobility).

In some embodiments, citrulline can be used in place of an arginine carboxy alkyl in the treatment of any of the diseases or conditions set forth in this “Method of Treatment” section.

In some embodiments, treatment of any of the diseases or conditions set forth in this “Method of Treatment” section comprises administration to a subject in need thereof of a combination of leucine, citrulline and, optionally, lysine. Each of the leucine, citrulline and, optionally, lysine may be administered in separate dosage forms or combined with one or more of the other two components in a single dosage form. In one aspect of these embodiments, lysine is administered at less than 50%, less than 40%, less than 30%, less than 20% or 10% of the molar equivalent of leucine.

In some embodiments, treatment of any of the diseases or conditions set forth in this “Method of Treatment” section comprises administration to a subject in need thereof of a combination of leucine ethyl ester, citrulline and, optionally, lysine. Each of the leucine ethyl ester, citrulline and, optionally, lysine may be administered in separate dosage forms or combined with one or more of the other two components in a single dosage form. In one aspect of these embodiments, lysine is administered at less than 50%, less than 40%, less than 30%, less than 20% or 10% of the molar equivalent of leucine being administered. In another aspect of these embodiments, the leucine ethyl ester is administered at less than 90%, less than 80%, less than 70%, less than 60%, or 50% of the molar equivalent of citrulline being administered.

In some embodiments, treatment of any of the diseases or conditions set forth in this “Method of Treatment” section comprises administration to a subject in need thereof of a Leu-Arg-Lys tripeptide.

In some embodiments, treatment of any of the diseases or conditions set forth in this “Method of Treatment” section comprises administration to a subject in need thereof of a Leu-Citrulline-Lys tripeptide.

In some embodiments, the disease or condition to be treated is selected from those resulting in skeletal muscle atrophy (such as sarcopenia, muscle denervation, prolonged immobilization and muscular dystrophy), decreased satiety (such as obesity and other conditions characterized by overeating, or hyperphagia), ribosomopathies (e.g. Diamond-Blackfan anemia, 5q-syndrome, Shwachman-Diamond syndrome, X-linked dyskeratosis, cartilage hair hypoplasia, and Treacher Collins syndrome), cohesinopathies (e.g. Roberts syndrome and Cornelia de Lange syndrome) and conditions characterized by demyelination of nerves (e.g., multiple sclerosis).

### Compositions

The disclosure contemplates various compositions comprising a L mimetic, an R mimetic, or a K mimetic described herein. In an aspect, the disclosure provides a composition comprising an effective amount of at least one amino acid mimetic selected from the group consisting of a leucine mimetic, an arginine mimetic, and a lysine mimetic. In an aspect, the disclosure provides a composition comprising effective amounts of at least two amino acid mimetics selected from the group consisting of a leucine mimetic, an arginine mimetic, and a lysine mimetic. In an aspect, the disclosure provides a composition comprising effective amounts of a leucine mimetic, an arginine mimetic, and a lysine mimetic.

In some embodiments, the composition does not include a L mimetic consisting of the native amino acid L. In some embodiments, the composition does not include an R mimetic consisting of the native amino acid R. In some embodiments, the composition does not include a K mimetic consisting of the native amino acid K. In some embodiments, at least one of the leucine mimetic, the arginine mimetic, and the lysine mimetic comprises a non-native form of the respective amino acids leucine, arginine, and lysine.

In some embodiments, the composition does not include a non-essential amino acid. In some embodiments, the composition includes no more than 1%, no more than 2%, no more than 3%, no more than 4%, no more than 5%, no more than 6%, no more than 7%, no more than 8%, no more than 9%, or no more than up to 10% of non-essential amino acids.

In some embodiments, the composition does not include essential amino acids other than leucine, arginine, and lysine. In some embodiments, the composition includes no more than 1%, no more than 2%, no more than 3%, no more than 4%, no more than 5%, no more than 6%, no more than 7%, no more than 8%, no more than 9%, or no more than up to 10% of essential amino acids other than leucine, arginine, and lysine.

In some embodiments, the composition includes at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 25%, at least 30%, at least 35%, at least 40%, at least 45%, at least 46%, at least 47%, at least 48%, at least 49%, or at least 50% or more of a derivative, analog, metabolite, or metabolic byproduct of the native amino acids leucine, arginine and/or lysine.

In some embodiments, the composition does not include whey protein isolate. In some embodiments, the composition does not include soy protein isolate. In some embodiments, the composition does not include casein. In some embodiments, the composition does not include caseinate. In some embodiments, the composition does not include a dietary source of at least one of leucine, arginine, and/or lysine.

In some embodiments, at least one of the L, R, and K mimetics comprises an unnaturally occurring source of L, R, and K. In some embodiments, the L, R, and K mimetics comprise carboxy alkyl derivatives of L, R, and K.

In an aspect, the disclosure provides a composition comprising effective amounts of L, a metabolite of L, or a source of L, R, a metabolite of R, or a source of R, and K, a metabolite of K or a source of K.

In some embodiments, the L, R, and K are isolated and/or purified. In some embodiments, the sources of L, R, and K are not naturally occurring sources. In some embodiments, the sources of L, R, and K are isolated and/or purified sources of L, R, and K.

In some embodiments, all of the L or the source of L, R or the source of R, and K or the source of K comprises carboxy alkyl derivatives of L or the source of L, R or the source of R, and K or the source of K. In some embodiments, all of the L or the source of L, R or the source of R, and K or the source of K comprises carboxy ester derivatives of L or the source of L, R or the source of R, and K or the source of K.

In some embodiments, at least two of the L or the source of L, R or the source of R, and K or the source of K comprises carboxy ester derivatives (e.g., methyl esters and ethyl esters).

In some embodiments, the source of R is citrulline.

In some embodiments, a composition comprises a leucine carboxy ester, arginine, and a lysine carboxy ester. In some embodiments, a composition comprises a leucine carboxy ester, arginine, and a lysine carboxy ester. In some embodiments, a composition comprises leucine methyl ester, arginine, and lysine methyl ester. In some embodiments, a composition comprises leucine ethyl ester, arginine, and lysine methyl ester. In some embodiments, a composition comprises leucine methyl ester, arginine, and lysine ethyl ester. In some embodiments, a composition comprises leucine ethyl ester, arginine, and lysine ethyl ester.

In some embodiments, a composition comprises a leucine carboxy ester, citrulline, and a lysine carboxy ester. In some embodiments, a composition comprises a leucine carboxy ester, citrulline, and a lysine carboxy ester. In some embodiments, a composition comprises leucine methyl ester, citrulline, and lysine methyl ester. In some embodiments, a composition comprises leucine ethyl ester, citrulline, and lysine methyl ester. In some embodiments, a composition comprises leucine methyl ester, citrulline, and lysine ethyl ester. In some embodiments, a composition comprises leucine ethyl ester, citrulline, and lysine ethyl ester.

In an aspect, the disclosure provides a composition comprising at least two carboxy ester derivatives and one native amino acid selected from the group consisting of leucine (L), arginine (R) and lysine (K). In some embodiments, the native amino acid selected from the group consisting of L, R, and K is isolated and/or purified. In some embodiments, the at least two carboxy ester derivative amino acids are carboxy ester derivatives of leucine and lysine. In some embodiments, the native amino acid is arginine. In some embodiments, the carboxy ester derivatives of leucine and lysine are selected from the group consisting of methyl esters, and ethyl esters.

In an aspect, the disclosure provides a composition comprising leucine alkyl ester and arginine alkyl ester.

In an aspect, the disclosure provides a composition comprising leucine ethyl ester and arginine ethyl ester.

In a second particular embodiment, the disclosure provides a composition for administration to a subject consisting essentially of:

a. a first component selected from L-arginine; an mTORC1 agonizing arginine mimetic; and a peptide, non-standard peptide, polypeptide, non-standard polypeptide, protein or non-standard protein any of which is enriched for one or both of L-arginine residues or mTORC1 agonizing arginine mimetic residues; and

b. a second component selected from L-leucine; an mTORC1 agonizing leucine mimetic; and a peptide, non-standard peptide, polypeptide, non-standard polypeptide, protein or non-standard protein any of which is enriched for one or both of L-leucine residues or mTORC1 agonizing leucine mimetic residues;

c. optionally, a third component selected from L-lysine; an mTORC1 agonizing lysine mimetic; and a peptide, non-standard peptide, polypeptide, non-standard polypeptide, protein or non-standard protein any of which is enriched for one or both of L-lysine residues or mTORC1 agonizing lysine mimetic residues; and

d. optionally, one or more excipients.

In some aspects of the second particular embodiment, the third component is administered to the subject.

In some aspects of the second particular embodiment, at least one of the first component, second component or optional third component is other than a naturally occurring L-form of an amino acid.

In some aspects of the second particular embodiment, the first component is L-arginine or an mTORC1 agonizing arginine mimetic selected from a carboxy terminal modified form of L-arginine and a side-chain modified form of L-arginine. More specifically, the first component is selected from L-arginine, a L-arginine ester,

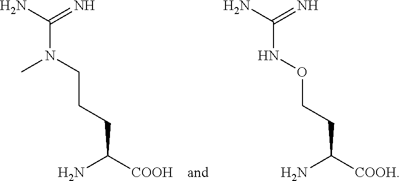

Even more specifically, the L-arginine ester is L-arginine ethyl ester. In an alternate specific aspect, the first component is citrulline.

In some aspects of the second particular embodiment, the second component is L-leucine or an mTORC1 agonizing leucine mimetic selected from a carboxy terminal modified form of L-leucine, an amino terminal modified form of L-leucine, a side-chain modified form of L-leucine, and L-methionine. In a more specific aspect, the second component is selected from L-leucine, a L-leucine ester, L-methionine

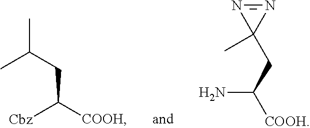

Even more specifically, the L-leucine ester is L-leucine ethyl ester.

In some aspects of the second particular embodiment, the third component, if present, is selected from L-lysine and an L-lysine ester. In some embodiments, the L-lysine ester is an L-lysine ethyl ester.

In yet other aspects of the second particular embodiment, at least one component is selected from:

a. a peptide, non-standard peptide, polypeptide, non-standard polypeptide, protein or non-standard protein any of which is enriched for one or both of L-arginine residues or mTORC1 agonizing arginine mimetic residues;

b. a peptide, non-standard peptide, polypeptide, non-standard polypeptide, protein or non-standard protein any of which is enriched for one or both of L-leucine residues or mTORC1 agonizing leucine mimetic residues; and

c. a peptide, non-standard peptide, polypeptide, non-standard polypeptide, protein or non-standard protein any of which is enriched for one or both of L-lysine residues or mTORC1 agonizing lysine mimetic residues; wherein:

any peptide, non-standard peptide, polypeptide or non-standard polypeptide is optionally associated with one or more of a half-life increasing moiety, a cell penetration moiety, a specific organ-directing moiety, and a specific cell-type-directing moiety.

In some aspects of the second particular embodiment, at least one component is selected from: a peptide, polypeptide, or protein any of which is enriched for L-arginine residues; a peptide, polypeptide, or protein any of which is enriched for L-leucine residues; and a peptide polypeptide, or protein any of which is enriched for L-lysine residues.

In some aspects of the second particular embodiment, at least two components are independently selected from:

a. a peptide, non-standard peptide, polypeptide, non-standard polypeptide, protein or non-standard protein any of which is enriched for one or both of L-arginine residues or mTORC1 agonizing arginine mimetic residues;

b. a peptide, non-standard peptide, polypeptide, non-standard polypeptide, protein or non-standard protein any of which is enriched for one or both of L-leucine residues or mTORC1 agonizing leucine mimetic residues; and

c. a peptide, non-standard peptide, polypeptide, non-standard polypeptide, protein or non-standard protein any of which is enriched for one or both of L-lysine residues or mTORC1 agonizing lysine mimetic residues.

In a more specific aspect each of the at least two component is independently selected from: a peptide, polypeptide, or protein any of which is enriched for L-arginine residues; a peptide, polypeptide, or protein any of which is enriched for L-leucine residues; and a peptide polypeptide, or protein any of which is enriched for L-lysine residues.

In another aspect of the second particular embodiment, the third component is present and each of the first, second and third components are independently selected from:

a. a peptide, non-standard peptide, polypeptide, non-standard polypeptide, protein or non-standard protein any of which is enriched for one or both of L-arginine residues or mTORC1 agonizing arginine mimetic residues;

b. a peptide, non-standard peptide, polypeptide, non-standard polypeptide, protein or non-standard protein any of which is enriched for one or both of L-leucine residues or mTORC1 agonizing leucine mimetic residues; and

c. a peptide, non-standard peptide, polypeptide, non-standard polypeptide, protein or non-standard protein any of which is enriched for one or both of L-lysine residues or mTORC1 agonizing lysine mimetic residues.

In a more specific aspect, each of the first, second and third components are independently selected from: a peptide, polypeptide, or protein any of which is enriched for L-arginine residues; a peptide, polypeptide, or protein any of which is enriched for L-leucine residues; and a peptide polypeptide, or protein any of which is enriched for L-lysine residues.

In another aspect of the second particular embodiment, at least two components are present on the same peptide, non-standard peptide, polypeptide, non-standard polypeptide, protein or non-standard protein which is enriched for at least two of:

a. L-arginine residues or mTORC1 agonizing arginine mimetic residues;

b. L-leucine residues or mTORC1 agonizing leucine mimetic residues; and

c. L-lysine residues or mTORC1 agonizing lysine mimetic residues.

In a more specific aspect, the at least two components are present on the same peptide, polypeptide, or protein which is enriched for at least two of: L-arginine residues; L-leucine residues; and L-lysine residues.

In still another aspect of the second particular embodiment, each of the first, second and third components is present on the same peptide, non-standard peptide, polypeptide, non-standard polypeptide, protein or non-standard protein which is enriched for

a. L-arginine residues or mTORC1 agonizing arginine mimetic residues;

b. L-leucine residues or mTORC1 agonizing leucine mimetic residues; and

c. L-lysine residues or mTORC1 agonizing lysine mimetic residues.

In a more specific aspect, each of the first, second and third component is present on the same peptide, polypeptide, or protein which is enriched for L-arginine residues, L-leucine residues, and L-lysine residues.

In another aspect of the second particular embodiment, each of the first, second and third component is present on the same peptide, non-standard peptide, polypeptide or non-standard polypeptide, wherein:

a. the peptide, non-standard peptide, polypeptide or non-standard polypeptide consists of residues selected from L-arginine residues, mTORC1 agonizing arginine mimetic residues, L-leucine residues or mTORC1 agonizing leucine mimetic residues, L-lysine residues and mTORC1 agonizing lysine mimetic residues; and

b. the peptide, non-standard peptide, polypeptide or non-standard polypeptide is optionally associated with one or more of a half-life increasing moiety, a cell penetration moiety, a specific organ-directing moiety, and a specific cell-type-directing moiety.

In a more specific aspect every component is present on the same peptide or polypeptide, and wherein the peptide or polypeptide consists of residues selected from L-arginine residues, L-leucine residues and, L-lysine residues; and, optionally, is associated with one or more of a half-life increasing moiety, a cell penetration moiety, a specific organ-directing moiety, and a specific cell-type-directing moiety. In another more specific aspect, every component is present on one peptide or polypeptide, and wherein the peptide or polypeptide comprises at least one mTORC1 agonizing arginine mimetic residue, mTORC1 agonizing leucine mimetic residue, or mTORC1 agonizing lysine mimetic residue; and, optionally is associated with one of more of a half-life increasing moiety, a cell penetration moiety, a specific organ-directing moiety, and a specific cell-type-directing moiety. In an even more specific aspect the at least one mTORC1 agonizing arginine mimetic residue, mTORC1 agonizing leucine mimetic residue, or mTORC1 agonizing lysine mimetic residue is selected from a L-arginine ester,

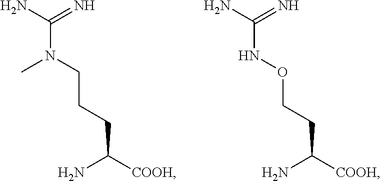

a L-leucine ester, L-methionine,

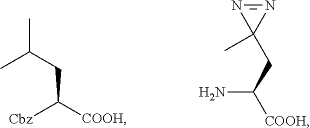

and corresponding monovalent and divalent radicals thereof. In yet another specific aspect the peptide, non-standard peptide, polypeptide or non-standard polypeptide is between two and thirty residues in length, more specifically between two and twelve residues in length.

In yet another aspect of the second particular embodiment, at least one peptide, non-standard peptide, polypeptide, non-standard polypeptide, protein or non-standard protein comprises a cell penetration amino acid sequence.

In still another specific aspect of the second particular embodiment, the peptide is the tripeptide Leu-Arg-Lys. In still another specific aspect of the second particular embodiment, the peptide is the tripeptide Leu-Citrulline-Lys.

In any of the above aspects of the second particular embodiment, the composition may be formulated into a pharmaceutically acceptable composition or a nutraceutical composition. Such composition may, for example, be designed for oral, parenteral, intra-muscular or direct to brain administration. In a more specific aspect, at least one of the components is formulated into a controlled release formulation. In another more specific aspect, at least one of the components in the composition is formulated into a composition to promote absorption from a specific portion of the digestive tract. In an even more specific aspect, each of the components is formulated into either a controlled release formulation and/or a composition to promote absorption from a specific portion of the digestive tract. In still another more specific aspect, at least one of the components is formulated into a pharmaceutical composition for delivery to a specific organ or cell type (e.g., brain, muscle, fibroblasts, bone, cartilage, liver, lung, breast, skin, bladder, kidney, heart, smooth muscle, adrenal, pituitary, pancreas, melanocytes, blood, adipose, and intestine). In another more specific aspect, the composition is formulated for oral or intravenous administration. It should be understood, that when multiple compositions are used to achieve administration of the required and/or optional components, administration of any one composition may be achieved by the same or different route than any other composition. In a particularly specific aspect, the administration of any composition comprising citrulline is achieved by oral administration. In another particularly specific aspect, the administration of any composition comprising citrulline is achieved by intravenous administration. Without being bound by theory, we believe that administration of citrulline (whether as the sole component of a composition, part of a combination of components in a composition, or part of a peptide, polypeptide or protein composition) has less deleterious effect on the liver of the recipient as compared to the administration of arginine in a corresponding form.

In any of the above aspects of the second particular embodiment, the composition may be formulated to increase in Cₘₐₓ in the subject as compared to the Cₘₐₓ of a composition consisting of the corresponding component and a pharmaceutically acceptable buffer.

In any of the above aspects of the second particular embodiment, the composition may be formulated to increase in Cₘᵢₙ in the subject as compared to the Cₘᵢₙ of a composition consisting of the corresponding component and a pharmaceutically acceptable buffer.

The compositions may be any kind of composition that is suitable for human and/or animal consumption. For example, the compositions may comprise food compositions, dietary supplements, nutritional compositions, nutraceuticals, powdered nutritional products to be reconstituted in water or milk before consumption, food additives (e.g., added to feed of a non-human animal or to human food), medicaments, drinks, and pet food. In some embodiments, the composition does not comprise milk or milk proteins. In some embodiments, the food compositions do not comprise natural foods. In some embodiments, the food compositions comprise synthetic foods. In some embodiments, the food compositions comprise processed foods.

In some embodiments, the composition is a pharmaceutical composition. As used herein, the term “pharmaceutical composition” refers to the active agent in combination with a pharmaceutically acceptable carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The preparation of a pharmacological composition that contains active ingredients dissolved or dispersed therein is well understood in the art and generally need not be limited based on formulation.

The compositions described herein can be used for can be used in any of the methods described herein, e.g., for increasing skeletal muscle mass, promoting skeletal muscle anabolism, promoting skeletal muscle recovery (e.g., after immobilization-induced muscle atrophy), or for treating or preventing muscle atrophy or a disorder, condition, or symptom associated with characterized by muscle atrophy. In some embodiments, the disorder, condition, or symptom associated with or characterized by muscle atrophy is selected from the group consisting of aging, bony fractures, weakness, cachexia, denervation, diabetes, dystrophy, exercise-induced skeletal muscle fatigue, fatigue, frailty, immobilization, inflammatory myositis, malnutrition, metabolic syndrome, neuromuscular disease, obesity, post-surgical muscle weakness, post-traumatic muscle weakness, sarcopenia, and toxin exposure.

In some embodiments, the composition comprises an additional active agent (e.g., pharmaceutically active agent). In addition to the active agents described herein, non-limiting examples of suitable additional active agents include agents that increase skeletal muscle mass, promote skeletal muscle recovery, promote skeletal muscle anabolism, treat or prevent muscle atrophy or disorders characterized by muscle atrophy.

The compositions of the present disclosure may also comprise at least one excipient. Non-limiting examples of suitable excipients include a buffering agent, a preservative, a stabilizer, a binder, a compaction agent, a lubricant, a dispersion enhancer, a disintegration agent, a flavoring agent, a sweetener, a coloring agent, and combinations of any of these agents.

In some embodiments, the excipient comprises a buffering agent. Non-limiting examples of suitable buffering agents include sodium citrate, magnesium carbonate, magnesium bicarbonate, calcium carbonate, and calcium bicarbonate. In some embodiments, the excipient comprises a preservative. Suitable examples of preservatives include antioxidants, such as alpha-tocopherol or ascorbate, and antimicrobials, such as parabens, chlorobutanol, or phenol.

In some embodiments, the excipient comprises a binder. Suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof. In some embodiments, the excipient comprises a lubricant. Suitable non-limiting examples of lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the excipient comprises a dispersion enhancer. Suitable dispersants may include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the excipient comprises a disintegrant. The disintegrant may be a non-effervescent disintegrant. Suitable examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. The disintegrant may be an effervescent disintegrant. Suitable effervescent disintegrants include sodium bicarbonate in combination with citric acid, and sodium bicarbonate in combination with tartaric acid.

In some embodiments, the excipient comprises a flavoring agent. Flavoring agents (e.g., incorporated into the outer layer) may be chosen from synthetic flavor oils and flavoring aromatics; natural oils; extracts from plants, leaves, flowers, and fruits; and combinations thereof. For example, these may include cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the excipient comprises a coloring agent. Suitable color additives include food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in certain embodiments.

The weight fraction of the excipient or combination of excipients in the formulation may be about 30% or less, about 25% or less, about 20% or less, about 15% or less, about 10% or less, about 5% or less, about 2%, or about 1% or less of the total weight of the amino acid composition.

Also contemplated are methods relating to delivery of the compositions disclosed herein, including but not limited to dosage form and route of administration. The compositions disclosed may be formulated into any suitable form and administered by any suitable means. For example, the compositions may be administered orally, rectally, or parenterally, in formulations containing conventionally acceptable carriers, adjuvants, and vehicles as desired. The term “parenteral” as used herein includes subcutaneous, intravenous, intramuscular, or intrasternal injection, or infusion techniques. In an exemplary embodiment, the agents or compositions comprising the agents disclosed herein are administered orally.

Solid dosage forms for oral administration may include capsules, tablets, caplets, pills, troches, lozenges, powders, and granules. A capsule typically comprises a core material comprising a composition of the invention and a shell wall that encapsulates the core material. The core material may be solid, liquid, or an emulsion. The shell wall material may comprise soft gelatin, hard gelatin, or a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate; vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Some such polymers may also function as taste-masking agents.

Tablets, pills, and the like may be compressed, multiply compressed, multiply layered, and/or coated. In some embodiments, the coating material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In some embodiments, the coating material comprises a protein. Suitable proteins include, but are not limited to, gelatin, casein, collagen, whey proteins, soy proteins, rice protein, and corn proteins. In some embodiments, the protein is not gelatin. In some embodiments, the protein is not casein, in some embodiments, the protein is not collagen. In some embodiments, the protein is not whey protein. In some embodiments, the protein is not soy protein. In some embodiments, the protein is not rice protein. In some embodiments, the protein is not corn protein. In some embodiments, the coating material comprises a fat or oil, and in particular, a high melting temperature fat or oil. The fat or oil may be hydrogenated or partially hydrogenated, and preferably is derived from a plant. The fat or oil may comprise glycerides, free fatty acids, fatty acid esters, or a mixture thereof. In some embodiments, the coating material comprises an edible wax. Edible waxes may be derived from animals, insects, or plants. Non-limiting examples include beeswax, lanolin, bayberry wax, carnauba wax, and rice bran wax. Tablets and pills may additionally be prepared with enteric coatings.

Alternatively, powders or granules embodying the compositions disclosed herein may be incorporated into a food product. In some embodiments, the food product may be a drink for oral administration. Non-limiting examples of a suitable drink include fruit juice, a fruit drink, an artificially flavored drink, an artificially sweetened drink, a carbonated beverage, a sports drink, a liquid diary product, a shake, and so forth. Other suitable means for oral administration include aqueous and nonaqueous solutions, emulsions, suspensions and solutions and/or suspensions reconstituted from non-effervescent granules, containing suitable solvents, preservatives, emulsifying agents, suspending agents, diluents, sweeteners, coloring agents, and flavoring agents.

The food product may also be a solid foodstuff. Suitable examples of a solid foodstuff include a food bar, a snack bar, a nutrition bar, a cookie, a brownie, a muffin, a cracker, an ice cream bar, a frozen yogurt bar, and the like.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Example 1

### In Vitro Studies

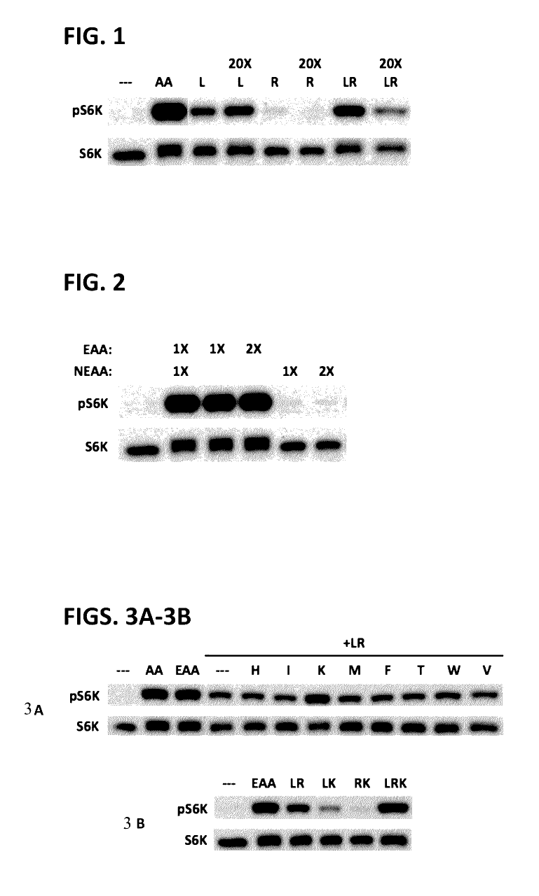

The work described herein determined which amino acids are most critical for signaling to the mTORC1 pathway in HEK-293T cells, the workhorse cell culture system to which we owe much of our understanding about amino acid sensing. The basic experimental paradigm consists of a 50-minute total amino acid starvation followed by a 10-minute stimulation with a customizable set of amino acids. It has been reported in the literature that omission of either leucine (Leu) or arginine (Arg) from cell culture media suppresses mTORC1 kinase activity as denoted by the phosphorylation state of mTORC1 substrate S6K.⁸ This result suggests that both Leu and Arg are necessary for mTORC1 activation. However, the work described herein demonstrates that stimulating 293T cells with Leu and Arg together is not sufficient to induce complete mTORC1 activation, a state defined by the phospho-S6K levels achieved upon stimulation with total amino acids at 1×RPMI levels (FIG. 1), which reflect amino acid concentrations in human plasma⁹. The work described herein determined that that there may be one or more missing amino acids that, when added alongside Leu and Arg, could fully activate mTORC1.

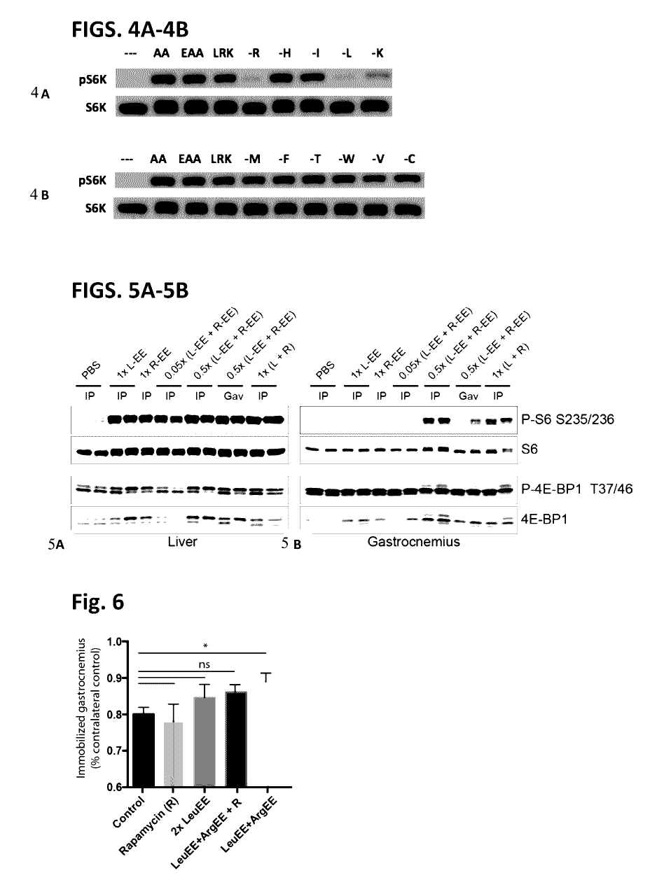

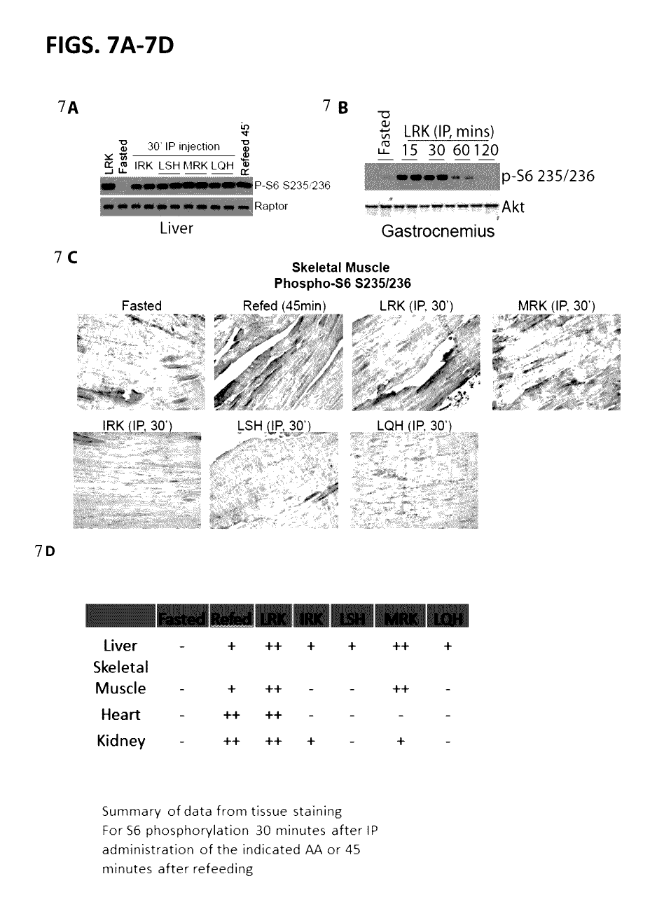

To determine the identities of these missing amino acids, the inventors first stimulated cells with the set of essential or the set of nonessential amino acids. The inventors noticed that 1×essential amino acids (EAA), which contain both Leu and Arg, activated mTORC1 to the same degree as 1× total amino acids (FIG. 2). By contrast, 1×non-essential amino acids (NEAA) had no activating effect at all (FIG. 2). The inventors then tested triplet amino acid combinations comprised of Leu, Arg, and one additional essential amino acid. Surprisingly, the combination of Leu, Arg, and Lys (LRK) was sufficient to match the stimulatory effect of total amino acids (FIG. 3). Cocktails of EAAs that omitted one amino acid at a time were prepared. Interestingly, the only cocktails that failed to fully activate mTORC1 lacked Leu, Arg, or Lys; omission of no other essential amino acid affected phospho-S6K levels (FIG. 4). Thus the necessity and sufficiency experiments yielded complementary results, converging on Leu, Arg, and Lys as key signals for mTORC1 activation in 293T cells.

The inventors have explored why this particular combination of amino acids has such synergistic effects on the mTORC1 pathway in 293T cells. Given that both Arg and Lys contain basic side chains, the inventors first wondered if this shared chemical property underlay their abilities to activate mTORC1. However, the inventors found that Arg and Lys cannot substitute for each other in the LRK stimulation setting: neither replacing Lys with a second equivalent of Arg (LRR) nor replacing Arg with a second equivalent of Lys (LKK) approached the activating ability of LRK.

Furthermore, the inventors continue to characterize structure-activity relationships using LRK derivatives with modifications on the carboxy terminus, the amino terminus, and/or the side chain in whole-cell and in vitro lysosomal assays of mTORC1 activity. The inventors first tested amino acid carboxyesters, whose enhanced ability to permeate membranes render them good candidates for lysosomal loading. Once inside the lysosome, amino acid esters are thought to undergo hydrolysis to yield their native counterparts. Methyl- and ethyl esters of leucine and lysine are modestly more stimulatory than the corresponding native amino acid in whole-cell assays; however, unexpectedly, arginine esters are less potent than arginine itself. Further preliminary evidence suggests that Leu modified by an amino Cbz protecting group retains the ability to signal to mTORC1, albeit with lower potency than native Leu. This result suggests that the amino terminus of Leu may tolerate a bulky substituent without complete ablation of mTORC1 signaling ability. The inventors have also observed that photo-crosslinkable Leu with a diazirine-modified side chain retains some mTORC1 stimulatory activity. Knowledge of the chemical space around LRK can be used to develop probes for interrogating the LRK/protein(s) interaction interface for isolating and/or enriching the amino acid sensor(s) feeding into mTORC1.

The experiments described above demonstrate that a highly reduced set of amino acids sufficient for fully activating mTORC1 exists.

### Example 2

### In Vivo Studies

The discovery of LRK as rapid, strong and long-lasting activators of mTORC1 in tissue culture, prompted us to evaluate the efficacy of such intervention in the context of a mammalian organism. We performed in mice the equivalent experiment to that of amino acid deprivation and re-stimulation in cultured cells; this involves a prolonged fasting, which causes a drop in circulating amino acids, and then the intra-peritoneal (IP) injection of the desired combination of amino acids. Due to limited solubility of native amino acids for injections in mice, we also tested the effect of esterified amino acids, which have been proven to potently activate mTORC1 in cultured cells⁵.

As observed in FIG. 5, fasting induces a drop in mTORC1 activation in mouse liver and skeletal muscle, as determined by decreased phosphorylation of mTORC1 effector molecules S6 and 4EBP1. Upon re-feeding, injection of LR amino acids or amino acid ethyl esters, mTORC1 is rapidly reactivated. For the purpose of these experiments, an amino acid dose equivalent to 20% of daily intake, and defined as “X,” was used to study anabolic signaling in mice. In the case of amino acid esters equimolar AA concentrations were calculated for each treatment. These values were chosen to roughly reflect substantial nutrient intake that occurs following an overnight fast. Interestingly, liver mTORC1 activity is promoted in response to administration of single and combination amino acid treatments whereas skeletal muscle mTORC1 activity is significantly induced in response to the LR combination only. Moreover, the effect of LR ethyl esters on skeletal muscle mTORC1 activity is 1) more potent and 2) longer lasting when compared to the effect of the same combination of native amino acids (FIG. 5 and data not shown). This validates the discovery in cultured cells and provides a proof of concept support for the use of this intervention to activate mTORC1 and its downstream anabolic response in mammals. Furthermore, these findings suggest a qualitative benefit from the LR ethyl ester combination, in comparison with other treatments previously investigated.

In other words, a combination of amino acid derivatives necessary and sufficient to activate mTORC1 in skeletal muscle constitutes a potential intervention to promote skeletal muscle anabolism, a desirable goal in several clinical conditions. These conditions include aging, denervation or immobilization, cachexia, and nutrient limitations, which are all scenarios where muscle atrophy and/or waste causes morbidity and can also be life threatening. In these situations, artificial mTORC1 activation has shown promise, albeit limited, in protecting from skeletal muscle waste. Administration of leucine has been used in most cases, and proven a partial benefit. Given the substantial effect of LR ethyl esters on mTORC1 activity compared to L alone, we aimed to test this combination in a hindlimb immobilization model of muscle atrophy and waste.

The protocol of hindlimb immobilization involves casting of the rear left leg, resulting in atrophy of the gastrocnemius and soleus muscles. The rear right leg remains free, and serves as an internal control for gastrocnemius and soleus muscle mass. Following one week of hindlimb immobilization skeletal muscles are significantly lighter, with thinner fibers having undergone significant autophagic and proteolytic degradation. Upon removal of the cast, hindlimb muscles are able to contract and extend, and muscle mass recovery occurs. mTORC1 activation protects both from atrophy and enhances recovery, and leucine has shown some success in boosting mTORC1 activation and beneficial effects on muscle physiology upon immobilization/recovery insults.

The work described herein demonstrates that LR ethyl esters can exert a significant improvement in muscle recovery upon immobilization atrophy. The inventors performed an experiment where left hindlimbs were immobilized for 5 days, casts were removed, and a recovery period of 5 days continued until the study endpoint. Animals were injected with PBS as control, L ethyl ester, or LR ethyl esters twice daily. As in the previous experiment, the 1× doses of amino acids and their esters were calculated as 20% of the amount that an adult mouse eats in a 24 h period under ad libitum conditions. Also included were daily treatments with the mTORC1 inhibitor rapamycin (rapamycin alone and rapamycin+LR ethyl ester) to determine whether the effects of the amino acids were dependent on mTORC1 activity. FIG. 6 shows relative gastrocnemius muscle mass following the hindlimb immobilization study. Strikingly, compared to control (PBS injected mice) the only treatment that led to a significant increase in gastrocnemius muscle mass was LR ethyl ester treatment. This demonstrates that the LR combination of amino acid derivatives exerts a significant effect on skeletal muscle mass, dependent on mTORC1 activity, and can be considered a promising approach to benefit patients suffering from muscle waste. Without wishing to be bound by theory, it is believed that the LR ethyl esters achieved the above results in the absence of administering K ethyl ester because endogenous K levels in the animals were sufficiently high in the absence of administration of K ethyl ester. Without wishing to be bound by theory, the effect of LR ethyl esters may be further enhanced by administration of K ethyl ester or other K mimetics.

### Example 3

### Assaying Analogs for mTORC1 Modulation

Analogs of leucine (L), arginine (R), and lysine (K) can be assayed to determine whether they are capable of activating (e.g., agonists) or suppressing (e.g., antagonists) mTORC1 activity. Generally, to test analogs of L, R, and K, whole cells (e.g., HEK293) can be starved of all amino acids (using RPMI—aa) for a period of time (e.g., about 50 min), and then stimulated with a combination of L, R, or K and/or an analog of L, R, or K, and mTORC1 activity can be measured by phosphorylation of an mTORC1 substrate (e.g., S6K).

Below are exemplary conditions which can be used to assay analogs of leucine (e.g., photoleucine (photoLeu)) for their ability to activate or suppress mTORC1 activity.

### 1) No AA

2) essential AA

### 3) RK

4) RK+0.1× photoLeu
5) RK+0.5× photoLeu
6) RK+1× photoLeu
7) RK+10× photoLeu

### 8) LRK

9) LRK+0.1× photoLeu
10) LRK+0.5× photoLeu
11) LRK+1× photoLeu
12) LRK+10× photoLeu

Conditions 3-7 above test whether or not a leucine analog (e.g., photoLeu) can substitute for native Leu. If the pS6K signal increases, as in the case of photoLeu, the analog assayed is considered an agonist or activator of the mTORC1 pathway. Those skilled in the art will appreciate that conditions 3-7 above can be modified to assay other analogs, for example, by replacing RK with LK to assay R analogs or by replacing RK with LR to assay K analogs.

Conditions 8-12 above test whether or not a leucine analog (e.g., photoLeu) can compete against native Leu. If the pS6K signal caused by baseline LRK is not suppressed with increasing amounts of leucine analog, as in the case of photoLeu, the leucine analog (e.g., photoLeu) is not an antagonist or mTORC1-inactivating competitor against native Leu. Those skilled in the art will appreciate that conditions 8-12 above can be modified to assay other analogs, for example, by replacing the leucine analog (e.g., photoLeu) with an arginine or lysine analog.

The concentration range of analogs used in the protocol set forth above was from about 0.1× to 10× (relative to RPMI), however, other concentrations may be used, as will be appreciated by the skilled artisan.

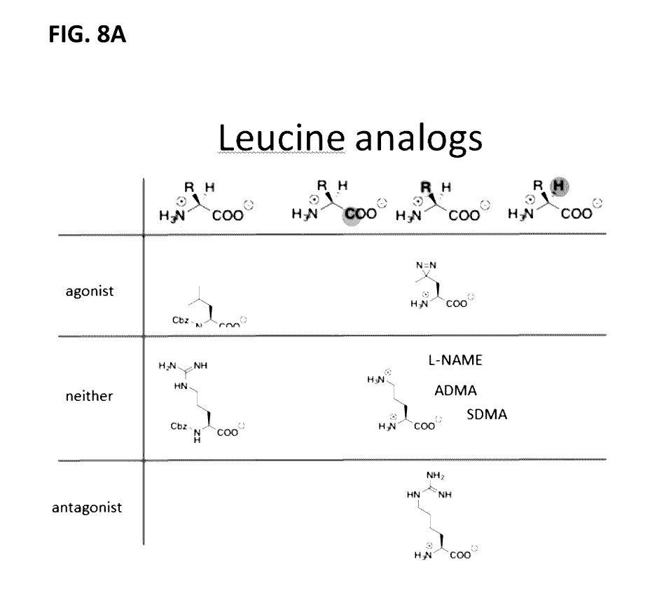

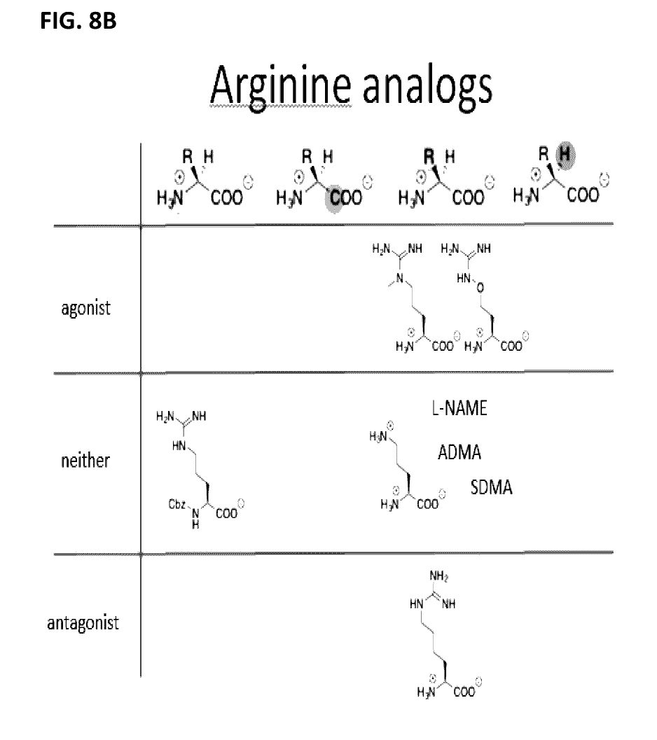

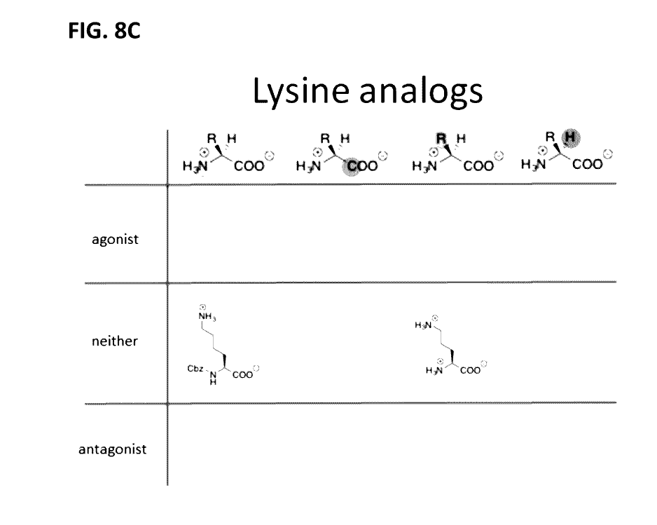

Exemplary analogs which were tested in accordance with the protocol described above are shown in FIGS. 8A (leucine analogs), 8B (arginine analogs), and 8C (lysine analogs).

### Example 4

### Rag GTPase-Mediated Regulation of mTORC1 by Amino Acids and Glucose is Necessary for Neonatal Autophagy and Survival

The mechanistic target of rapamycin (mTOR) is a serine-threonine kinase that as part of mTOR complex 1 (mTORC1) regulates anabolic and catabolic processes required for cell growth and proliferation (Sabatini, et al. Nat Rev Mol Cell Biol 12, 21-35 (2010)). mTORC1 integrates signals that reflect the nutritional status of an organism and senses growth factors and nutrients through distinct mechanisms. Growth factors regulate mTORC1 via the PI3K/Akt/TSC1-TSC2 axis, while amino acids act through the Rag family of GTPases (Kim, et al. Nat Cell Biol 10, 935-945 (2008); Sancak, Y. et al. Science 320, 1496-1501 (2008)). When activated, these GTPases recruit mTORC1 to the lysosomal surface, an essential step in mTORC1 activation (Sancak et al. 2008; Sancak, et al. Cell 141, 290-303 (2010)). Amino acid levels regulate nucleotide binding to the Rag GTPases in a Ragulator- and vacuolar-type H-ATPase-dependent manner (Sancak, et al. 2010; Zoncu, et al. Science 334, 678-683 (2011)). In the absence of amino acids, RagA (or RagB, which acts in an identical manner) is loaded with GDP, but becomes bound to GTP when amino acids are plentiful.

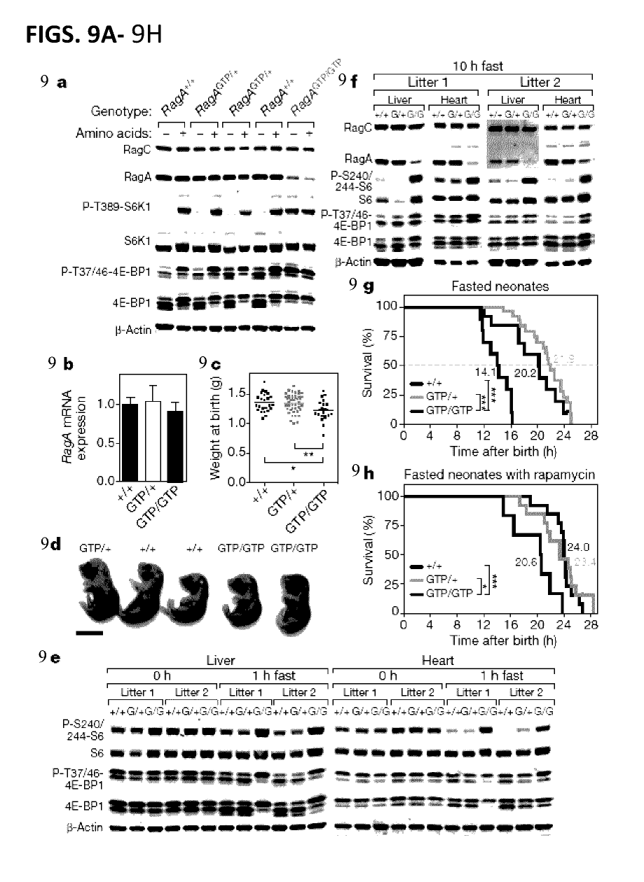

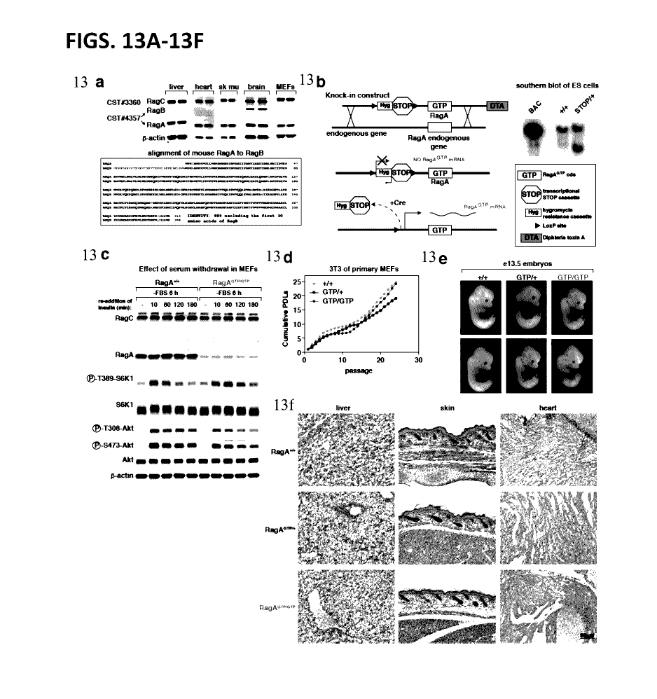

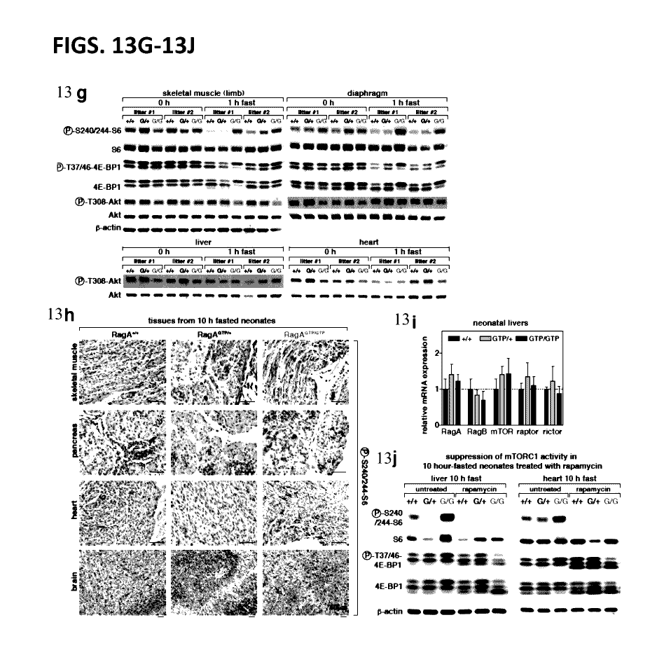

To study the physiological importance of the amino acid-dependent activation of mTORC1, we generated knock-in mice that express a constitutively active form of RagA. We chose to manipulate RagA because, although highly similar to RagB, RagA is much more abundant and widely expressed than RagB in mice (FIG. 13A). By a single nucleotide substitution in the RagA coding sequence, we replaced glutamine in position 66 with leucine, generating a RagA mutant (RagA^{Q66L}) (FIG. 13B) that is, regardless of amino acid levels, constitutively active, mimicking a permanent GTP-bound state (Sancak et al. 2008; Hirose, et al. J Cell Sci 111 (Pt 1), 11-21 (1998)) (hereafter referred to as RagA^{GTP}). We obtained mouse embryo fibroblasts (MEFs) from E13.5 embryos and evaluated mTORC1 signaling upon amino acid or serum deprivation. In RagA⁺⁺ and RagA^{GTP/+} cells, deprivation of either amino acids (FIG. 9A) or serum (FIG. 13C) suppressed mTORC1 activity, as determined by phosphorylation state of the mTORC1 substrates S6K1 and 4E-BP1. In contrast, in RagA^{GTP/GTP} cells, mTORC1 activity was completely resistant to amino acid withdrawal (FIG. 9A). However, regulation of PI3K activity by serum was intact, as reflected by Akt phosphorylation (FIG. 13C). Interestingly, RagA protein levels were reduced in RagA^{GTP/GTP} cells, but this was not a consequence of lower RagA^{GTP} mRNA expression (FIG. 9B), supporting the existence of a negative feedback triggered by RagA activity. Nevertheless, the cells show the expected amino acid-independent activation of mTORC1.

Cells lacking the TSC1-TSC2 tumor suppressor complex also have deregulated mTORC1 activity as such cells maintain mTORC1 signaling in the absence of growth factors¹. Unlike TSC1- or TSC2-deficient MEFs (Kwiatkowski, et al. Hum Mol Genet 11, 525-534 (2002); Zhang, H. et al. J Clin Invest 112, 1223-1233 (2003)), RagA^{GTP/GTP} MEFs have normal proliferation rates without accelerated senescence (FIG. 13D). Furthermore, unlike TSC1- or TSC2-deficient embryos, which die at E11.5-E13.5, RagA^{GTP/GTP} embryos were indistinguishable from RagA^{+/+} embryos (FIG. 13E), and fetuses were obtained and genotyped at term with the expected Mendelian ratios from heterozygous crosses. Thus, unlike with growth factor sensing, the inability of mTORC1 to sense amino acid deprivation does not compromise survival during embryonic development, with its steady placental supply of nutrients.

Although apparently not deleterious during in utero development, constitutive RagA activity greatly impairs early postnatal survival. Heterozygous RagA^{GTP/+} mice did not have any obvious phenotypic alteration, in agreement with the normal signaling observed in RagA^{GTP/+} MEFs. However, no RagA^{GTP/GTP} mice were obtained at weaning, and were usually found dead within one day postpartum in breeding cages. Neonatal death can stem from a variety of defects, so we obtained full-term E19.5 mice by Caesarian-section and monitored them outside the breeding cage. Despite having a mild decrease in weight, RagA^{GTP/GTP} neonates were barely distinguishable from control littermates (FIGS. 9C, 9D), and histological analyses revealed no abnormalities (FIG. 13F).

To understand how constitutive RagA activity affects the regulation of mTORC1 by fasting, we compared the phosphorylation levels of S6 and 4E-BP1, established markers of mTORC1 activity, in tissues obtained from neonates at birth or fasted for 1 or 10 hours. Interestingly, just 1 hour of fasting was sufficient to strongly inhibit mTORC1 in RagA^{+/+} and RagA^{GTP/+}, but not RagA^{GTP/GTP} neonates (FIG. 9E and FIG. 13G), and this difference persisted even after 10 hours of fasting (FIG. 9F and FIG. 13H). In contrast, Akt phosphorylation was modest at birth and decreased in mice of all genotypes (FIG. 13G). As in MEFs, RagA protein levels were low in the tissues of RagA^{GTP/GTP} mice, but this again was not due to reduced mRNA levels (FIG. 13I). Collectively, these results indicate that constitutive RagA activity causes a profound defect in the response of mTORC1 to fasting.

To examine the consequences of this defect, we fasted neonates for longer periods of time, which revealed that RagA^{GTP/GTP} neonates have an accelerated time to death (˜14 hours for RagA^{GTP/GTP} versus ˜21 hours in RagA^{+/+} and RagA^{GTP/+}) (FIG. 9G). This was not the consequence of unappreciated developmental defects, as the treatment of pups at birth with the mTORC1 inhibitor rapamycin, which suppressed mTORC1 activity in all neonates (FIG. 13J), significantly delayed the death of fasted RagA^{GTP/GTP} neonates from ˜14 h to ˜21 h; p<0.01) (FIG. 9H). These data suggest that Rag-mediated regulation of mTORC1 is necessary for mice to adapt to and survive the starvation period that they endure between birth and feeding.

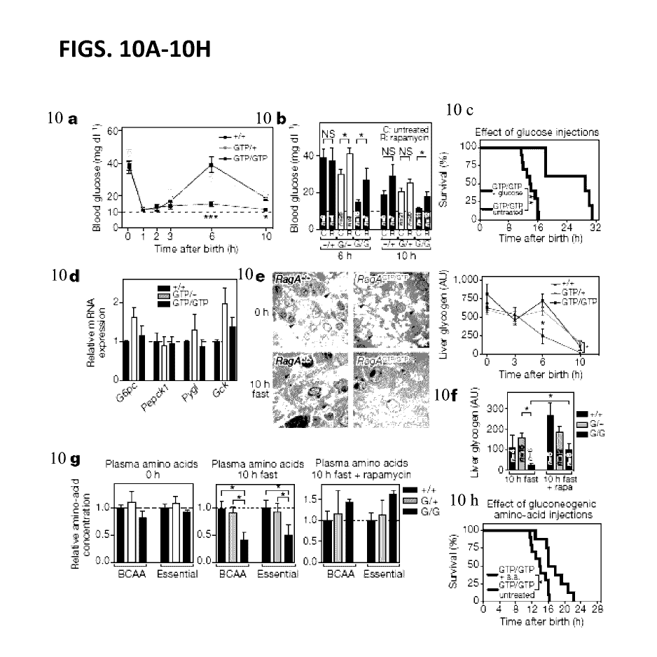

Consistent with this notion, analysis of blood glucose levels revealed that fasted RagA^{GTP/GTP} neonates suffer a profound defect in nutrient homeostasis. After one hour of fasting, glycaemia dropped dramatically in all neonates to below our 10 mg/dl limit of detection (FIG. 10A), but by 3 to 6 hours the wild-type animals restored their blood glucose to near birth levels (˜40 mg/dl). In sharp contrast, in RagA^{GTP/GTP} neonates, blood glucose levels never recovered and remained at ˜10 mg/dl or lower until death (FIG. 10A). Consistent with its rescue of the accelerated lethality of the RagA^{GTP/GTP} neonates during fasting (FIG. 9H), rapamycin administration partially reversed their defect in blood glucose levels (FIG. 10B). Moreover, injections of glucose prolonged the lifespan of fasted RagA^{GTP/GTP} mice (FIG. 10C), arguing that a lack of glucose has a causal role in their compromised survival.

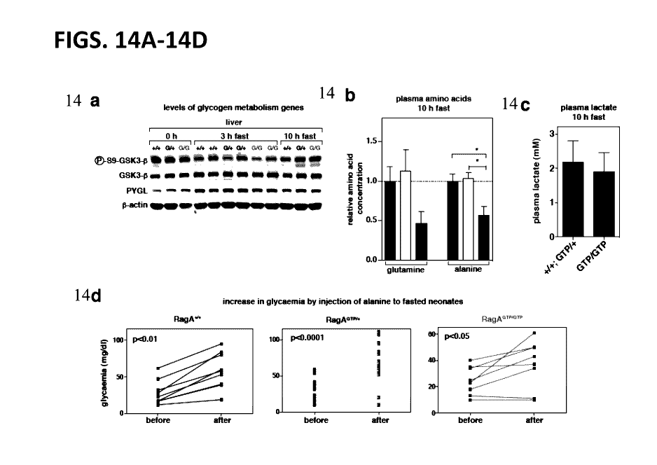

Because the inability to generate glucose from glycogen can cause perinatal lethality (Scheuner, et al. Mol Cell 7, 1165-1176 (2001)), we initially hypothesized that the RagA^{GTP/GTP} neonates had a glycogen metabolism defect. However, RagA^{GTP/GTP} neonates did not have defects in the protein or mRNA levels of the key enzymes of glycogen metabolism (FIG. 10D and FIG. 14A). Moreover, at birth RagA^{GTP/GTP} neonates had normal amounts of hepatic glycogen, which, upon fasting, they consumed at a faster rate than RagA^{+/+} and RagA^{GTP/+} animals (FIG. 10E), suggesting not a defect in its breakdown but rather accelerated use secondary to hypoglycaemia. Like with other characteristics of RagA^{GTP/GTP} mice, rapamycin administration partially restored their hepatic glycogen (FIG. 1 OF).

We also considered defects in gluconeogenesis or the availability of gluconeogenic substrates as potential reasons for the inability of RagA^{GTP/GTP} neonates to restore blood glucose levels upon fasting. Here too the RagA^{GTP/GTP} neonates did not have aberrations in the expression levels of the relevant enzymes (FIG. 10D). However, after a 10-hour fast, RagA^{GTP/GTP} neonates did have, compared to RagA^{+/+} and RagA^{GTP/+} littermates, significantly lower levels of plasma amino acids (FIG. 10G and FIG. 14B). Because murine neonates are born without significant fat stores (Birsoy, et al. Development 138, 4709-4719 (2011), lipid mobilization cannot serve as a substrate for de novo glucose production. Moreover, lactate, another substrate for gluconeogenesis, was not reduced in RagA^{GTP/GTP} neonates (FIG. 14C), arguing for a specific reduction in amino acid substrates. As with glucose levels and glycogen stores (FIGS. 10B, 10F), rapamycin administration reversed the decrease in amino acids levels in RagA^{GTP/GTP} neonates (FIG. 10G). Furthermore, injection of a mix of gluconeogenic amino acids, which can contribute to gluconeogenesis but not protein synthesis, delayed the onset of death of RagA^{GTP/GTP} neonates (FIG. 10H), and injection of just alanine to fasted neonates provoked a significant increase in glycaemia (FIG. 14D). These data are consistent with the glucose homeostasis defect of the fasted RagA^{GTP/GTP} neonates being a consequence of reduced circulating amino acids, which leads to lower de novo glucose production and plasma levels, and accelerated death.

Several properties of the RagA^{GTP/GTP} mice are reminiscent of autophagy-deficient mice (Kuma, et al. Nature 432, 1032-1036 (2004); Komatsu, et al. J Cell Biol 169, 425-434 (2005)), including the reduction in plasma amino acids and lifespan upon fasting, as well as the slightly lower birth weight. Although mTORC1 negatively regulates autophagy (Mizushima, et al. Nature 451, 1069-1075 (2008)), and amino acid levels are regulators of autophagy in rats (Mortimore, G. E. & Schworer, C. M. Nature 270, 174-176 (1977), many mTORC1-dependent and mTORC1-independent autophagy regulators exist (Kroemer, G., Marino, G. & Levine, B. Mol Cell 40, 280-293 (2010)). Hence, we wondered if perturbing just one of the several inputs to mTORC1 could exert a dominant effect in the physiological regulation of autophagy.

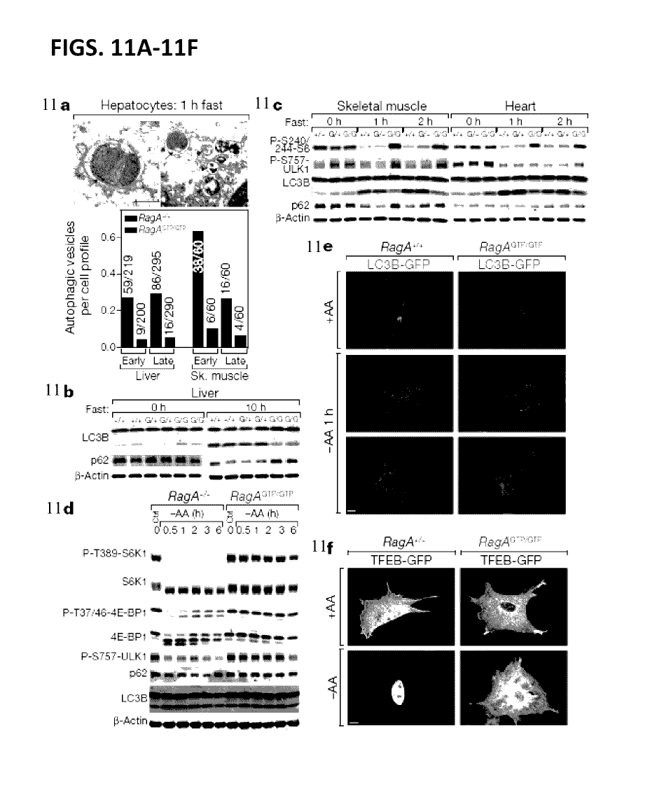

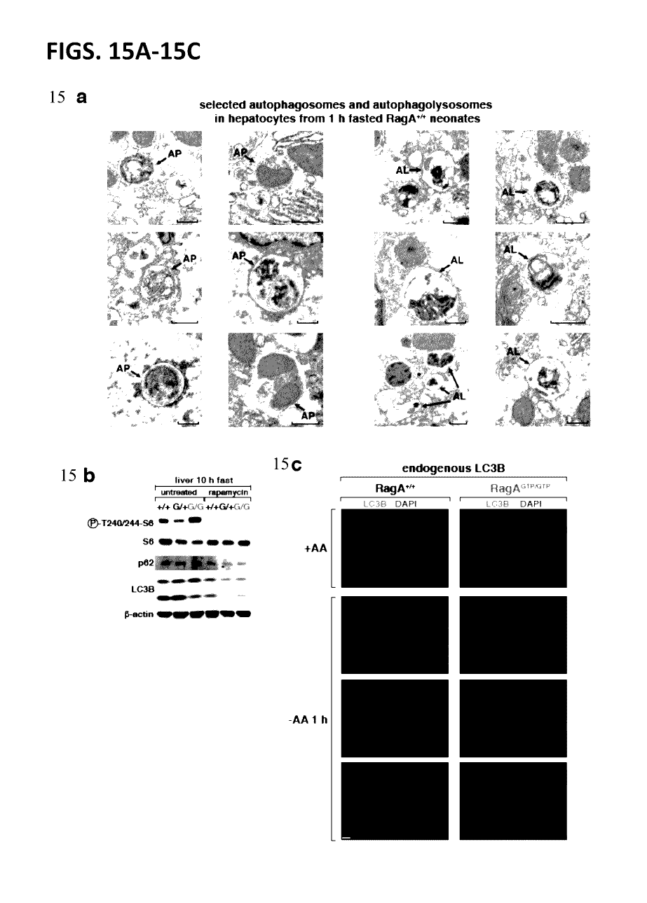

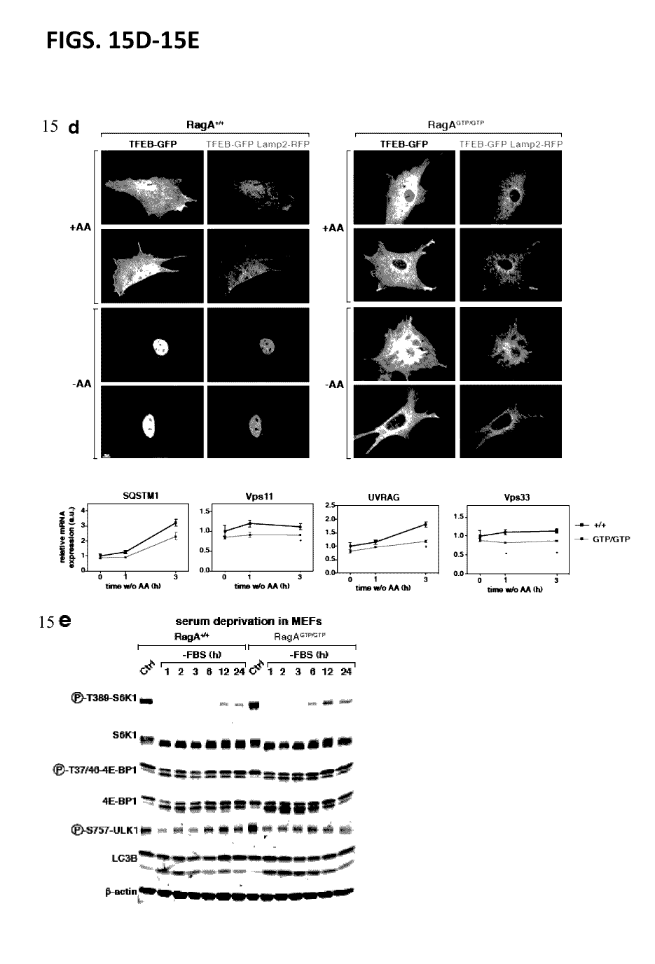

Quantitative electron microscopic analyses of livers from one-hour fasted RagA^{+/+} neonates revealed abundant autophagosomes, characterized by double limiting membranes (FIG. 11A and FIG. 15A). Autophagosomes rapidly mature into single-membrane autophagolysosomes, so these were also found in RagA^{+/+} livers (FIG. 11A and FIG. 15A), albeit the ratio of autophagosomes to autophagolysosomes was high. Importantly, both autophagic vacuoles were rarely observed in fasted RagA^{GTP/GTP} littermates (FIG. 11A). Similar results were obtained when skeletal muscle was analyzed (FIG. 11A).

Even after 10 hours of fasting, the autophagy defect in the livers of RagA^{GTP/GTP} neonates persisted, as detected by the reduced cleavage of LC3B and degradation of p62 (FIG. 11B), which was increased by administration of rapamycin (FIG. 15B). Biochemical analyses for these markers in skeletal and cardiac muscles from RagA^{GTP/GTP} neonates after 1 and 2 h of fasting were also consistent with impaired autophagy (FIG. 11C). Cells in culture mirrored the in vivo findings (FIG. 11D), and these results were confirmed by detection of LC3B localization using fluorescence microscopy in amino acid-starved cells (FIG. 11E and FIG. 15C). Consistently, phosphorylation of the autophagy activator ULK-1, a direct substrate of mTORC1 that was suppressed in RagA^{+/+} MEFs upon amino acid withdrawal, remained high in RagA^{GTP/GTP} cells (FIG. 11D). In addition, we looked at the transcription factor TFEB, which upregulates genes involved in lysosomal biogenesis and autophagy, but is excluded from the nucleus when phosphorylated by mTORC1 (Roczniak-Ferguson, A. et al. Science signaling 5, ra42 (2012); Settembre, C. et al. EMBO J(2012)). Upon amino acid deprivation, TFEB localized to the nuclei of RagA^{+/+} but not RagA^{GTP/GTP} MEFs (FIG. 11F and FIG. 15D). This result was mirrored by the decreased expression of TFEB transcriptional targets (Supplementary FIG. 3d).

Serum withdrawal, which inhibits mTORC1 in a Rag-independent fashion, suppressed mTORC1 activity and triggered autophagy in MEFs of all genotypes (FIG. 15E), indicating that constitutive RagA activity does not block autophagy induction by all signals. Thus, despite the multitude of pathways that regulate autophagy (Kroemer et al 2010), Rag GTPase activity upstream of mTORC1 is a major regulator of autophagy in vivo during the perinatal period.

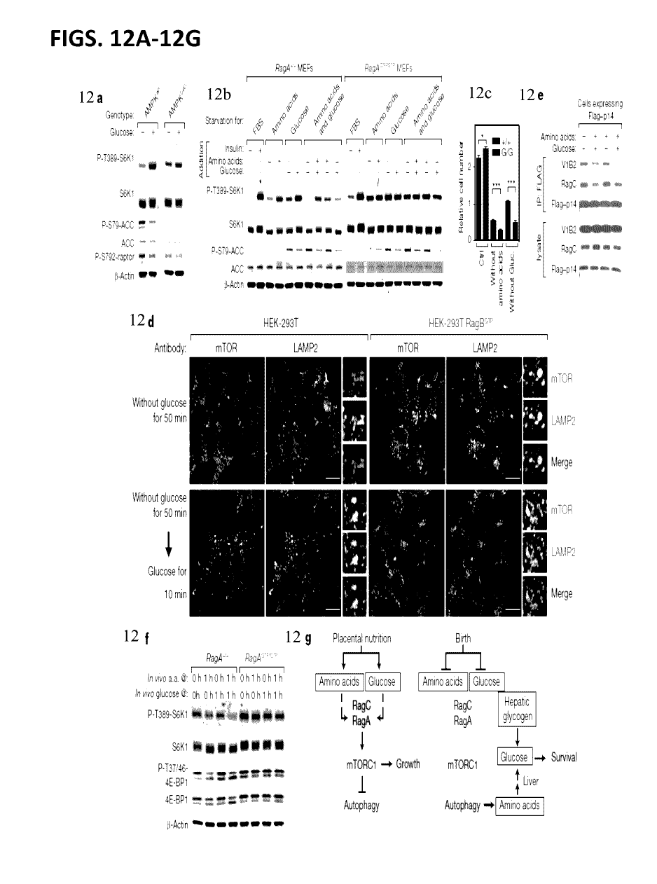

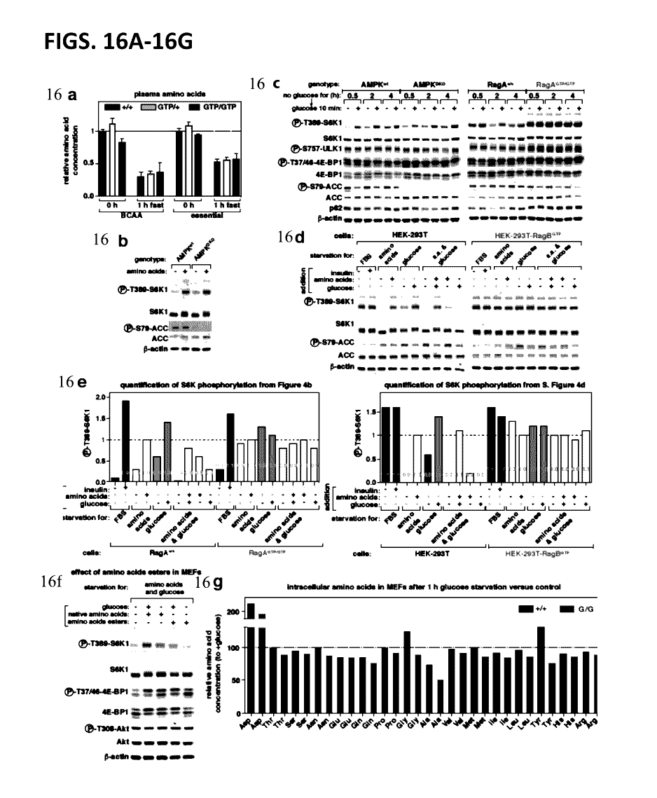

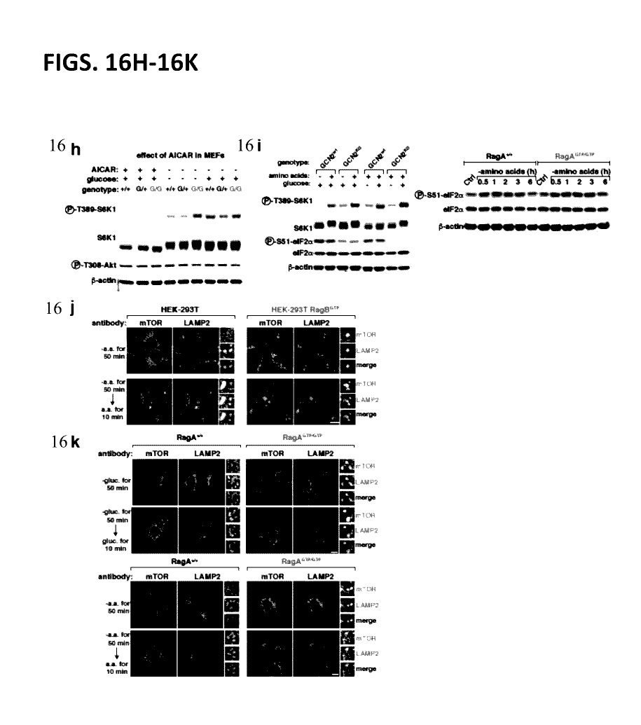

Maintenance of mTORC1 activity requires the simultaneous presence of growth factors, amino acids, and glucose¹. We found that just one hour of fasting, both plasma amino acid and glucose levels were reduced in neonates of all genotypes (FIG. 10A and FIG. 16A). The drop in nutrient levels correlated with a strong inhibition of mTORC1 activity in RagA^{+/+} and RagA^{GTP/+}, but not RagA^{GTP/GTP} neonates (FIG. 1e). Thus, despite a profound hypoglycemic state, mTORC1 activity remained high in fasted RagA^{GTP/GTP} neonates, a puzzling result given that the Rag GTPases are thought to have a specialized role in amino acid sensing. These observations led us to hypothesize that the Rag GTPases participate in the direct sensing of glucose levels, in addition to their established role in amino acid sensing. A well-established link between low glucose (but not amino acids [FIG. 16B]) and mTORC1 inhibition is the AMP-activated protein kinase (AMPK). However, in MEFs lacking AMPK α1 and α2 (AMPK-DKO), mTORC1 activity was still repressed upon glucose deprivation, albeit less prominently than in wild-type MEFs (FIG. 12A). This indicates that an AMPK-independent pathway of mTORC1 inhibition exists, as shown recently in the context of metformin treatment (Kalender, A. et al. Cell Metab 11, 390-401 (2010)). Compared to control cells, mTORC1 signaling was largely resistant to glucose deprivation in RagA^{GTP/GTP} MEFs (FIG. 12B and FIGS. 16C, 16E) and HEK-293T cells expressing RagB^{GTP} (FIGS. 16D, 16E). It is unlikely that glucose indirectly inhibits mTORC1 by preventing amino acid transport, because amino acid esters, which freely enter cells and substitute for amino acids in mTORC1 activation s, did not substitute for glucose (FIG. 16F). Moreover, intracellular amino acid levels were only marginally affected in cells deprived of glucose (FIG. 16G). In addition, like AMPK-deficient cells (Choo, A. Y. et al. Mol Cell 38, 487-499 (2010); Shaw, R. J. et al. Proc Natl Acad Sci USA 101, 3329-3335 (2004)), RagA^{GTP/GTP} cells had enhanced sensitivity to long-term glucose deprivation-induced death (FIG. 12C). Constitutive RagA activity does not block AMPK action as aminoimidazole carboxamide ribonucleotide (AICAR), an AMPK activator, inhibited mTORC1 in cells of all genotypes (FIG. 16H). In addition, AMPK activity, as monitored by acetyl-CoA carboxylase (ACC) phosphorylation, was induced to similar levels in glucose-deprived RagA^{+/+} and RagA^{GTP/GTP} cells (FIG. 12B and FIG. 16C), but absent in AMPK-null cells (FIG. 12A). Another cellular nutrient sensor is GCN2 (Proud, C. G. Semin Cell Dev Biol 16, 3-12 (2005), but although it was regulated by amino acids, it was not by glucose; also, loss of GCN2 did not affect the inhibition of mTORC1 caused by amino acid or glucose starvation (FIG. 16I).

Amino acids promote the Rag-dependent translocation of mTORC1 to the lysosomal surface, a necessary event for its activation (Sancak et al. 2010). Interestingly, glucose deprivation, like that of amino acids (FIG. 16J), rendered mTORC1 diffusely localized in the cytoplasm of HEK-293T cells and, within minutes of glucose re-addition, mTORC1 re-clustered at lysosomes (FIG. 12D). However, in HEK-293T cells expressing RagB^{GTP} and in RagA^{GTP/GTP} TP MEFs, mTORC1 localized at the lysosomal surface regardless of glucose levels (FIG. 12D and Supplementary FIG. 16K). The lysosomal v-ATPase, necessary for the Rag-dependent activation of mTORC1 by amino acids, engages in amino acid-sensitive interactions with the Ragulator (Zoncu et al. 2011), and we found that glucose also regulates the binding of the v-ATPase to Ragulator (FIG. 12E), suggesting a shared regulatory mechanism. Finally, when amino acid and glucose concentrations at birth and after 1 hour neonatal fasting were reproduced in the in vitro culture medium, mTORC1 activity was suppressed in RagA^{+/+} but not in RagA^{GTP/GTP} cells placed under the one-hour fasting conditions (FIG. 12F). Hence, we propose that the Rag GTPases are a ‘multi-input nutrient sensor’, upon which amino acids and glucose converge, in a v-ATPase-dependent manner, upstream of mTORC1.

Altogether, our results support a chain of events that start with the interruption of maternal nutrient supply at birth, which inhibits mTORC1 presumably by converging negative inputs from profound hypoglycaemia and a drop in plasma amino acids, in a Rag-dependent fashion. During the period between birth and suckling, mTORC1 inhibition triggers autophagy, which generates the amino acids used to sustain plasma glucose levels via gluconeogenesis. Constitutive RagA activity prevents mTORC1 inhibition, leading to defective autophagy and, thus, insufficient amino acid production. The lower levels of gluconeogenic amino acids reduces hepatic generation of glucose, which accelerated glycogen breakdown fails to compensate, ultimately leading to hypoglycaemia, energetic exhaustion, and accelerated neonatal death (FIG. 12G). Thus, the Rag GTPases have a critical role in nutrient sensing by mTORC1 and in neonatal survival during fasting.

### Example 5

### Effect of Intraperitoneal Administration of Various Amino Acids on Muscle mTORC1 Activation in Immobilized and Fasted Mice

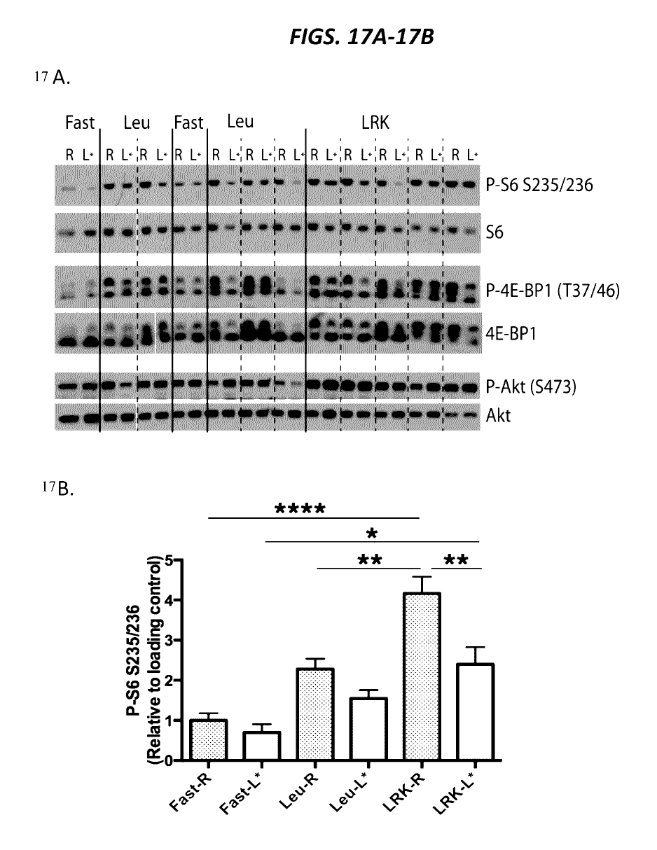

Mice were subjected to a model of hindlimb immobilization for 5 days in which the left hindlimb was casted as described in Example 2 in order to induce atrophy. Food and water were provided ad libitum. The night before day 6, food was withdrawn and the mice were subjected to an 18-hour overnight fast. On day 6 mice received an IP injection of vehicle (fasted), 1×L-Leucine, or 1× (L-Leucine, L-Arginine) plus 0.1× L-Lysine for 30 mins. Right (R; control) and left (L*; immobilized) gastrocnemius muscles were collected 30 minutes after treatment, snap frozen in liquid nitrogen, and processed for western blotting analysis with appropriate antibodies as previously described. The results are shown in FIGS. 17A and 17B.

As evident by levels of phosphorylated S6 and 4EBP1, IP administration of leucine was unable to increase mTORC1 activity in the immobilized (L*) gastrocnemius muscle in four of the 5 mice compared to fasted controls. Leucine administration stimulated mTORC1 in the non-immobilized muscle in all mice (R). In contrast, IP administration of LRK increased mTORC1 activity in the immobilized and non-immobilized muscles in 4 out of the 5 mice tested (FIG. 17A). Quantification of the average ratio of phosphorylated S6 to total S6 is represented in FIG. 17B and indicates that LRK increases mTORC1 in immobilized gastrocnemius muscles to a greater degree than leucine alone.

### Example 6

### Effect of Intraperitoneal Administration of Various Amino Acids on Muscle mTORC1 Activation and Muscle Leucine Levels in Fasted Mice

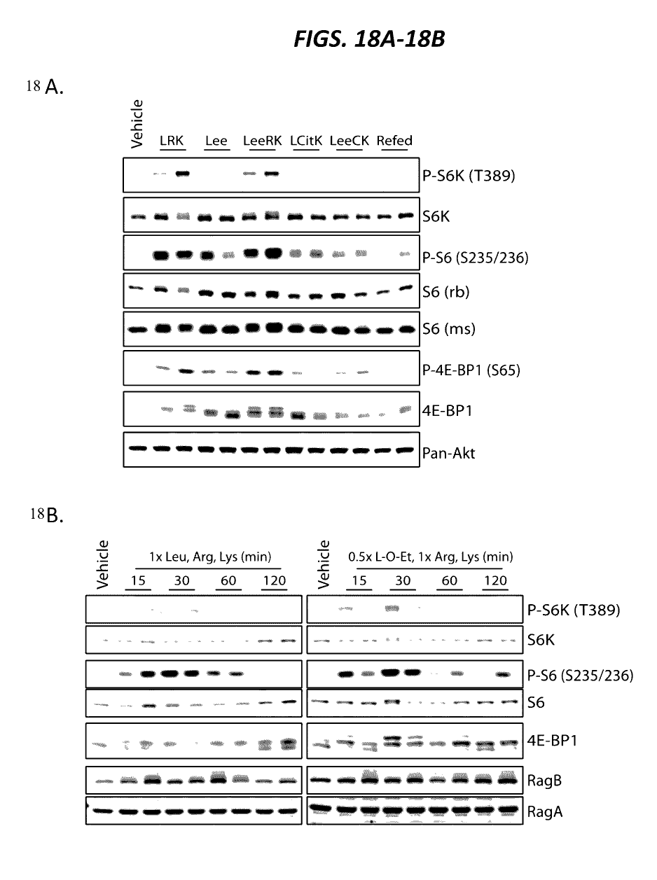

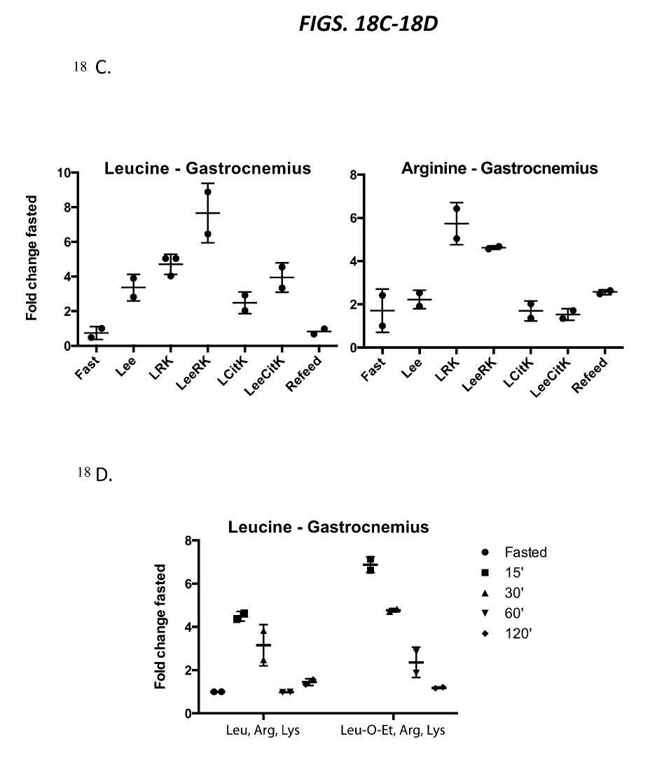

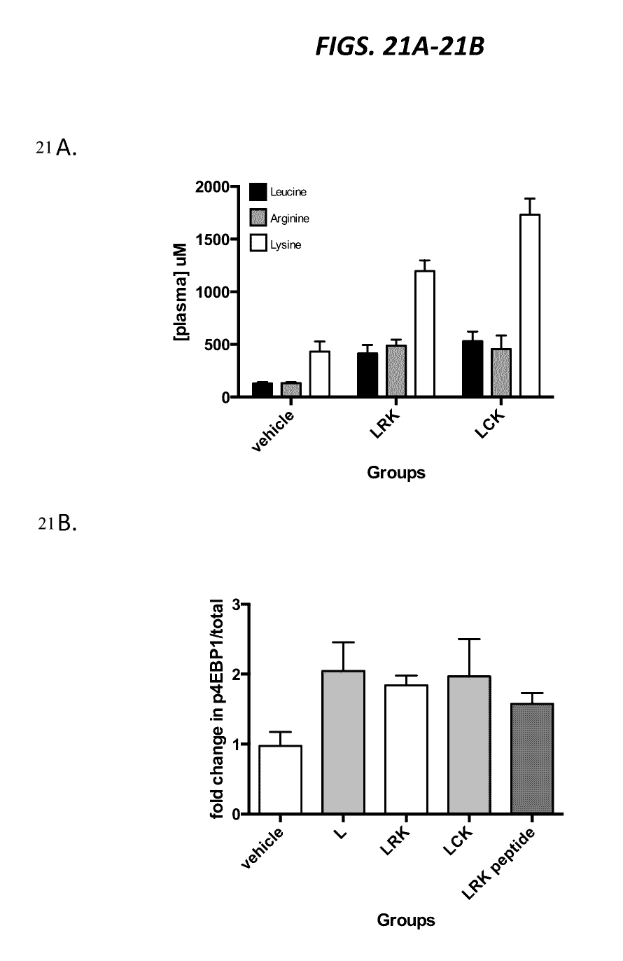

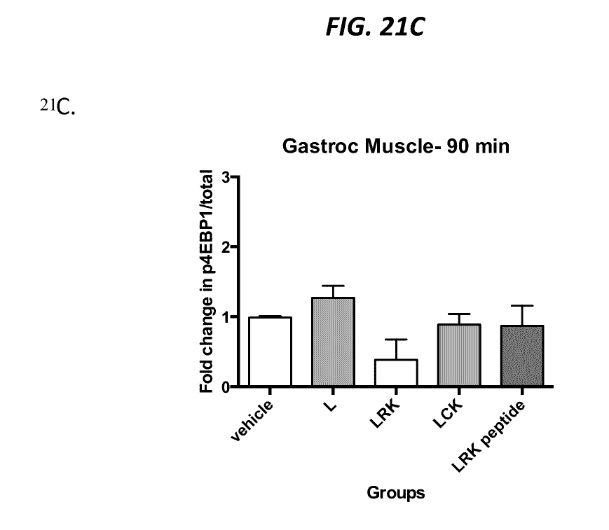

Mice were fasted overnight (18 hours) and sacrificed following a 30 min feeding (“Refed”) or injected with various combinations of amino acids or vehicle. Gastrocnemius muscles were collected and subjected to western blotting with antibodies for the indicated proteins in FIG. 18A. Free leucine levels were measured in the gastrocnemius muscle by LC-MS following indicated treatments. The results are shown in FIG. 18C. The LRK and LeeRK formulations were most potent in activating mTORC1 in the gastrocnemius as evident from increased phosphorylation of mTORC1 substrates. The citrulline containing formulations, LCitK and LeeCK only moderately activated mTORC1, but gastrocnemius amino acid measurements indicate muscle leucine and arginine levels were significantly lower compared to LRK and LeeRK. It seems that either the absence of arginine or the presence of citrulline interfered with uptake of leucine upon IP injection and there was little to no conversion of citrulline to arginine as expected (FIG. 18C). Given that LCitK formulations potently activated mTORC1 in multiple rat studies when dosed orally or intravenously (FIGS. 21, 23, 24 and data not shown) the inventors believe this lack of activation by LCitK and LeeCitK administered via IP injection in mice is due to a defect in amino acid absorption/uptake and would not be relevant to the efficacy of LCitK when dosed orally or intravenously. Similarly, LeeRK was slightly more potent than LRK as it resulted in increased levels of leucine in the gastrocnemius muscle (FIG. 18C).

In a separate experiment, mice were fasted overnight (18 hours) and then intravenously administered either a combination of leucine, arginine and lysine, or a combination of leucine ethyl ester, arginine and lysine, and then sacrificed at 15, 30, 60 or 120 minutes post injection. Fasted animals were injected with vehicle control and then sacrificed after 15 or 30 mins. Gastrocnemius muscles were collected from all mice and subjected to western blotting with the antibodies indicated in FIG. 18B. Free leucine levels were measured in the gastrocnemius muscle by LC-MS following indicated treatments. These results are shown in FIG. 18D.

Western blotting results demonstrate that while both LRK and LeeRK significantly activate mTORC1 in the gastrocnemius muscle, the duration of mTORC1 activation by LeeRK reaches 2 hours compared to 1.5 hours by LRK. This result is correlated to the increased concentration of intramuscular leucine upon administration of LeeRK.

### Example 7

### Effect of Oral Administration of Various Amino Acids or a Leu-Arg-Lys Tripeptide on Serum Amino Acid Levels and Muscle mTORC1 Activation in Fasted Rats

Sprague-Dawley rats have been established as a physiologically relevant pre-clinical model for measuring muscle protein synthetic rates in response to meals and protein supplements. Similar to humans, their anabolic muscle response is entirely dependent upon dietary protein intake (Yoshizawa F et al. Am J Physiol. 275, E814-20 (1998)); and furthermore, rats also experience an age-related decline in muscle mass and function (Kelleher A R et al. Am J. Physiol Endocrinol Metab. 304, E229-36 (2013)). In aged rats, like in elderly humans, muscles exhibit anabolic resistance, which is defined as a reduced muscle protein synthetic response to protein intake. Rats have also been invaluable models for the induction of anabolic resistance through limb immobilization, again mimicking what is observed in humans subjected to similar procedures.

To test for activation of mTORC1 signaling by oral administration of amino acids, rats exceeding 250 g were first fasted for 24 hours. This fasting period has been associated with inhibition of mTORC1 and a 40% decrease in muscle protein synthetic rates (Crozier S J et al. J Nutr. 135, 376-82 (2005)). After the fasting period, amino acid mixtures are administered via oral gavage at a volume of 10 ml/kg and rats were then sacrificed 30 and 90 minutes post dose for tissue and plasma collection. Previous publications have shown that oral ingestion of free essential amino acids or even leucine alone leads to an increase in mTORC1 activity and muscle protein synthesis within this period of time in a dose dependent manner (Crozier S J et al. J Nutr. 135, 376-82 (2005)).

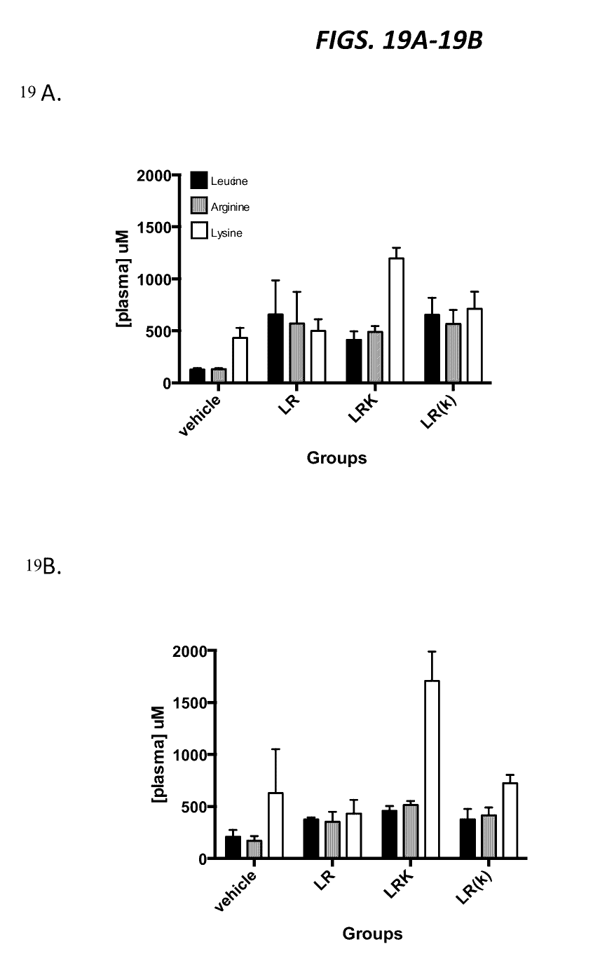

Given that equal molar amounts of L, R and K resulted in robust mTORC1 activation at the cellular level, our goal in these experiments was to orally dose the amino acids such that the plasma molar concentration of L, R and K were equal molar with the assumption that it would correlate with highest mTORC1 activity. Fasted plasma molar levels of lysine are higher than leucine and arginine, and the bioavailability of orally ingested lysine is superior to leucine and arginine (Fry C S et al. Curr Aging Sci. 4, 260-8 (2011)). As a result, we hypothesize that dosing lysine at a lower molar ratio to leucine and arginine will result in an equal plasma ratio of the 3 amino acids. We therefore compared three different formulations to leucine alone, one containing just leucine and arginine (LR), one with an equal molar amount of all three amino acids (LRK), and one ratio where we tested our hypothesis by adding ⅓ of a molar equivalent of lysine (LR(k)). All the amino acid mixtures contained equivalent amounts of leucine and rats (n=3 for each time point) were sacrificed 30 and 90 minutes after dosing. Amino acid levels in the plasma were measured by mass spectrometry and as seen in FIGS. 19A (30 minutes) and 19B (60 minutes), dosing lysine at a lower ratio to leucine and arginine resulted in a more equal molar ratio of all three amino acid compared to dosing equal molar amounts of all three amino acids.

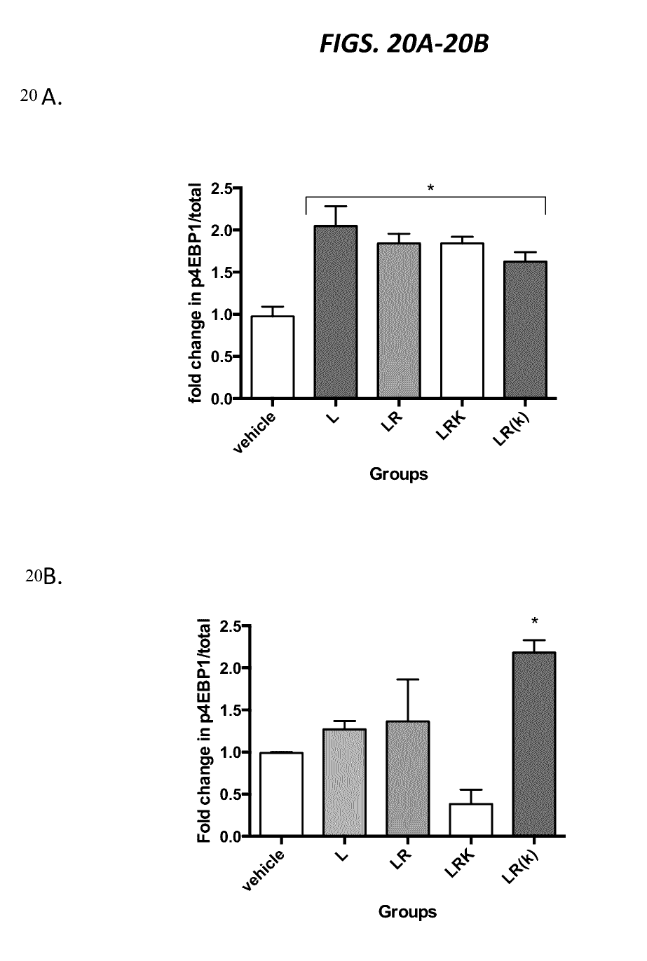

Gastrocnemius muscle was then collected from these rats and analyzed by SDS-PAGE followed by western blotting for levels of phosphorylated 4EBP1—an mTORC1 substrate. FIG. 20A demonstrates that at 30 minutes all formulations increase mTORC1 similarly. FIG. 20B shows that, after 90 minutes, dosing lysine at a lower ratio to leucine and arginine (LR(k)) resulted in the highest activation of mTORC1.

Another challenge in oral dosing of free amino acids is splanchnic extraction by primarily the gut and liver. Arginine is known to have a high rate of splanchnic extraction. Citrulline, an arginine precursor, is readily bioavailable and over 80% of circulating citrulline is converted to arginine by the kidneys (Bahri S. et al. Nutrition. 29, 479-84 (2013)). As a result, we hypothesized that oral dosing of citrulline (C) may substitute for arginine while bypassing mTORC1 activation in the liver. We compared an equal molar dosing of LRK to LCK and measured plasma amino acid levels and activation of mTORC1 in muscle. As shown in FIGS. 21A, 21B and 21C, LCK leads to comparable plasma levels of arginine as well as mTORC1 activation in muscle compared to LRK at both 30 (FIG. 21B) and 90 minutes (FIG. 21C). In addition, the activation of mTORC1 is approximately equal between LRK and LCK doses at 30 minutes, whereas LCK maintains greater activation of mTORC1 than LRK after 90 minutes.

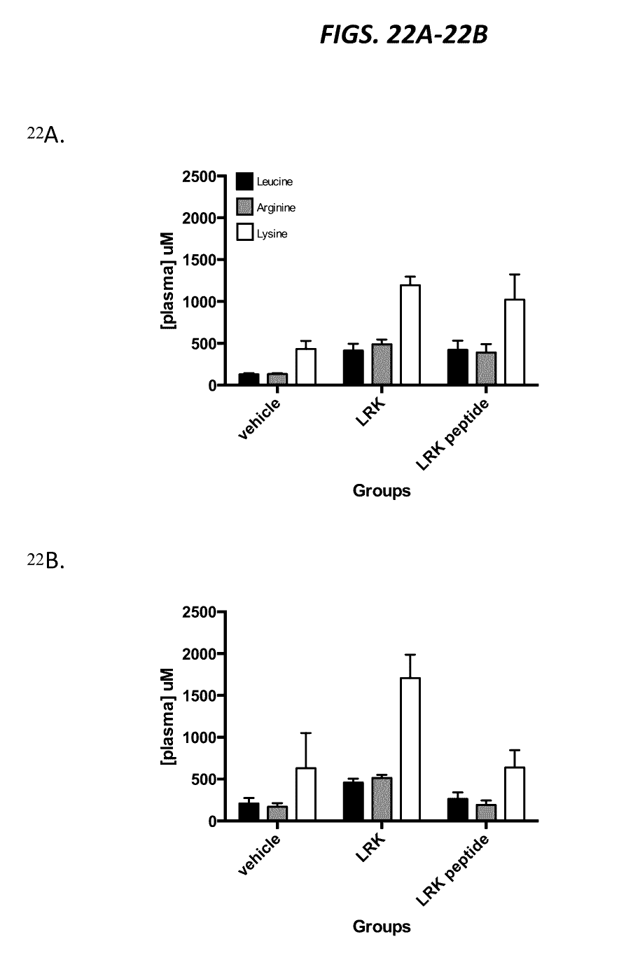

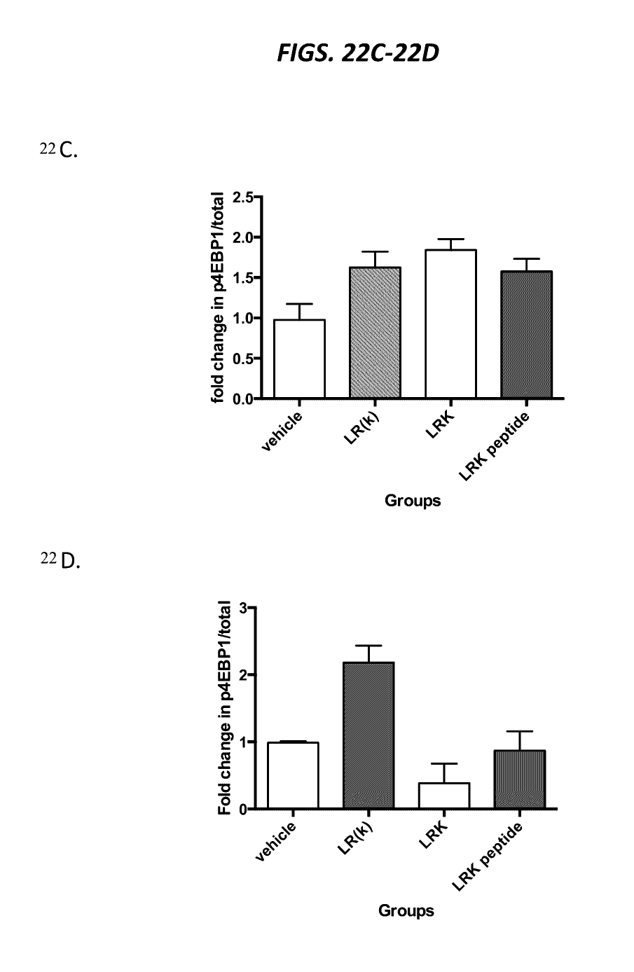

A peptide formulation of LRK was also tested for effect on serum amino acid levels and activation of mTORC1. During dietary intake, protein is broken down into di- and tri-peptide fragments and absorbed through intestinal peptide transporters. Peptides are highly soluble compared to free amino acids and have been demonstrated to have high bioavailability. A LRK tripeptide was synthesized at over 95% purity and dosed orally at a leucine-equivalent dose to the free amino acid mixture of LRK. As shown in FIGS. 22A, 22B, 22C and 22D, the LRK peptide led to similar plasma bioavailability and activation of mTORC1 compared to free amino acid LRK, but is less potent than the mixture of LRK where lysine is dosed at a lower molar ratio than leucine and arginine (“LR(k)”).

### Example 8

### Effect of Intravenous Administration of Various Amino Acids on Serum Amino Acid Levels, Muscle mTORC1 Activation in Fasted Rats

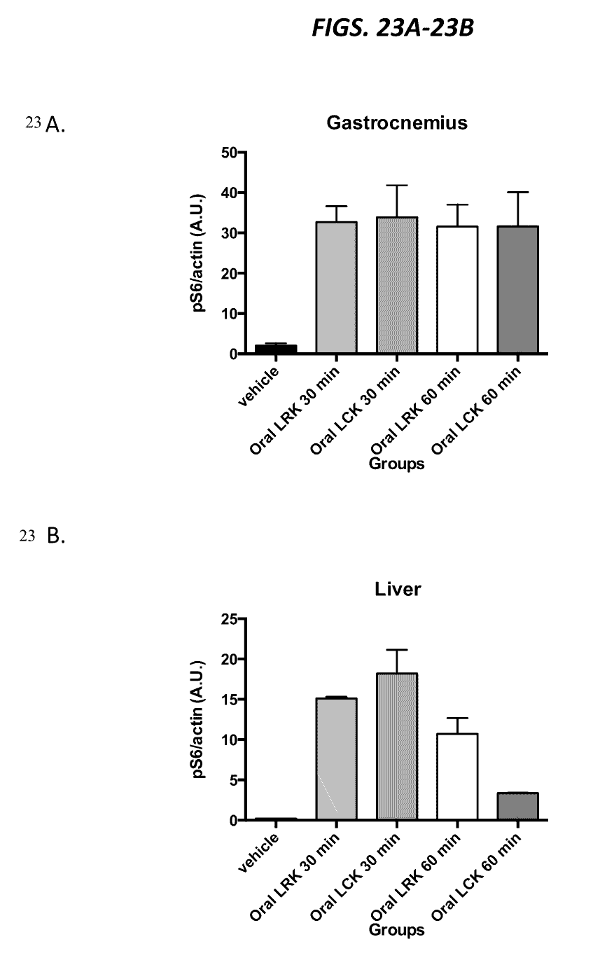

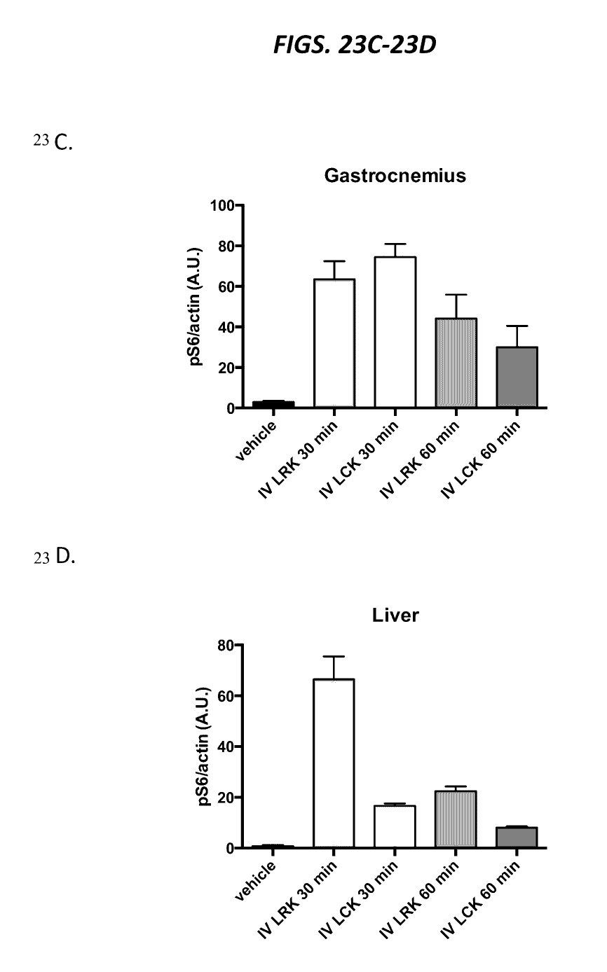

In addition to oral delivery of amino acids, intravenous (IV) delivery of LRK and LCK was tested for activation of mTORC1 in muscle and liver where lysine was dosed at one-third the molar amount of leucine and arginine. Rats (n=3), fasted for 24 hours, were administered LRK or LCK via oral gavage or bolus IV injection and gastrocnemius muscle and liver was isolated 30 and 60 minutes later. Gastrocnemius muscle and liver was analyzed by SDS-PAGE followed by western blotting for levels of phosphorylated S6—a marker of mTORC1 activity. As shown in FIGS. 23A and B, oral delivery of LRK or LCK resulted in equal activation of mTORC1 in the gastrocnemius muscle, but LCK administration resulted in a more transient activation in the liver. When the amino acid mixtures were administered IV, FIGS. 23C and D again show that both LRK and LCK significantly activate mTORC1 in gastrocnemius muscle. In the liver however, IV injection of LRK activated mTORC1 for a shorter duration compared to muscle while LCK showed little to no activation of mTORC1 in the liver (FIG. 23D).

### Example 9

### Effect of Administration of Various Amino Acids on Muscle Protein Synthesis in Fasted Rats

In summary, the above results demonstrate that addition of an equal molar amount of arginine and a third lower molar amount of lysine significantly augments the activation of mTORC1 signaling in skeletal muscle compared to leucine alone. Moreover, citrulline is an adequate substitute for arginine for mTORC1 activation at the level of muscle, but is less potent at activating mTORC1 in liver. However, it is unknown whether the increase in mTORC1 activation by LR(k) also leads to an increased induction of muscle protein synthesis.

To address this question, starved rats (n=8) were given oral doses of leucine, LR, LR(k), LC(k) and the LRK peptide and muscle protein synthetic rates were measured by administering a flooding dose of radiolabeled phenylalanine 10 minutes before sacrifice and quantifying uptake of radiolabeled phenylalanine.

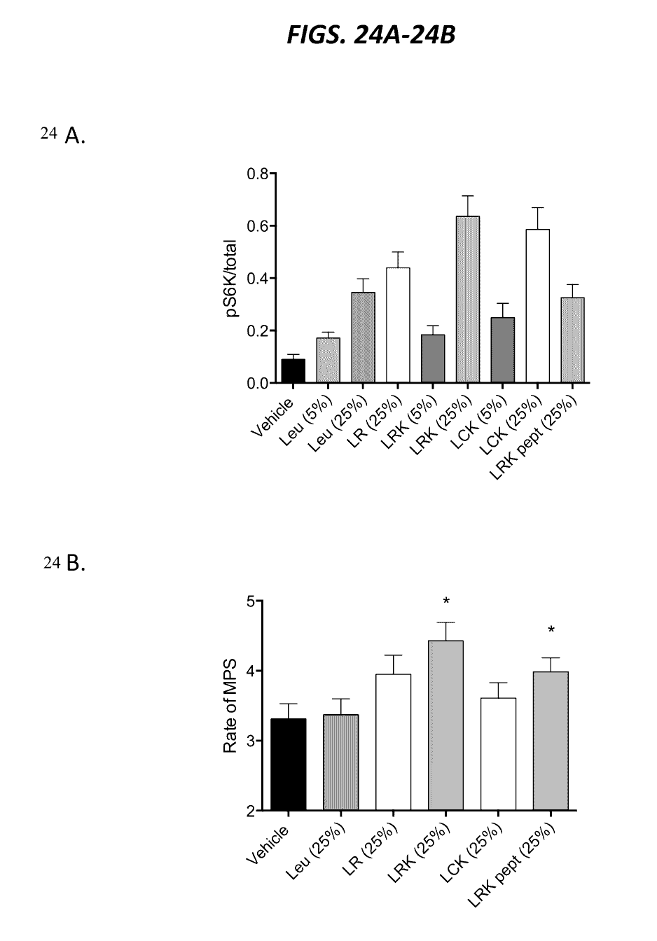

Starved rats were dosed with amino acids benchmarked to either 5% of 25% of their daily intake of leucine. Rats were sacrificed 45 minutes after oral gavage and tissues and plasma was collected for mTORC1 signaling assessment, quantification of plasma leucine levels and measurement of protein synthetic rates. As shown in FIG. 24A, LR(k) and LC(k), when dosed at the 25% concentration, were superior to leucine alone in activating mTORC1 in skeletal muscle.

Measuring protein synthetic rates in rats dosed with the 25% dose revealed that the increased mTORC1 activation by LR(k) and the LRK peptide significantly increased muscle protein synthesis (MPS) as shown in FIG. 24B. Surprisingly, fasted rats seemed to have elevated rates of MPS compared to historical data; as a result, there was little difference between fasted rats and rats given leucine alone in stark contrast to previously published results (Crozier, et al.). Administration of LC(k) increased muscle protein synthesis to a slightly greater degree than leucine alone, but the average rate did not approach significance due to the elevated baseline in the fasted vehicle control. This data indicates that LR(k) dosed as free amino acids or as a peptide are superior to leucine alone in activating mTORC1 and protein synthesis in muscle.

### REFERENCES

1. Zoncu, R., Efeyan, A. & Sabatini, D. M. mTOR: from growth signal integration to cancer, diabetes and ageing. Nature reviews. Molecular cell biology 12, 21-35 (2011).
2. Sengupta, S., Peterson, T. R. & Sabatini, D. M. Regulation of the mTOR complex 1 pathway by nutrients, growth factors, and stress. Molecular Cell 40, 310-322 (2010).
3. Sancak, Y., Peterson, T. R., Shaul, Y. D., Lindquist, R. A., Thoreen, C. C., Bar-Peled, L. & Sabatini, D. M. The Rag GTPases Bind Raptor and Mediate Amino Acid Signaling to mTORC1. Science 320, 1496-1501 (2008).
4. Sancak, Y., Bar-Peled, L., Zoncu, R., Markhard, A. L., Nada, S. & Sabatini, D. M. Ragulator-Rag Complex Targets mTORC1 to the Lysosomal Surface and Is Necessary for Its Activation by Amino Acids. Cell 141, 290-303 (2010).
5. Zoncu, R., Bar-Peled, L., Efeyan, A., Wang, S., Sancak, Y. & Sabatini, D. M. mTORC1 Senses Lysosomal Amino Acids Through an Inside-Out Mechanism That Requires the Vacuolar H⁺-ATPase. Science 334, 678-683 (2011).
6. Bar-Peled L., Schweitzer L. D., Zoncu R., & Sabatini D. M. Ragulator is a GEF for the rag GTPases that signal amino acid levels to mTORC1. Cell 150, 1196-208 (2012).
7. Bar-Peled L., Chantranupong L., et al. Science (2013).
8. Hara K., Yonezawa K., Weng Q.-P., Kozlowski M. T., Belham C., & Avruch J. Amino Acid Sufficiency and mTOR Regulate p70 S6 Kinase and eIF-4E BP1 through a Common Effector Mechanism. The Journal of Biological Chemistry 273, 14484-14494 (1998).
9. http://www.invitrogen.com/site/us/en/home/support/Product-Technical-Resources/media_formulation.119.html.
10. Zoncu, R., Efeyan, A. & Sabatini, D. M. mTOR: from growth signal integration to cancer, diabetes and ageing. Nat Rev Mol Cell Biol 12, 21-35 (2010).
11. Kim, E., Goraksha-Hicks, P., Li, L., Neufeld, T. P. & Guan, K. L. Regulation of TORC1 by Rag GTPases in nutrient response. Nat Cell Biol 10, 935-945 (2008).
12. Sancak, Y. et al. The Rag GTPases bind raptor and mediate amino acid signaling to mTORC1. Science 320, 1496-1501 (2008).
13. Sancak, Y. et al. Ragulator-Rag complex targets mTORC1 to the lysosomal surface and is necessary for its activation by amino acids. Cell 141, 290-303 (2010).
14. Zoncu, R. et al. mTORC1 senses lysosomal amino acids through an inside-out mechanism that requires the vacuolar H-ATPase. Science 334, 678-683 (2011).
15. Hirose, E., Nakashima, N., Sekiguchi, T. & Nishimoto, T. RagA is a functional homologue of S. cerevisiae Gtr1p involved in the Ran/Gsp1-GTPase pathway. J Cell Sci 111 (Pt 1), 11-21 (1998).
16. Kwiatkowski, D. J. et al. A mouse model of TSC1 reveals sex-dependent lethality from liver hemangiomas, and up-regulation of p70S6 kinase activity in Tsc1 null cells. Hum Mol Genet 11, 525-534 (2002).
17. Zhang, H. et al. Loss of Tsc1/Tsc2 activates mTOR and disrupts PI3K-Akt signaling through downregulation of PDGFR. J Clin Invest 112, 1223-1233 (2003).
18. Scheuner, D. et al. Translational control is required for the unfolded protein response and in vivo glucose homeostasis. Mol Cell 7, 1165-1176 (2001).
19. Birsoy, K. et al. Analysis of gene networks in white adipose tissue development reveals a role for ETS2 in adipogenesis. Development 138, 4709-4719 (2011).
20. Kuma, A. et al. The role of autophagy during the early neonatal starvation period. Nature 432, 1032-1036 (2004).
21. Komatsu, M. et al. Impairment of starvation-induced and constitutive autophagy in Atg7-deficient mice. J Cell Biol 169, 425-434 (2005).
22. Mizushima, N., Levine, B., Cuervo, A. M. & Klionsky, D. J. Autophagy fights disease through cellular self-digestion. Nature 451, 1069-1075 (2008).
23. Mortimore, G. E. & Schworer, C. M. Induction of autophagy by amino-acid deprivation in perfused rat liver. Nature 270, 174-176 (1977).
24. Kroemer, G., Marino, G. & Levine, B. Autophagy and the integrated stress response. Mol Cell 40, 280-293 (2010).
25. Roczniak-Ferguson, A. et al. The Transcription Factor TFEB Links mTORC1 Signaling to Transcriptional Control of Lysosome Homeostasis. Science signaling 5, ra42 (2012).
26. Settembre, C. et al. A lysosome-to-nucleus signalling mechanism senses and regulates the lysosome via mTOR and TFEB. EMBO J, doi:emboj201232 [pii]
27. 10.1038/emboj.2012.32 (2012).
28. Kalender, A. et al. Metformin, independent of AMPK, inhibits mTORC1 in a rag GTPase-dependent manner. Cell Metab 11, 390-401 (2010).
29. Choo, A. Y. et al. Glucose addiction of TSC null cells is caused by failed mTORC1-dependent balancing of metabolic demand with supply. Mol Cell 38, 487-499 (2010).
30. Shaw, R. J. et al. The tumor suppressor LKB1 kinase directly activates AMP-activated kinase and regulates apoptosis in response to energy stress. Proc Natl Acad Sci USA 101, 3329-3335 (2004).
31. Proud, C. G. eIF2 and the control of cell physiology. Semin Cell Dev Biol 16, 3-12 (2005).
32. Guerra, C. et al. Tumor induction by an endogenous K-ras oncogene is highly dependent on cellular context. Cancer Cell 4, 111-120 (2003).
33. Lo, S., Russell, J. C. & Taylor, A. W. Determination of glycogen in small tissue samples. J Appl Physiol 28, 234-236 (1970).
34. Yoshizawa F. et al. Effect of dietary protein on translation initiation in rat skeletal muscle and liver. Am J Physiol. 275, E814-20 (1998).
35. Kelleher A R. et al. The mTORC1 signaling repressors REDD½ are rapidly induced and activation of p70S6K1 by leucine is defective in skeletal muscle of an immobilized rat hindlimb. Am J Physiol Endocrinol Metab. 304, E229-36 (2013).
36. Crozier S J. et al. Oral leucine administration stimulates protein synthesis in rat skeletal muscle. J Nutr. 135, 376-82 (2005).
37. Fry C S, Rasmussen B B. Skeletal muscle protein balance and metabolism in the elderly. Curt Aging Sci. 4, 260-8 (2011).
38. Bahri S. et al. Citrulline: from metabolism to therapeutic use. Nutrition. 29, 479-84 (2013).
39. Wahl, S E et al, Mammalian Target of Rapamycin Promotes Oligodendrocyte Differentiation, Initiation and Extent of CNS Myelination. J Neurosci 34, 4453-65 (2014).
40. Norrmén C et al., mTORC1 Controls PNS Myelination along the mTORC1-RXRγ-SREBP-Lipid Biosynthesis Axis in Schwann Cells, Cell Rep. (2014) pii: S2211-1247(14)00769-4. doi: 10.1016/j.celrep.2014.09.001.
41. Pouwels, et al., “Hypomyelinating Leukodystrophies: Translational Research Progress and Propsects, Ann Neurol; 76:5-19 (2014).

What is claimed is:

1. A combination for increasing mTORC1 activity in a subject to increase muscle mass, increase muscle anabolism, or to treat a disease or condition selected from skeletal muscle atrophy, decreased satiety, abnormally high food intake, hyperphagia, ribosomopathies, cohesinopathies, and conditions that cause reduced myelination of nerves, the combination comprising:
a. a first component selected from L-arginine; an mTORC1 agonizing arginine mimetic; and a peptide, non-standard peptide, polypeptide, non-standard polypeptide, protein or non-standard protein any of which is enriched for one or both of L-arginine residues or mTORC1 agonizing arginine mimetic residues;
b. a second component selected from L-leucine; an mTORC1 agonizing leucine mimetic; and a peptide, non-standard peptide, polypeptide, non-standard polypeptide, protein or non-standard protein any of which is enriched for one or both of L-leucine residues or mTORC1 agonizing leucine mimetic residues; and
c. optionally, a third component selected from L-lysine; an mTORC1 agonizing lysine mimetic; and a peptide, non-standard peptide, polypeptide, non-standard polypeptide, protein or non-standard protein any of which is enriched for one or both of L-lysine residues or mTORC1 agonizing lysine mimetic residues;
wherein:
each component is present in an acceptable form for administration to the subject;
any two or all three components may be part of a single composition or a single molecule; and
each component is co-administered with one another to the subject.

2. The combination of claim 1, wherein the third component is administered to the subject.

3. The combination of claim 1 or 2, wherein at least one of the first component, second component or optional third component is other than a naturally occurring L-form of an amino acid.

4. The combination of any one of claims 1 to 3, wherein the first component is L-arginine or an mTORC1 agonizing arginine mimetic selected from a carboxy terminal modified form of L-arginine and a side-chain modified form of L-arginine.

5. The combination of claim 4, wherein the first component is selected from: L-arginine, a L-arginine ester, citrulline,

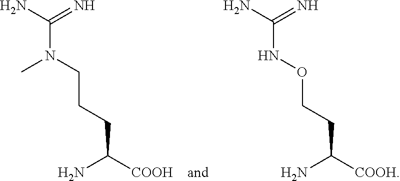

6. The combination of claim 5, wherein the L-arginine ester is L-arginine ethyl ester.

7. The combination of any one of claims 1-6, wherein the second component is L-leucine or an mTORC1 agonizing leucine mimetic selected from a carboxy terminal modified form of L-leucine, an amino terminal modified form of L-leucine, a side-chain modified form of L-leucine, and L-methionine.

8. The combination of claim 7, wherein the second component is selected from L-leucine, a L-leucine ester, L-methionine

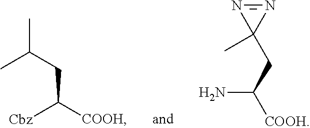

9. The combination of claim 8 wherein the L-leucine ester is L-leucine ethyl ester.

10. The combination of any one of claims 1-9, wherein the third component, if present, is selected from L-lysine and an L-lysine ester.

11. The combination of claim 10, wherein the L-lysine ester is L-lysine ethyl ester.

12. The combination of any one of claims 1-10, wherein at least one component is selected from:
a. a peptide, non-standard peptide, polypeptide, non-standard polypeptide, protein or non-standard protein any of which is enriched for one or both of L-arginine residues or mTORC1 agonizing arginine mimetic residues;
b. a peptide, non-standard peptide, polypeptide, non-standard polypeptide, protein or non-standard protein any of which is enriched for one or both of L-leucine residues or mTORC1 agonizing leucine mimetic residues; and
c. a peptide, non-standard peptide, polypeptide, non-standard polypeptide, protein or non-standard protein any of which is enriched for one or both of L-lysine residues or mTORC1 agonizing lysine mimetic residues; wherein: any peptide, non-standard peptide, polypeptide or non-standard polypeptide is optionally associated with one or more of a half-life increasing moiety, a cell penetration moiety, a specific organ-directing moiety, and a specific cell-type-directing moiety.

13. The combination of claim 12, wherein the at least one component is selected from:
a. a peptide, polypeptide, or protein any of which is enriched for L-arginine residues;
b. a peptide, polypeptide, or protein any of which is enriched for L-leucine residues; and
c. a peptide polypeptide, or protein any of which is enriched for L-lysine residues.

14. The combination of claim 13, wherein at least two components are independently selected from:
a. a peptide, non-standard peptide, polypeptide, non-standard polypeptide, protein or non-standard protein any of which is enriched for one or both of L-arginine residues or mTORC1 agonizing arginine mimetic residues;
b. a peptide, non-standard peptide, polypeptide, non-standard polypeptide, protein or non-standard protein any of which is enriched for one or both of L-leucine residues or mTORC1 agonizing leucine mimetic residues; and
c. a peptide, non-standard peptide, polypeptide, non-standard polypeptide, protein or non-standard protein any of which is enriched for one or both of L-lysine residues or mTORC1 agonizing lysine mimetic residues.

15. The combination of claim 14, wherein each of the at least two component is independently selected from:
a. a peptide, polypeptide, or protein any of which is enriched for L-arginine residues;
b. a peptide, polypeptide, or protein any of which is enriched for L-leucine residues; and
c. a peptide polypeptide, or protein any of which is enriched for L-lysine residues.

16. The combination of claim 12, wherein the third component is present and each of the first, second and third components are independently selected from:
a. a peptide, non-standard peptide, polypeptide, non-standard polypeptide, protein or non-standard protein any of which is enriched for one or both of L-arginine residues or mTORC1 agonizing arginine mimetic residues;
b. a peptide, non-standard peptide, polypeptide, non-standard polypeptide, protein or non-standard protein any of which is enriched for one or both of L-leucine residues or mTORC1 agonizing leucine mimetic residues; and
c. a peptide, non-standard peptide, polypeptide, non-standard polypeptide, protein or non-standard protein any of which is enriched for one or both of L-lysine residues or mTORC1 agonizing lysine mimetic residues.

17. The combination of claim 16, wherein each of the first, second and third components are independently selected from:
a. a peptide, polypeptide, or protein any of which is enriched for L-arginine residues;
b. a peptide, polypeptide, or protein any of which is enriched for L-leucine residues; and
c. a peptide polypeptide, or protein any of which is enriched for L-lysine residues.

18. The combination of claim 14 or 15, wherein the at least two components are present on one peptide, non-standard peptide, polypeptide, non-standard polypeptide, protein or non-standard protein which is enriched for at least two of:
a. L-arginine residues or mTORC1 agonizing arginine mimetic residues;
b. L-leucine residues or mTORC1 agonizing leucine mimetic residues; and
c. L-lysine residues or mTORC1 agonizing lysine mimetic residues.

19. The combination of claim 18, wherein the at least two components are present on one peptide, polypeptide, or protein which is enriched for at least two of:
a. L-arginine residues;
b. L-leucine residues; and
c. L-lysine residues.

20. The combination of claim 16 or 17, wherein each of the first, second and third components is present on one peptide, non-standard peptide, polypeptide, non-standard polypeptide, protein or non-standard protein which is enriched for
a. L-arginine residues or mTORC1 agonizing arginine mimetic residues;
b. L-leucine residues or mTORC1 agonizing leucine mimetic residues; and
c. L-lysine residues or mTORC1 agonizing lysine mimetic residues.

21. The combination of claim 20, wherein each of the first, second and third components is present on one peptide, polypeptide, or protein which is enriched for L-arginine residues, L-leucine residues, and L-lysine residues.

22. The combination of claim 21, wherein the one peptide is a tripepetide consisting of the amino acid sequence Leu-Arg-Lys.

23. The combination of any one of claims 18-22, wherein every component is present on the same peptide, non-standard peptide, polypeptide or non-standard polypeptide, and wherein
a. the peptide, non-standard peptide, polypeptide or non-standard polypeptide consists of residues selected from L-arginine residues, mTORC1 agonizing arginine mimetic residues, L-leucine residues or mTORC1 agonizing leucine mimetic residues, L-lysine residues and mTORC1 agonizing lysine mimetic residues;
b. the peptide, non-standard peptide, polypeptide or non-standard polypeptide is optionally associated with one or more of a half-life increasing moiety, a cell penetration moiety, a specific organ-directing moiety, and a specific cell-type-directing moiety.

24. The combination of any one of claims 12-23, wherein the peptide, non-standard peptide, polypeptide, non-standard polypeptide, protein or non-standard protein comprises a cell penetration amino acid sequence.

25. The combination of claim 24, wherein every component is present on one peptide or polypeptide, and wherein the peptide or polypeptide consists of residues selected from L-arginine residues, L-leucine residues and, L-lysine residues; and, optionally, is associated with one or more of a half-life increasing moiety, a cell penetration moiety, a specific organ-directing moiety, and a specific cell-type-directing moiety.

26. The combination of claim 23, wherein every component is present on one peptide or polypeptide, and wherein the peptide or polypeptide comprises at least one mTORC1 agonizing arginine mimetic residue, mTORC1 agonizing leucine mimetic residue, or mTORC1 agonizing lysine mimetic residue; and, optionally is associated with one of more of a half-life increasing moiety, a cell penetration moiety, a specific organ-directing moiety, and a specific cell-type-directing moiety.

27. The combination of claim 24, wherein the at least one mTORC1 agonizing arginine mimetic residue, mTORC1 agonizing leucine mimetic residue, or mTORC1 agonizing lysine mimetic residue is selected from a L-arginine ester, citrulline, 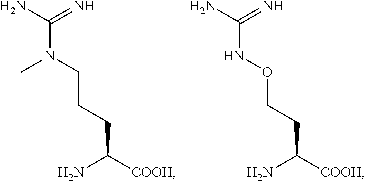
a L-leucine ester, L-methionine, 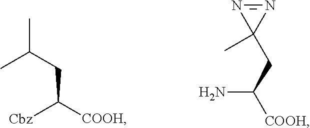
and corresponding monovalent and divalent radicals thereof.

28. The combination of any one of claims 23-27, wherein the peptide, non-standard peptide, polypeptide or non-standard polypeptide is between two and thirty residues in length.

29. The combination of claim 28, wherein the peptide, non-standard peptide, polypeptide or non-standard polypeptide is between two and twelve residues in length.

30. The combination of any one of claims 1-29, wherein each of the components is formulated into a pharmaceutically acceptable composition or a nutraceutical composition.

31. The combination of claim 30, wherein at least one of the components is formulated into a controlled release formulation.

32. The combination of claim 30 or 31, wherein at least one of the components is formulated into a composition to promote absorption from a specific portion of the digestive tract.

33. The combination of claim 30, wherein each component is formulated into either:
a. a controlled release formulation; or
b. a composition to promote absorption from a specific portion of the digestive tract.

34. The combination of claim 30, wherein at least one of the components is formulated into a pharmaceutical composition for delivery to a specific organ.

35. The combination of claim 34, wherein the specific organ is the brain and each pharmaceutical composition is formulated to either cross the blood-brain barrier or for direct administration to the CNS.

36. The combination of claim 24, wherein the specific organ is muscle.

37. The combination of claim 30, wherein each of the components is formulated into a composition for oral administration.

38. The combination of claim 30, wherein each of the components is formulated into a composition for parenteral or intra-muscular administration

39. The combination of claim 30, wherein each of the components is formulated into a composition that demonstrates an increase in Cₘₐₓ in the subject as compared to the Cₘₐₓ of a composition consisting of the corresponding component and a pharmaceutically acceptable buffer.

40. The combination of any one of claims 1-39, wherein prior to administration, information about the level of mTORC1 activity in the subject is received or obtained.

41. The combination of any one of claims 1-40, wherein prior to administration, information about whether the subject is deficient in lysine is received or obtained.

42. The combination of claim 41, wherein the serum or cellular lysine levels of the subject is obtained and the choice of administering the third component is determined based on the obtained lysine levels.

43. The combination of any one of claims 1-42, wherein each component is administered to the subject either a) prior to retiring for an extended sleep; or b) during sleeping hours.

44. The combination of any one of claims 1-42, wherein each component is administered to the subject only during waking hours.

45. The combination of any one of claims 1-42, wherein each component is administered to a fed subject.

46. The combination of any one of claims 1-42, wherein each component is administered to a fasted subject.

47. The combination of any one of claims 1-46, wherein the combination is used to promote muscle anabolism, improve muscle function, increase muscle mass, reverse muscle atrophy or to prevent muscle atrophy.

48. The combination of claim 47, wherein the combination is used to reverse muscle atrophy or to prevent muscle atrophy due to inactivity, immobilization, or age of the subject or a disease or condition suffered by the subject.

49. The combination of claim 48, wherein the combination is used to reverse muscle atrophy or to prevent muscle atrophy due to a broken bone, a severe burn, a spinal injury, an amputation, a degenerative disease, a condition wherein recovery requires bed rest for the subject, a stay in an intensive care unit, or long-term hospitalization.

50. The combination of claim 48, wherein the subject is suffering from a disease or condition known to be associated with cachexia and selected from cancer, AIDS, SARS, chronic heart failure, COPD, rheumatoid arthritis, liver disease, kidney disease and trauma.

51. The combination of claim 48, wherein the subject is suffering from a disease or condition known to be associated with malabsorption.

52. The combination of claim 51, wherein the disease or condition is selected from Crohn's disease, irritable bowel syndrome, celiac disease, and cystic fibrosis.

53. The combination of claim 48, wherein the subject is suffering from malnutrition, sarcopenia, muscle denervation, muscular dystrophy, an inflammatory myopathy, Spinal Muscle Atrophy, ALS, or myasthenia gravis.

54. The combination of claim 47, wherein the subject is preparing for, participating in or has recently returned from space travel.

55. The combination of claim 47, wherein the subject is preparing for, participating in or has recently returned from an armed conflict or military training.

56. The combination of any one of claims 1-46, wherein the combination is used to treat a ribosomopathy.

57. The combination of claim 56, wherein the ribosomopathy is selected from Diamond-Blackfan anemia, 5q-syndrome, Shwachman-Diamond syndrome, X-linked dyskeratosis, cartilage hair hypoplasia, and Treacher Collins syndrome.

58. The combination of any one of claims 1-46, wherein the combination is used to prevent autophagy in the patient.

59. The combination of claim 58, wherein the subject is suffering from cancer.

60. The combination of any one of claims 1-46, wherein the combination is used to induce satiety in the subject.

61. The combination of claim 60, wherein the subject is suffering from obesity, diabetes or metabolic syndrome.

62. The combination of claim 61, wherein the subject is suffering from obesity.

63. The combination of claim 47, wherein the combination is used to increase strength and/or to increase muscle mass following exercise.

64. The combination of claim 63, wherein the combination is carried out in conjunction with physical therapy, as part of total parenteral nutrition, or to promote functional electrical stimulation.

65. The combination of claim 63 or 64, wherein each of the components is present in a beverage or a nutrition bar.

66. The combination of any one of claims 1-65, wherein the subject is selected from a human and a companion animal.

67. The combination of claim 66, wherein the subject is selected from a human, a horse, a dog, or a cat.

68. The combination of claim 67, wherein the subject is a human.

69. The combination of any one of claims 1-46, wherein the combination is used to increase the muscle-to-fat ratio in a non-human animal.

70. The combination of any one of claims 1-46, wherein the combination is used to increase muscle mass in a non-human animal.

71. The combination of claim 69 or 70, wherein the non-human animal is selected from livestock or fish or poultry.

72. The combination of any one of claims 69-71, wherein each of the components is administered as an additive to the feed of the non-human animal.
